SEQUENCE LISTING (5 sequences)

SEQ ID NO: 1 - DNA, 30 bp, Artificial Sequence
  Description of Artificial Sequence Synthetic primer
gctagcgctagctcaatgtcctacacatgg

SEQ ID NO: 2 - DNA, 30 bp, Artificial Sequence
  Description of Artificial Sequence Synthetic primer
ctcgagctcgagcgtccatcggttggggag

SEQ ID NO: 3 - DNA, 51 bp, Artificial Sequence
  Description of Artificial Sequence Synthetic primer
gctagggctagccaccaccaccaccaccactcaatgtcctacacatggaca

SEQ ID NO: 4 - DNA, 36 bp, Artificial Sequence
  Description of Artificial Sequence Synthetic primer
ctcgagctcgagtcaacggggtcgggcacgagacag

SEQ ID NO: 5 - DNA, 15 bp, Artificial Sequence
  Description of Artificial Sequence Synthetic primer
ttttttttttttttt

(19) United States
(12) Patent Application Publication
(10) Pub. No.: US 2013/0136719 A1
(43) Pub. Date: May 30, 2013

(54) COMPOUNDS AND METHODS FOR THE TREATMENT OR PREVENTION OF FLAVIVIRUS INFECTIONS

(71) Applicants: Laval Chan Chun Kong, Kirkland (CA); Sanjoy Kumar Das, Pierrefonds (CA); Carl Poisson, Montreal (CA)

(72) Inventors: Laval Chan Chun Kong, Kirkland (CA); Sanjoy Kumar Das, Pierrefonds (CA); Carl Poisson, Montreal (CA)

(73) Assignee: VERTEX PHARMACEUTICALS INCORPORATED, Cambridge, MA (US)

(21) Appl. No.: 13/480,631

(22) Filed: May 25, 2012

Related U.S. Application Data
(63) Continuation of application No. PCT/US2010/057719, filed on Nov. 23, 2010.
(60) Provisional application No. 61/374,396, filed on Aug. 17, 2010.
(60) Provisional application No. 61/264,305, filed on Nov. 25, 2009.

(51) Int. Cl. C07D 333/40; C07D 409/12; A61K 31/4192; A61K 31/403; C07D 409/14; A61K 31/506; A61K 45/06; A61K 31/4196; A61K 31/381; A61K 31/4535

(52) U.S. Cl. CPC C07D 333/40; A61K 31/381; C07D 409/12; A61K 31/4192; A61K 31/4535; C07D 409/14; A61K 31/506; A61K 45/06; A61K 31/4196; A61K 31/403
USPC 424/85.7; 514/447; 549/69; 548/255; 514/359; 546/213; 514/326; 544/331; 514/275; 424/184.1; 514/44 A; 548/266.2; 514/383; 549/60; 514/444; 548/408; 514/409

(57) ABSTRACT

Compounds represented by formula I:

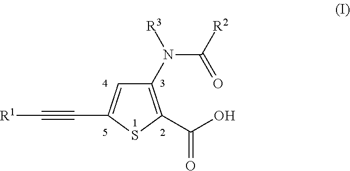

or pharmaceutically acceptable salts thereof, wherein R¹, R², and R³ are as defined herein, are useful for treating flaviviridae viral infections.

### RELATED APPLICATIONS

This application claims the benefit of PCT application number PCT/US2010/057719, filed Nov. 23, 2010, which claims priority to U.S. Provisional Application No. 61/264,305, filed on Nov. 25, 2009 and U.S. Provisional Application No. 61/374,396, filed on Aug. 17, 2010. The entire contents of these applications are incorporated herein by reference.

### BACKGROUND OF THE INVENTION

The present invention relates to novel compounds and a method for the treatment or prevention of Flavivirus infections using novel compounds.

Hepatitis is a disease occurring throughout the world. It is generally of viral nature, although there are other causes known. Viral hepatitis is by far the most common form of hepatitis. Nearly 750,000 Americans are affected by hepatitis each year, and out of those, more than 150,000 are infected with the hepatitis C virus (“HCV”).

HCV is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co- and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

The only treatment currently available for HCV infection is interferon-α (IFN-α). However, according to different clinical studies, only 70% of treated patients normalize alanine aminotransferase (ALT) levels in the serum and after discontinuation of IFN, 35% to 45% of these responders relapse. In general, only 20% to 25% of patients have long-term responses to IFN. Clinical studies have shown that combination treatment with IFN and ribavirin (RIBA) results in a superior clinical response to that of IFN alone.

There is therefore a great need for the development of anti-viral agents for use in treating or preventing Flavivirus infections.

### SUMMARY OF THE INVENTION

The present invention generally relates to compounds useful for treating or preventing Flavivirus infections, such as HCV infections.

In one embodiment, the present application is directed to a compound of formula (I) or a pharmaceutically acceptable salt thereof:

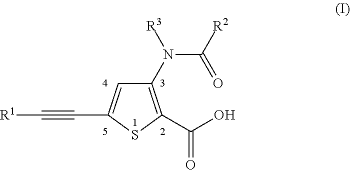

wherein,
R¹ is C₁₋₆ alkyl or C₃₋₆ cycloalkyl;
R² is a phenyl which is unsubstituted or substituted one or more times by R¹⁰;
R³ is C₁₋₆ alkyl which is unsubstituted or substituted one or more times by R¹¹; 6 membered heterocycle which is unsubstituted or substituted one or more times by R¹², C₃₋₆ cycloalkyl which is unsubstituted or substituted one or more times by R¹²; or

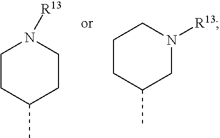

R¹⁰ is halogen, C₁₋₃ alkyl, halogenated C₁₋₃ alkyl, C₁₋₃-alkoxy, —NH₂, hydroxyl, nitro, cyano or CH₃COO—;
R¹¹ is halogen, oxo, C₁₋₆ alkoxy, C₃₋₆ cycloalkyl, 5-6 membered heterocycle, —NH₂, —NH(C₁₋₄ alkyl), —N(C₁₋₄ alkyl)₂, —CONH₂, —CONH(C₁₋₄ alkyl), —CON(C₁₋₄ alkyl)₂, alkyl)COC₁₋₄alkyl, —NHCOC₁₋₄ alkyl, carboxy, hydroxyl, nitro, azido, cyano, —S(O)₀₋₂H, —S(O)₀₋₂C₁₋₄ alkyl, —NHSO₂C₁₋₄ alkyl;
R¹² is OH, oxo, halogen, C₁₋₆-alkoxy, C₁₋₆-alkyl, C₁₋₆-alkyl-CO—NH—, C₁₋₆-alkyl-CO—N(C₁₋₆-alkyl)-, 3-6 membered heterocycle, or a 5-10 membered heteroaryl; and
R¹³ is C₁₋₆-alkyl, C₃₋₇-cycloalkyl, halogenated C₁₋₆-alkyl, C₁₋₆-alkyl-CO—, —S(O)₀₋₂C₁₋₄ alkyl, 5-10 membered heteroaryl or C₆₋₁₄-aryl.

In another embodiment, the invention is directed to a pharmaceutical composition comprising a compound of the invention described herein and a pharmaceutically acceptable carrier or excipient.

In yet another embodiment, the invention provides methods of treating a HCV infection in a subject, comprising administering to the subject a therapeutically effective amount of a compound of the invention described herein.

In yet another embodiment, the invention is directed to a method of inhibiting or reducing the activity of HCV polymerase in a subject, comprising administering to the subject a therapeutically effective amount of a compound of the invention described herein.

In yet another embodiment, the invention is directed to a method of inhibiting or reducing the activity of HCV polymerase in a biological in vitro sample, comprising administering to the sample an effective amount of a compound of the invention described herein.

The present invention also provides use of the compounds of the invention described herein for the manufacture of the medicament for treating a HCV infection in a subject, or for inhibiting or reducing the activity of HCV polymerase in a subject.

Also provided herein is use of the compounds of the invention described herein for treating a HCV infection in a subject, or for inhibiting or reducing the activity of HCV polymerase in a subject.

### DETAILED DESCRIPTION OF THE INVENTION

In one aspect, the present invention provides a compound of formula I:

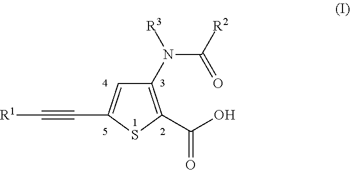

or a pharmaceutically acceptable salt thereof;
wherein,
R¹ is C₁₋₆ alkyl or C₃₋₆ cycloalkyl;
R² is a phenyl which is unsubstituted or substituted one or more times by R¹⁰;
R³ is C₁₋₆ alkyl which is unsubstituted or substituted one or more times by R¹¹; 6 membered heterocycle which is unsubstituted or substituted one or more times by R¹², C₃₋₆ cycloalkyl which is unsubstituted or substituted one or more times by R¹²; or

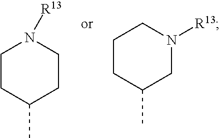

R¹⁰ is halogen, C₁₋₃ alkyl, halogenated C₁₋₃ alkyl, C₁₋₃-alkoxy, —NH₂, hydroxyl, nitro, cyano or CH₃COO—;
R¹¹ is halogen, oxo, C₁₋₆ alkoxy, C₃₋₆ cycloalkyl, 5-6 membered heterocycle, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄, alkyl, —NHCOC₁₋₄ alkyl, carboxy, hydroxyl, nitro, azido, cyano, —S(O)₀₋₂H, —S(O)₀₋₂C₁₋₄ alkyl, —NHSO₂C₁₋₄ alkyl;
R¹² is OH, oxo, halogen, C₁₋₆-alkoxy, C₁₋₆-alkyl, C₁₋₆-alkyl-CO—NH—, C₁₋₆-alkyl-CO—N(C₁₋₆-alkyl)-, 3-6 membered heterocycle, or a 5-10 membered heteroaryl.
R¹³ is C₁₋₆-alkyl, C₃₋₇-cycloalkyl, halogenated C₁₋₆-alkyl, C₁₋₆-alkyl-CO—, —S(O)₀₋₂C₁₋₄ alkyl, 5-10 membered heteroaryl or C₆₋₁₄-aryl.

In one embodiment, compounds of the present invention comprise those wherein the following embodiments are present, either independently or in combination.

According to a further embodiment, R¹ is C₁₋₆ alkyl.

According to a further embodiment, R¹ is C₃₋₆ cycloalkyl.

According to a further embodiment, R¹ is methyl, ethyl, propyl, isopropyl, butyl, sec-butyl, or tert-butyl.

According to a further embodiment, R¹ is tert-butyl.

According to a further embodiment, R¹ is cyclopropyl, cyclobutyl, cyclopentyl, or cyclohexyl.

According to a further embodiment, R² is phenyl which is disubstituted.

According to a further embodiment, R² is phenyl which is substituted in 2 and 4 position.

According to a further embodiment, R² is phenyl which is substituted in 4 position.

According to a further embodiment, R² is 2,4-dichlorophenyl, 2-fluoro-4-chlorophenyl, 2,4-dimethylphenyl, 2-hydroxy-4-methylphenyl, 2-methyl-4-chlorophenyl, 2-bromo-4-methylphenyl, 3-fluoro-4-methylphenyl, 2-amino-4-chlorophenyl, 4-chlorophenyl, 4-methylphenyl or 4-trifluoromethylphenyl.

According to a further embodiment, R² is 4-chlorophenyl, 4-methylphenyl or 4-trifluoromethylphenyl.

According to a further embodiment, R² is 2,4-dichlorophenyl, 2-methyl-4-chlorophenyl or 2-amino-4-chlorophenyl.

According to a further embodiment, R² is 2,4-dichlorophenyl.

According to a further embodiment, R² is phenyl.

According to a further embodiment, R³ is C₁₋₆ alkyl which is unsubstituted or substituted one or more times by R¹¹.

According to a further embodiment, R³ is 6 membered heterocycle which is unsubstituted or substituted one or more times by R¹².

According to a further embodiment, R³ is C₃₋₆ cycloalkyl which is unsubstituted or substituted one or more times by R¹²

According to a further embodiment, R³ is cyclohexyl which is unsubstituted or substituted one or more times by R¹².

According to a further embodiment, R³ is methyl, ethyl, propyl, isopropyl, cyclopropyl, methylcyclopropyl, cyclobutyl, butyl, sec-butyl, tert-butyl, pentyl or cyclopentyl.

According to a further embodiment, R³ is isopropyl.

According to a further embodiment, R³ is methyltetrahydropyranyl.

According to a further embodiment, R³ is methyltetrahydrofuranyl.

According to a further embodiment, R³ is 1,3-dimethoxy isopropyl, 1-methoxy isopropyl, methoxy ethyl, 2,2,2-trifluoroethyl, 2,2-difluoroethyl or 2-fluoroethyl.

According to a further embodiment, R³ is tetrahydrofuran, tetrahydropyran or 1,3-dioxane.

According to a further embodiment, R³ is cyclohexyl which is substituted in the 4-position by OH, oxo, halogen, C₁₋₆-alkoxy, C₁₋₆-alkyl, C₁₋₆-alkyl-CO—NH—, C₁₋₆-alkyl-CO—N(C₁₋₆-alkyl)-, 3-6 membered heterocycle, or a 5-10 membered heteroaryl, wherein the 4-position substituent is in the trans position relative to the amino group.

According to a further embodiment, R³ is cyclohexyl which is substituted in the 4-position by OH, C₁₋₆-alkyl, or C₁₋₆-alkoxy wherein the 4-position substituent is in the trans position relative to the amino group.

According to a further embodiment, R³ is cyclohexyl which is substituted in the 4-position by 1,2,4-triazolyl or 1,2,3-triazolyl, and the 4-position substituent is in the trans position relative to the amino group.

According to a further embodiment, R³ is cyclohexyl which is substituted one or more times by OH, oxo, halogen, C₁₋₆-alkoxy, C₁₋₆-alkyl, or triazolyl.

According to a further embodiment, R³ is cyclohexyl which is substituted one or more times by OH, halogen, C₁₋₆-alkyl, or C₁₋₆-alkoxy.

According to a further embodiment, R³ is cyclohexyl which is substituted one or more times by OH or C₁₋₆-alkoxy.

According to a further embodiment, R³ is cyclohexyl which is substituted one or more times by OH.

According to a further embodiment, R³ is cyclohexyl which is substituted one or more times by C₁₋₆-alkoxy.

According to a further embodiment, R³ is cyclohexyl which is substituted one or more times by methoxy, ethoxy, propyloxy, isopropyloxy, or butyloxy.

According to a further embodiment, R³ is cyclohexyl which is substituted by a triazolyl.

According to a further embodiment, R³ is cyclohexyl which is substituted in the 4-position by OH or C₁₋₆-alkoxy.

According to a further embodiment, R³ is cyclohexyl which is substituted in the 4-position.

According to a further embodiment, R³ is cyclohexyl which is substituted in the 4-position and the 4-position substituent is in the trans position relative to the amino group.

According to a further embodiment, R³ is cyclohexyl which is substituted in the 4-position and the 4-position substituent is in the cis position relative to the amino group.

According to a further embodiment, R³ is

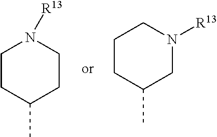

and R¹³ is C₁₋₆-alkyl, C₃₋₇-cycloalkyl, halogenated C₁₋₆-alkyl, C₁₋₆-alkyl-CO—, —S(O)₀₋₂C₁₋₄ alkyl, 5-10 membered heteroaryl or C₆₋₁₄-aryl.

According to a further embodiment, R³ is

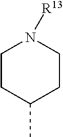

and R¹³ is C₁₋₆-alkyl, C₃₋₇-cycloalkyl, halogenated C₁₋₆-alkyl, C₁₋₆-alkyl-CO—, —S(O)₀₋₂C₁₋₄ alkyl, 5-10 membered heteroaryl or C₆₋₁₄-aryl.

According to a further embodiment, R³ is

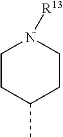

and R¹³ is methyl, ethyl, propyl or isopropyl.

According to a further embodiment, R¹⁰ is halogen, C₁₋₃ alkyl or —NH₂. According to a further embodiment, R¹⁰ is halogen.

According to a further embodiment, R¹⁰ is chloro, methyl or —NH₂.

According to a further embodiment, R¹⁰ is chloro.

According to a further embodiment, R¹¹ is halogen, oxo, C₁₋₆ alkoxy, C₃₋₆ cycloalkyl, 5-6 membered heterocycle, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄, alkyl, —NHCOC₁₋₄ alkyl or hydroxyl.

According to a further embodiment, R¹¹ is halogen, C₁₋₆ alkoxy, C₃₋₆ cycloalkyl, —NH₂, —NH(C₁₋₄ alkyl), —N(C₁₋₄ alkyl)₂, or hydroxyl.

According to a further embodiment, R¹¹ is 5-6 membered heterocycle.

According to a further embodiment, R¹¹ is halogen, methoxy or hydroxyl.

According to a further embodiment, R¹¹ is fluoro or methoxy.

According to a further embodiment, R¹¹ is fluoro.

According to a further embodiment, R¹¹ is methoxy.

According to a further embodiment, R¹² is OH, oxo, halogen, C₁₋₃-alkoxy, C₁₋₃-alkyl, C₁₋₃-alkyl-CO—NH—, C₁₋₃-alkyl-CO—N(C₁₋₃-alkyl)-, 3-6 membered heterocycle, or a 5-10 membered heteroaryl.

According to a further embodiment, R¹² is methyl, ethyl, propyl, isopropyl, cyclopropyl, cyclobutyl, butyl, sec-butyl, or tert-butyl.

According to a further embodiment, R¹² is methyl, ethyl, or isopropyl.

According to a further embodiment, R¹² is OH, methoxy or ethoxy.

According to a further embodiment, R¹² is OH.

According to a further embodiment, R¹² is methoxy.

According to a further embodiment, R¹² is halogen.

According to a further embodiment, R¹² is fluoro.

According to a further embodiment, R¹² is triazolyl.

According to a further embodiment, R¹² is 1,2,4-triazolyl or 1,2,3-triazolyl.

According to a further embodiment, R¹² is spiropyrrolidinone or N-methyl spiropyrrolidinone.

In accordance with a preferred embodiment of the invention, the compounds of the present invention are selected from the compounds of formula (I) and pharmaceutically acceptable salts thereof, wherein:
R¹ is C₁₋₆ alkyl;
R² is phenyl which is unsubstituted or substituted one or more times by halogen, C₁₋₃ alkyl or —NH₂; and
R³ is C₁₋₆ alkyl which is unsubstituted or substituted one or more times by halogen, oxo, C₁₋₆ alkoxy, C₃₋₆ cycloalkyl, 5-6 membered heterocycle, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄, alkyl, —NHCOC₁₋₄ alkyl, carboxy, hydroxyl, nitro, azido, cyano, —S(O)₀₋₂H, —S(O)₀₋₂C₁₋₄ alkyl, or —NHSO₂C₁₋₄ alkyl.

In accordance with a preferred embodiment of the invention, the compounds of the present invention are selected from the compounds of formula (I) and pharmaceutically acceptable salts thereof, wherein:
R¹ is C₁₋₆ alkyl;
R² is phenyl which is unsubstituted or substituted one or more times by halogen; and
R³ is C₁₋₆ alkyl which is unsubstituted or substituted one or more times by halogen, oxo, C₁₋₆ alkoxy, C₃₋₆ cycloalkyl, 5-6 membered heterocycle, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄, alkyl, —NHCOC₁₋₄ alkyl, carboxy, hydroxyl, nitro, azido, cyano, —S(O)₀₋₂H, —S(O)₀₋₂C₁₋₄ alkyl, or —NHSO₂C₁₋₄ alkyl.

In accordance with a preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl; and
R³ is C₁₋₆ alkyl which is unsubstituted or substituted one or more times by halogen, oxo, C₁₋₆ alkoxy, C₃₋₆ cycloalkyl, 5-6 membered heterocycle, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄, alkyl, —NHCOC₁₋₄ alkyl, carboxy, hydroxyl, nitro, azido, cyano, —S(O)₀₋₂H, —S(O)₀₋₂C₁₋₄ alkyl, or —NHSO₂C₁₋₄ alkyl.

In accordance with a preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl; and
R³ is C₁₋₆ alkyl which is unsubstituted or substituted one or more times by halogen, C₁₋₆ alkoxy, —NH₂, —NH(C₁₋₄ alkyl), or —N(C₁₋₄ alkyl)₂.

In accordance with a preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl; and
R³ is C₁₋₆ alkyl which is unsubstituted or substituted one or more times by halogen or C₁₋₆ alkoxy.

In accordance with a preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl; and
R³ is C₁₋₆ alkyl which is unsubstituted or substituted one or more times by fluoro or methoxy.

In accordance with a preferred embodiment of the invention, the compounds of the present invention are selected from the compounds of formula (I) and pharmaceutically acceptable salts thereof, wherein:
R¹ is C₁₋₆ alkyl;
R² is phenyl which is unsubstituted or substituted one or more times by halogen, C₁₋₃ alkyl or —NH₂; and
R³ is cyclohexyl which is substituted one or more times by OH, halogen, C₁₋₄-alkoxy, C₁₋₄-alkyl, C₁₋₄-alkyl-CO—NH—, C₁₋₄-alkyl-CO—N(C₁₋₄-alkyl)-, or triazolyl.

In accordance with a preferred embodiment of the invention, the compounds of the present invention are selected from the compounds of formula (I) and pharmaceutically acceptable salts thereof, wherein:
R¹ is C₁₋₆ alkyl;
R² is phenyl which is unsubstituted or substituted one or more times by halogen; and
R³ is cyclohexyl which is substituted one or more times by OH, halogen (e.g., F), C₁₋₄-alkoxy, C₁₋₄-alkyl, C₁₋₄-alkyl-CO—NH—, C₁₋₄-alkyl-CO—N(C₁₋₄-alkyl)-, or triazolyl.

In accordance with another preferred embodiment of the invention, the compounds of the present invention are selected from the compounds of formula (IB) and pharmaceutically acceptable salts thereof, wherein:
R¹ is C₁₋₆ alkyl;
R² is 2,4-dichlorophenyl; and
R³ is cyclohexyl which is substituted in the 4-position by OH, halogen, C₁₋₄-alkoxy, C₁₋₄-alkyl, C₁₋₄-alkyl-CO—NH—, C₁₋₄-alkyl-CO—N(C₁₋₄-alkyl)-, or triazolyl.

In accordance with another preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl; and
R³ is cyclohexyl which is substituted in the 4-position by OH, halogen, C₁₋₄-alkoxy, C₁₋₄-alkyl, C₁₋₄-alkyl-CO—NH—, C₁₋₄-alkyl-CO—N(C₁₋₄-alkyl)-, or triazolyl and the 4-position substituent is in the trans position relative to the amino group.

In accordance with another preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl; and
R³ is cyclohexyl which is substituted one or more times by OH, F, C₁₋₄-alkoxy, C₁₋₄-alkyl, or halogenated C₁₋₄-alkyl.

In accordance with another preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl; and
R³ is cyclohexyl which is substituted one or more times by OH, F, C₁₋₄-alkoxy, or C₁₋₄-alkyl.

In accordance with another preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl; and
R³ is cyclohexyl which is substituted one or more times by OH, F, methoxy or methyl.

In accordance with another preferred embodiment of the invention, the compounds of the present invention are selected from the compounds of formula (I) and pharmaceutically acceptable salts thereof, wherein:
R¹ is methyl, ethyl, propyl, isopropyl, butyl, sec-butyl or tert-butyl;
R² is 2,4-dichlorophenyl; and
R³ is cyclohexyl which is substituted one or more times by OH, F, C₁₋₄-alkoxy, or C₁₋₄-alkyl.

In accordance with another preferred embodiment of the invention, the compounds of the present invention are selected from the compounds of formula (I) and pharmaceutically acceptable salts thereof, wherein:
R¹ is tert-butyl;
R² is 2,4-dichlorophenyl; and
R³ is cyclohexyl which is substituted one or more times by OH, F, C₁₋₄-alkoxy, or C₁₋₄-alkyl.

In accordance with another preferred embodiment of the invention, the compounds of the present invention are selected from the compounds of formula (I) and pharmaceutically acceptable salts thereof, wherein:
R¹ is C₁₋₆ alkyl;
R² is phenyl which is unsubstituted or substituted one or more times by halogen, C₁₋₃ alkyl or —NH₂;
R³ is

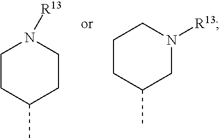

and
R¹³ is C₁₋₆-alkyl, C₃₋₇-cycloalkyl, halogenated C₁₋₆-alkyl, C₁₋₆-alkyl-CO—, —S(O)₀₋₂C₁₋₄ alkyl, heteroaryl or C₆₋₁₄-aryl.

In accordance with another preferred embodiment of the invention, the compounds of the present invention are selected from the compounds of formula (I) and pharmaceutically acceptable salts thereof, wherein:
R¹ is C₁₋₆ alkyl;
R² is phenyl which is unsubstituted or substituted one or more times by halogen;
R³ is

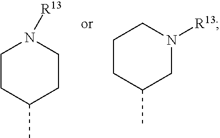

and
R¹³ is C₁₋₆-alkyl, C₃₋₇-cycloalkyl, halogenated C₁₋₆-alkyl, C₁₋₆-alkyl-CO—, —S(O)₀₋₂C₁₋₄ alkyl, heteroaryl or C₆₋₁₄-aryl.

In accordance with another preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl;
R³ is

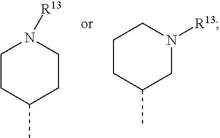

and
R¹³ is C₁₋₆-alkyl or halogenated C₁₋₆-alkyl.

In accordance with another preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl;
R³ is

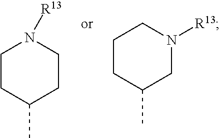

and
R¹³ is C₁₋₆-alkyl.

In accordance with another preferred embodiment of the invention, the compounds of the present invention are selected from the compounds of formula (I) and pharmaceutically acceptable salts thereof, wherein:
R¹ is C₁₋₆ alkyl;
R² is 2,4-dichlorophenyl;
R³ is

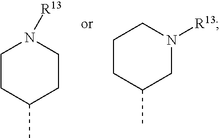

and
R¹³ is methyl, ethyl, propyl, isopropyl, butyl, sec.-butyl, or tert.-butyl.

According to an aspect of the invention, the compounds representative of the invention are selected from:

TABLE 1
List of compound representative of the invention

| Cpd | | |
|---|---|---|
| # | Structure | Name |
| 1 | 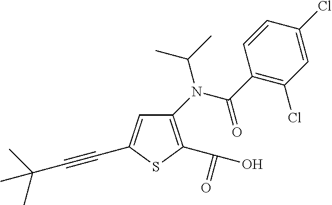 [SMILES: CC(C)N(C(=O)c1ccc(Cl)cc1Cl)c1cc(C#CC(C)(C)C)sc1C(=O)O] | 3-[(2,4-Dichloro-benzoyl)-isopropyl-amino]- 5-(3,3-dimethyl-but-1-ynyl)-thiophene-2- carboxylic acid |
| 2 | 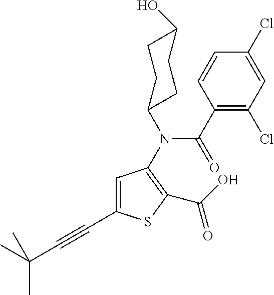 [SMILES: CC(C)(C)C#Cc1cc(N(C(=O)c2ccc(Cl)cc2Cl)C2CCC(O)CC2)c(C(=O)O)s1] | 3-[(2,4-Dichloro-benzoyl)-(trans-4-hydroxy- cyclohexyl)-amino]-5-(3,3-dimethyl-but-1- ynyl)-thiophene-2-carboxylic acid |
| 3 | 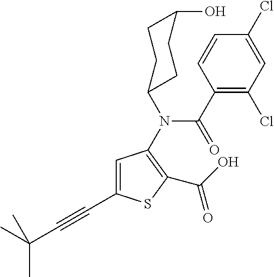 [SMILES: CC(C)(C)C#Cc1cc(N(C(=O)c2ccc(Cl)cc2Cl)C2CCC(O)CC2)c(C(=O)O)s1] | 3-[(2,4-Dichloro-benzoyl)-(cis-4-hydroxy- cyclohexyl)-amino]-5-(3,3-dimethyl-but-1- ynyl)-thiophene-2-carboxylic acid |
| 4 | 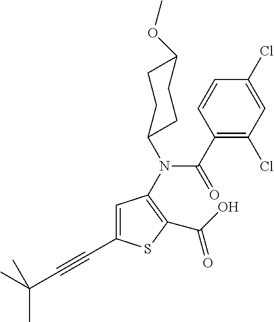 [SMILES: COC1CCC(N(C(=O)c2ccc(Cl)cc2Cl)c2cc(C#CC(C)(C)C)sc2C(=O)O)CC1] | 3-[(2,4-Dichloro-benzoyl)-(trans-4-methoxy- cyclohexyl)-amino]-5-(3,3-dimethyl-but-1- ynyl)-thiophene-2-carboxylic acid |
| 5 | 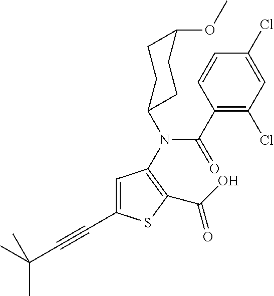 [SMILES: COC1CCC(N(C(=O)c2ccc(Cl)cc2Cl)c2cc(C#CC(C)(C)C)sc2C(=O)O)CC1] | 3-[(2,4-Dichloro-benzoyl)-(cis-4-methoxy- cyclohexyl)-amino]-5-(3,3-dimethyl-but-1- ynyl)-thiophene-2-carboxylic acid |
| 6 | 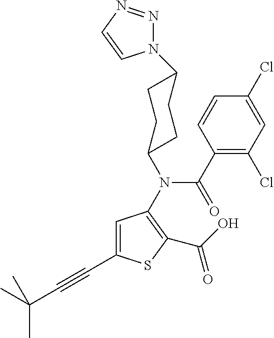 [SMILES: CC(C)(C)C#Cc1cc(N(C(=O)c2ccc(Cl)cc2Cl)C2CCC(n3ccnn3)CC2)c(C(=O)O)s1] | 3-[(2,4-Dichloro-benzoyl)-(trans-4- [1,2,3]triazol-1-yl-cyclohexyl)-amino]-5- (3,3-dimethyl-but-1-ynyl)-thiophene-2- carboxylic acid |
| 7 | 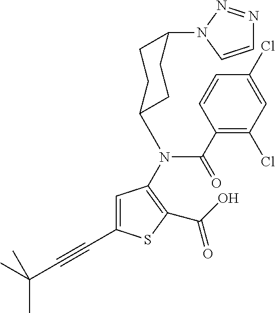 [SMILES: CC(C)(C)C#Cc1cc(N(C(=O)c2ccc(Cl)cc2Cl)C2CCC(n3ccnn3)CC2)c(C(=O)O)s1] | 3-[(2,4-Dichloro-benzoyl)-(cis-4- [1,2,3]triazol-1-yl-cyclohexyl)-amino]-5- (3,3-dimethyl-but-1-ynyl)-thiophene-2- carboxylic acid |
| 8 | 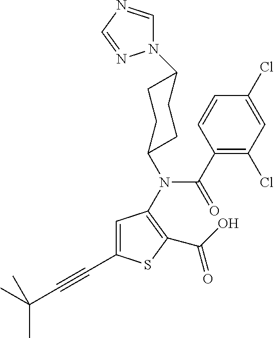 [SMILES: CC(C)(C)C#Cc1cc(N(C(=O)c2ccc(Cl)cc2Cl)C2CCC(n3cncn3)CC2)c(C(=O)O)s1] | 3-[(2,4-Dichloro-benzoyl)-(trans-4- [1,2,4]triazol-1-yl-cyclohexyl)-amino]-5- (3,3-dimethyl-but-1-ynyl)-thiophene-2- carboxylic acid |
| 9 | 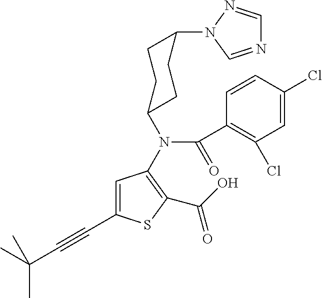 [SMILES: CC(C)(C)C#Cc1cc(N(C(=O)c2ccc(Cl)cc2Cl)C2CCC(n3cncn3)CC2)c(C(=O)O)s1] | 3-[(2,4-Dichloro-benzoyl)-(cis-4- [1,2,4]triazol-1-yl-cyclohexyl)-amino]-5- (3,3-dimethyl-but-1-ynyl)-thiophene-2- carboxylic acid |
| 10 | 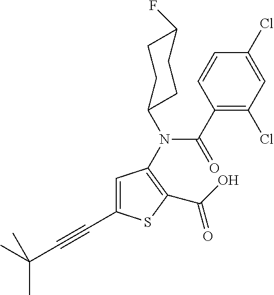 [SMILES: CC(C)(C)C#Cc1cc(N(C(=O)c2ccc(Cl)cc2Cl)C2CCC(F)CC2)c(C(=O)O)s1] | 3-[(2,4-Dichloro-benzoyl)-(trans-4-fluoro- cyclohexyl)-amino]-5-(3,3-dimethyl-but-1- ynyl)-thiophene-2-carboxylic acid |
| 11 | 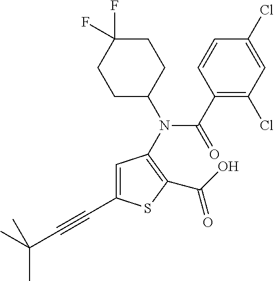 [SMILES: CC(C)(C)C#Cc1cc(N(C(=O)c2ccc(Cl)cc2Cl)C2CCC(F)(F)CC2)c(C(=O)O)s1] | 3-[(2,4-Dichloro-benzoyl)-(4,4-difluoro- cyclohexyl)-amino]-5-(3,3-dimethyl-but-1- ynyl)-thiophene-2-carboxylic acid |
| 12 | 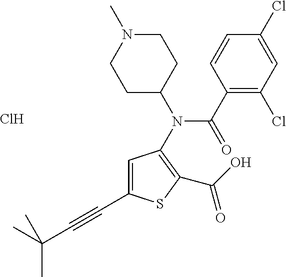 [SMILES: Cl ; CN1CCC(N(C(=O)c2ccc(Cl)cc2Cl)c2cc(C#CC(C)(C)C)sc2C(=O)O)CC1] | 3-[(2,4-Dichloro-benzoyl)-(1-methyl- piperidin-4-yl)-amino]-5-(3,3-dimethyl-but- 1-ynyl)-thiophene-2-carboxylic acid hydrochloride |
| 13 | 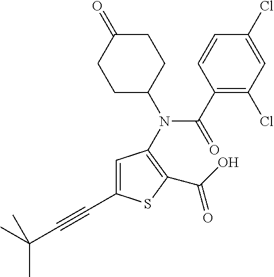 [SMILES: CC(C)(C)C#Cc1cc(N(C(=O)c2ccc(Cl)cc2Cl)C2CCC(=O)CC2)c(C(=O)O)s1] | 3-[(2,4-Dichloro-benzoyl)-(4-oxo- cyclohexyl)-amino]-5-(3,3-dimethyl-but-1- ynyl)-thiophene-2-carboxylic acid |
| 14 | 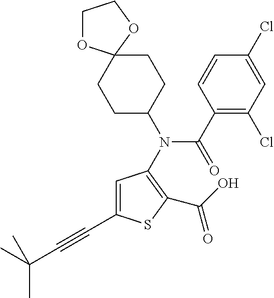 [SMILES: CC(C)(C)C#Cc1cc(N(C(=O)c2ccc(Cl)cc2Cl)C2CCC3(CC2)OCCO3)c(C(=O)O)s1] | 3-[(2,4-Dichloro-benzoyl)-(1,4-dioxa- spiro[4.5]dec-8-yl)-amino]-5-(3,3-dimethyl- but-1-ynyl)-thiophene-2-carboxylic acid |
| 15 | 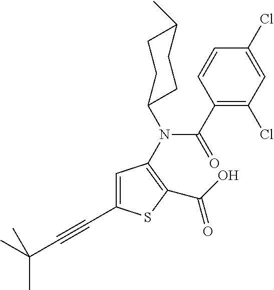 [SMILES: CC1CCC(N(C(=O)c2ccc(Cl)cc2Cl)c2cc(C#CC(C)(C)C)sc2C(=O)O)CC1] | 3-[(2,4-Dichloro-benzoyl)-(trans-4-methyl- cyclohexyl)-amino]-5-(3,3-dimethyl-but-1- ynyl)-thiophene-2-carboxylic acid |
| 16 | 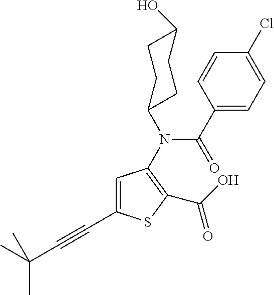 [SMILES: CC(C)(C)C#Cc1cc(N(C(=O)c2ccc(Cl)cc2)C2CCC(O)CC2)c(C(=O)O)s1] | 3-[(4-Chloro-benzoyl)-(trans-4-hydroxy- cyclohexyl)-amino]-5-(3,3-dimethyl-but-1- ynyl)-thiophene-2-carboxylic acid |
| 17 | 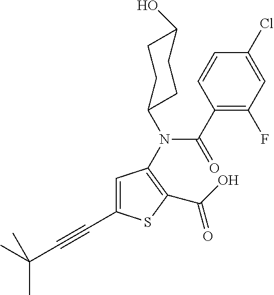 [SMILES: CC(C)(C)C#Cc1cc(N(C(=O)c2ccc(Cl)cc2F)C2CCC(O)CC2)c(C(=O)O)s1] | 3-[(4-Chloro-2-fluoro-benzoyl)-(trans-4- hydroxy-cyclohexyl)-amino]-5-(3,3- dimethyl-but-1-ynyl)-thiophene-2- carboxylic acid |
| 18 | 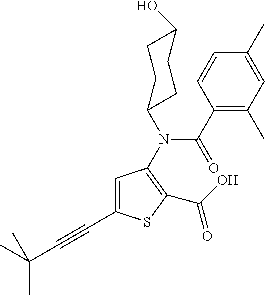 [SMILES: Cc1ccc(C(=O)N(c2cc(C#CC(C)(C)C)sc2C(=O)O)C2CCC(O)CC2)c(C)c1] | 3-[(2,4-Dimethyl-benzoyl)-(trans-4-hydroxy- cyclohexyl)-amino]-5-(3,3-dimethyl-but-1- ynyl)-thiophene-2-carboxylic acid |
| 19 | 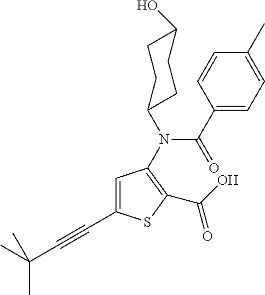 [SMILES: Cc1ccc(C(=O)N(c2cc(C#CC(C)(C)C)sc2C(=O)O)C2CCC(O)CC2)cc1] | 5-(3,3-Dimethyl-but-1-ynyl)-3-[(trans-4- hydroxy-cyclohexyl)-(4-methyl-benzoyl)- amino]-thiophene-2-carboxylic acid |
| 20 | 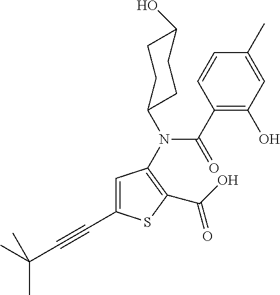 [SMILES: Cc1ccc(C(=O)N(c2cc(C#CC(C)(C)C)sc2C(=O)O)C2CCC(O)CC2)c(O)c1] | 5-(3,3-Dimethyl-but-1-ynyl)-3-[(trans-4- hydroxy-cyclohexyl)-(2-hydroxy-4-methyl- benzoyl)-amino]-thiophene-2-carboxylic acid |
| 21 | 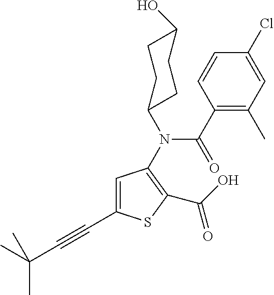 [SMILES: Cc1cc(Cl)ccc1C(=O)N(c1cc(C#CC(C)(C)C)sc1C(=O)O)C1CCC(O)CC1] | 3-[(4-Chloro-2-methyl-benzoyl)-(trans-4- hydroxy-cyclohexyl)-amino]-5-(3,3- dimethyl-but-1-ynyl)-thiophene-2- carboxylic acid |
| 22 | 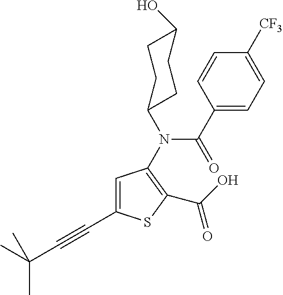 [SMILES: CC(C)(C)C#Cc1cc(N(C(=O)c2ccc(C(F)(F)F)cc2)C2CCC(O)CC2)c(C(=O)O)s1] | 5-(3,3-Dimethyl-but-1-ynyl)-3-[(trans-4- hydroxy-cyclohexyl)-(4-trifluoromethyl- benzoyl)-amino]-thiophene-2-carboxylic acid) |
| 23 | 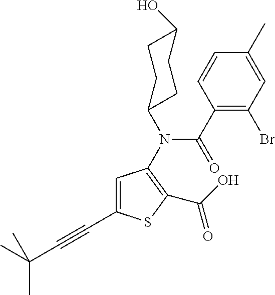 [SMILES: Cc1ccc(C(=O)N(c2cc(C#CC(C)(C)C)sc2C(=O)O)C2CCC(O)CC2)c(Br)c1] | 3-[(2-Bromo-4-methyl-benzoyl)-(trans-4- hydroxy-cyclohexyl)-amino]-5-(3,3- dimethyl-but-1-ynyl)-thiophene-2- carboxylic acid |
| 24 | 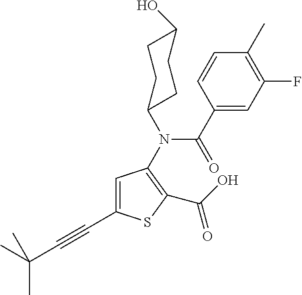 [SMILES: Cc1ccc(C(=O)N(c2cc(C#CC(C)(C)C)sc2C(=O)O)C2CCC(O)CC2)cc1F] | 5-(3,3-Dimethyl-but-1-ynyl)-3-[(3-fluoro-4- methyl-benzoyl)-(trans-4-hydroxy- cyclohexyl)-amino]-thiophene-2-carboxylic acid |
| 25 | 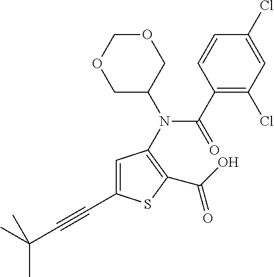 [SMILES: CC(C)(C)C#Cc1cc(N(C(=O)c2ccc(Cl)cc2Cl)C2COCOC2)c(C(=O)O)s1] | 3-[(2,4-Dichloro-benzoyl)-[1,3]dioxan-5-yl- amino]-5-(3,3-dimethyl-but-1-ynyl)- thiophene-2-carboxylic acid |
| 26 | 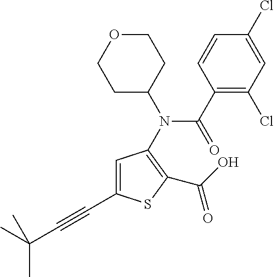 [SMILES: CC(C)(C)C#Cc1cc(N(C(=O)c2ccc(Cl)cc2Cl)C2CCOCC2)c(C(=O)O)s1] | 3-[(2,4-Dichloro-benzoyl)-(tetrahydro- pyran-4-yl)-amino]-5-(3,3-dimethyl-but-1- ynyl)-thiophene-2-carboxylic acid |
| 27 | 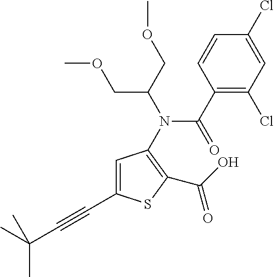 [SMILES: COCC(COC)N(C(=O)c1ccc(Cl)cc1Cl)c1cc(C#CC(C)(C)C)sc1C(=O)O] | 3-[(2,4-Dichloro-benzoyl)-(2-methoxy-1- methoxymethyl-ethyl)-amino]-5-(3,3- dimethyl-but-1-ynyl)-thiophene-2- carboxylic acid |
| 28 | 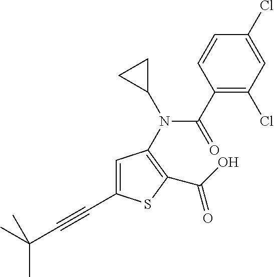 [SMILES: CC(C)(C)C#Cc1cc(N(C(=O)c2ccc(Cl)cc2Cl)C2CC2)c(C(=O)O)s1] | 3-[Cyclopropyl-(2,4-dichloro-benzoyl)- amino]-5-(3,3-dimethyl-but-1-ynyl)- thiophene-2-carboxylic acid |
| 29 | 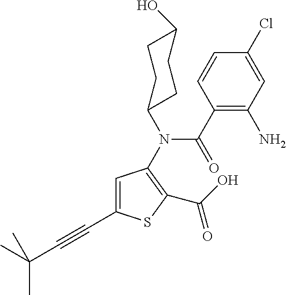 [SMILES: CC(C)(C)C#Cc1cc(N(C(=O)c2ccc(Cl)cc2N)C2CCC(O)CC2)c(C(=O)O)s1] | 3-[(2-Amino-4-chloro-benzoyl)-(trans-4- hydroxy-cyclohexyl)-amino]-5-(3,3- dimethyl-but-1-ynyl)-thiophene-2- carboxylic acid |
| 30 | 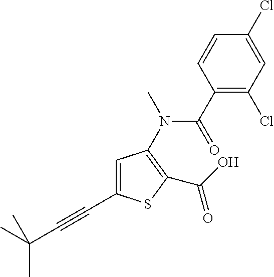 [SMILES: CN(C(=O)c1ccc(Cl)cc1Cl)c1cc(C#CC(C)(C)C)sc1C(=O)O] | 3-[(2,4-Dichloro-benzoyl)-methyl-amino]- 5-(3,3-dimethyl-but-1-ynyl)-thiophene-2- carboxylic acid |
| 31 | 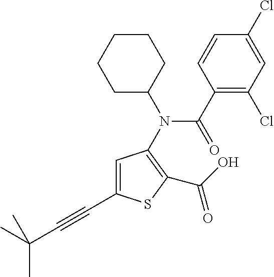 [SMILES: CC(C)(C)C#Cc1cc(N(C(=O)c2ccc(Cl)cc2Cl)C2CCCCC2)c(C(=O)O)s1] | 3-[Cyclohexyl-(2,4-dichloro-benzoyl)- amino]-5-(3,3-dimethyl-but-1-ynyl)- thiophene-2-carboxylic acid |
| 32 | 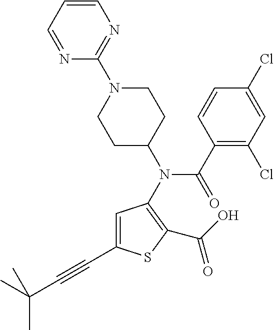 [SMILES: CC(C)(C)C#Cc1cc(N(C(=O)c2ccc(Cl)cc2Cl)C2CCN(c3ncccn3)CC2)c(C(=O)O)s1] | 3-[(2,4-Dichloro-benzoyl)-(1-pyrimidin-2-yl- piperidin-4-yl)-amino]-5-(3,3-dimethyl-but- 1-ynyl)-thiophene-2-carboxylic acid |
| 33 | 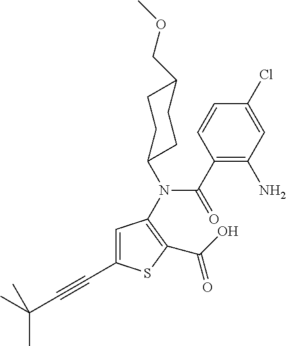 [SMILES: COCC1CCC(N(C(=O)c2ccc(Cl)cc2N)c2cc(C#CC(C)(C)C)sc2C(=O)O)CC1] | 3-[(2-Amino-4-chloro-benzoyl)-(trans-4- methoxymethyl-cyclohexyl)-amino]-5-(3,3- dimethyl-but-1-ynyl)-thiophene-2- carboxylic acid |
| 34 | 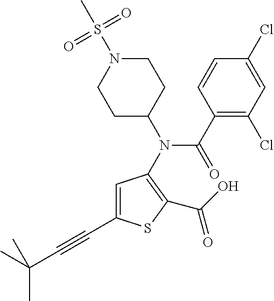 [SMILES: CC(C)(C)C#Cc1cc(N(C(=O)c2ccc(Cl)cc2Cl)C2CCN(S(C)(=O)=O)CC2)c(C(=O)O)s1] | 3-[(2,4-Dichloro-benzoyl)-(1- methanesulfonyl-piperidin-4-yl)-amino]-5- 3,3-dimethyl-but-1-ynyl)-thiophene-2- carboxylic acid |
| 35 | 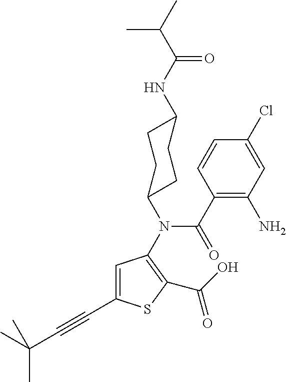 [SMILES: CC(C)C(=O)NC1CCC(N(C(=O)c2ccc(Cl)cc2N)c2cc(C#CC(C)(C)C)sc2C(=O)O)CC1] | 3-[(2-Amino-4-chloro-benzoyl)-(trans-4- isobutyrylamino-cyclohexyl)-amino]-5-(3,3- dimethyl-but-1-ynyl)-thiophene-2- carboxylic acid |
| 36 | 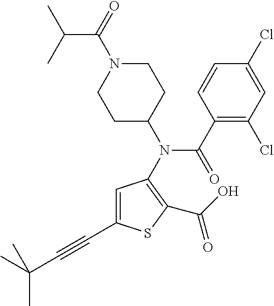 [SMILES: CC(C)C(=O)N1CCC(N(C(=O)c2ccc(Cl)cc2Cl)c2cc(C#CC(C)(C)C)sc2C(=O)O)CC1] | 3-[(2,4-Dichloro-benzoyl)-(1-isobutyryl- piperidin-4-yl)-amino]-5-(3,3-dimethyl-but- 1-ynyl)-thiophene-2-carboxylic acid |
| 37 | 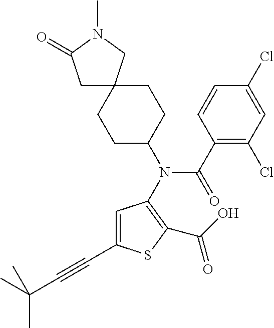 [SMILES: CN1CC2(CCC(N(C(=O)c3ccc(Cl)cc3Cl)c3cc(C#CC(C)(C)C)sc3C(=O)O)CC2)CC1=O] | 3-[(2,4-Dichloro-benzoyl)-(2-methyl-3-oxo- 2-aza-spiro[4.5]dec-8-yl)-amino]-5-(3,3- dimethyl-but-1-ynyl)-thiophene-2- carboxylic acid |
| 38 | 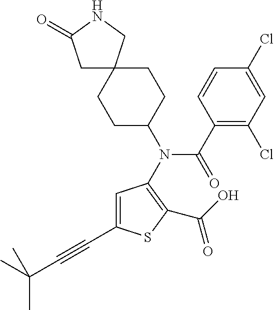 [SMILES: CC(C)(C)C#Cc1cc(N(C(=O)c2ccc(Cl)cc2Cl)C2CCC3(CC2)CNC(=O)C3)c(C(=O)O)s1] | 3-[(2,4-Dichloro-benzoyl)-(3-oxo-2-aza- spiro[4.5]dec-8-yl)-amino]-5-(3,3-dimethyl- but-1-ynyl)-thiophene-2-carboxylic acid |
| 39 | 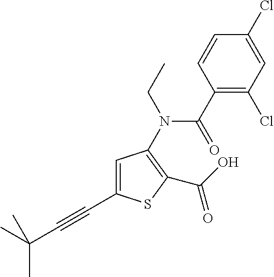 [SMILES: CCN(C(=O)c1ccc(Cl)cc1Cl)c1cc(C#CC(C)(C)C)sc1C(=O)O] | 3-[(2,4-Dichloro-benzoyl)-ethyl-amino]-5- (3,3-dimethyl-but-1-ynyl)-thiophene-2- carboxylic acid |
| 40 | 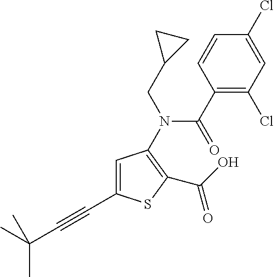 [SMILES: CC(C)(C)C#Cc1cc(N(CC2CC2)C(=O)c2ccc(Cl)cc2Cl)c(C(=O)O)s1] | 3-[Cyclopropylmethyl-(2,4-dichloro- benzoyl)-amino]-5-(3,3-dimethyl-but-1- ynyl)-thiophene-2-carboxylic acid |
| 41 | 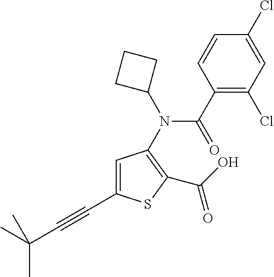 [SMILES: CC(C)(C)C#Cc1cc(N(C(=O)c2ccc(Cl)cc2Cl)C2CCC2)c(C(=O)O)s1] | 3-[Cyclobutyl-(2,4-dichloro-benzoyl)- amino]-5-(3,3-dimethyl-but-1-ynyl)- thiophene-2-carboxylic acid |
| 42 | 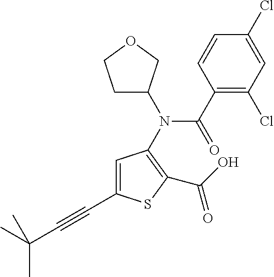 [SMILES: CC(C)(C)C#Cc1cc(N(C(=O)c2ccc(Cl)cc2Cl)C2CCOC2)c(C(=O)O)s1] | 3-[(2,4-Dichloro-benzoyl)-(tetrahydro- furan-3-yl)-amino]-5-(3,3-dimethyl-but-1- ynyl)-thiophene-2-carboxylic acid |
| 43 | 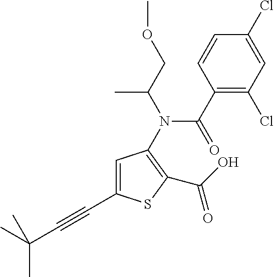 [SMILES: COCC(C)N(C(=O)c1ccc(Cl)cc1Cl)c1cc(C#CC(C)(C)C)sc1C(=O)O] | 3-[(2,4-Dichloro-benzoyl)-(2-methoxy-1- methyl-ethyl)-amino]-5-(3,3-dimethyl-but- 1-ynyl)-thiophene-2-carboxylic acid |
| 44 | 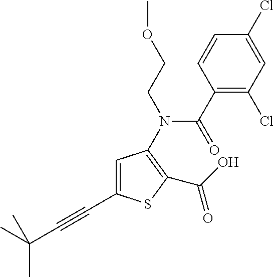 [SMILES: COCCN(C(=O)c1ccc(Cl)cc1Cl)c1cc(C#CC(C)(C)C)sc1C(=O)O] | 3-[(2,4-Dichloro-benzoyl)-(2-methoxy- ethyl)-amino]-5-(3,3-dimethyl-but-1-ynyl)- thiophene-2-carboxylic acid |
| 45 | 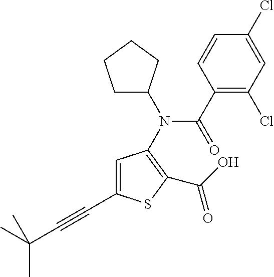 [SMILES: CC(C)(C)C#Cc1cc(N(C(=O)c2ccc(Cl)cc2Cl)C2CCCC2)c(C(=O)O)s1] | 3-[Cyclopentyl-(2,4-dichloro-benzoyl)- amino]-5-(3,3-dimethyl-but-1-ynyl)- thiophene-2-carboxylic acid |
| 46 | 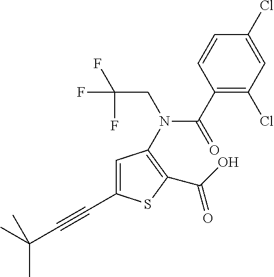 [SMILES: CC(C)(C)C#Cc1cc(N(CC(F)(F)F)C(=O)c2ccc(Cl)cc2Cl)c(C(=O)O)s1] | 3-[(2,4-Dichloro-benzoyl)-(2,2,2-trifluoro- ethyl)-amino]-5-(3,3-dimethyl-but-1-ynyl)- thiophene-2-carboxylic acid |
| 47 | 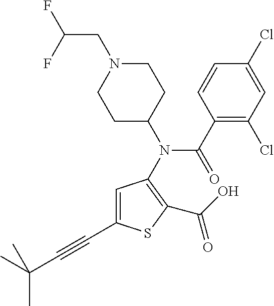 [SMILES: CC(C)(C)C#Cc1cc(N(C(=O)c2ccc(Cl)cc2Cl)C2CCN(CC(F)F)CC2)c(C(=O)O)s1] | 3-{(2,4-Dichloro-benzoyl)-[1-(2,2-difluoro- ethyl)-piperidin-4-yl]-amino}-5-(3,3- dimethyl-but-1-ynyl)-thiophene-2- carboxylic acid |
| 48 | 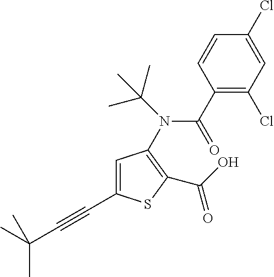 [SMILES: CC(C)(C)C#Cc1cc(N(C(=O)c2ccc(Cl)cc2Cl)C(C)(C)C)c(C(=O)O)s1] | 3-[tert-Butyl-(2,4-dichloro-benzoyl)-amino]- 5-(3,3-dimethyl-but-1-ynyl)-thiophene-2- carboxylic acid |
| 49 | 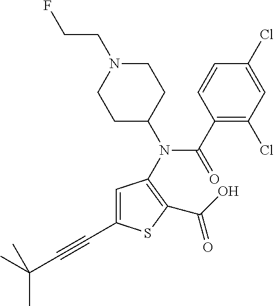 [SMILES: CC(C)(C)C#Cc1cc(N(C(=O)c2ccc(Cl)cc2Cl)C2CCN(CCF)CC2)c(C(=O)O)s1] | 3-{(2,4-Dichloro-benzoyl)-[1-(2-fluoro- ethyl)-piperidin-4-yl]-amino}-5-(3,3- dimethyl-but-1-ynyl)-thiophene-2- carboxylic acid |
| 50 | 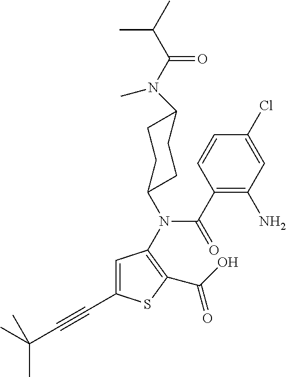 [SMILES: CC(C)C(=O)N(C)C1CCC(N(C(=O)c2ccc(Cl)cc2N)c2cc(C#CC(C)(C)C)sc2C(=O)O)CC1] | 3-{(2-Amino-4-chloro-benzoyl)-[4- (isobutyryl-methyl-amino)-cyclohexyl]- amino}-5-(3,3-dimethyl-but-1-ynyl)- thiophene-2-carboxylic acid |
| 51 | 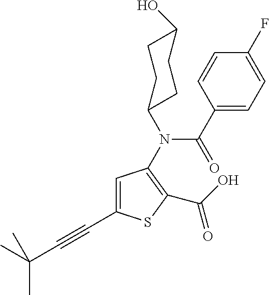 [SMILES: CC(C)(C)C#Cc1cc(N(C(=O)c2ccc(F)cc2)C2CCC(O)CC2)c(C(=O)O)s1] | 5-(3,3-Dimethyl-but-1-ynyl)-3-[(4-fluoro- benzoyl)-(4-hydroxy-cyclohexyl)-amino]- thiophene-2-carboxylic acid |
| 52 | 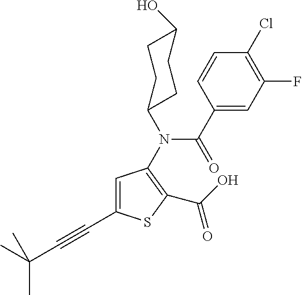 [SMILES: CC(C)(C)C#Cc1cc(N(C(=O)c2ccc(Cl)c(F)c2)C2CCC(O)CC2)c(C(=O)O)s1] | 3-[(4-Chloro-3-fluoro-benzoyl)-(4-hydroxy- cyclohexyl)-amino]-5-(3,3-dimethyl-but-1- ynyl)-thiophene-2-carboxylic acid |
| 53 | 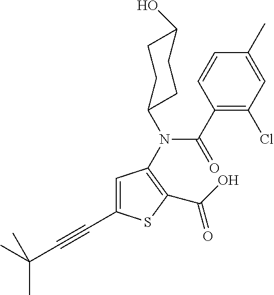 [SMILES: Cc1ccc(C(=O)N(c2cc(C#CC(C)(C)C)sc2C(=O)O)C2CCC(O)CC2)c(Cl)c1] | 3-[(2-Chloro-4-methyl-benzoyl)-(4-hydroxy- cyclohexyl)-amino]-5-(3,3-dimethyl-but-1- ynyl)-thiophene-2-carboxylic acid |
| 54 | 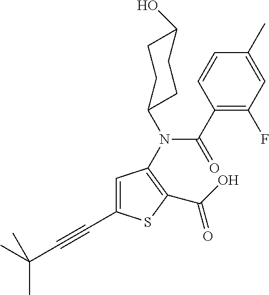 [SMILES: Cc1ccc(C(=O)N(c2cc(C#CC(C)(C)C)sc2C(=O)O)C2CCC(O)CC2)c(F)c1] | 5-(3,3-Dimethyl-but-1-ynyl)-3-[(2-fluoro-4- methyl-benzoyl)-(4-hydroxy-cyclohexyl)- amino]-thiophene-2-carboxylic acid |

and pharmaceutically acceptable salts thereof.

According to another aspect of the invention, the compounds representative of the invention are selected from the compounds listed in Table 2 or pharmaceutically acceptable salts thereof.

According to another aspect of the invention, the compounds representative of the invention are selected from any one of the following structural formulae or pharmaceutically acceptable salts thereof:

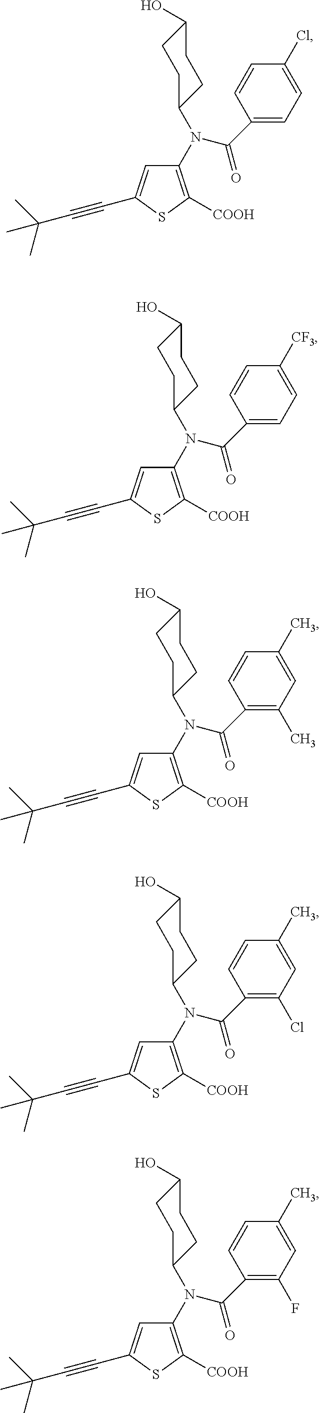

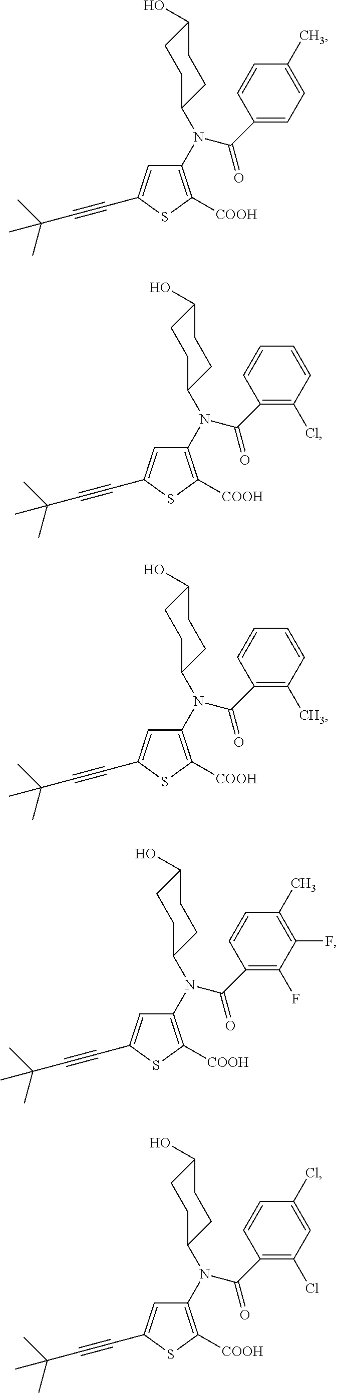 [SMILES: Cc1ccc(C(=O)N(c2cc(C#CC(C)(C)C)sc2C(=O)O)C2CCC(O)CC2)cc1 ; CC(C)(C)C#Cc1cc(N(C(=O)c2ccccc2Cl)C2CCC(O)CC2)c(C(=O)O)s1 ; Cc1ccccc1C(=O)N(c1cc(C#CC(C)(C)C)sc1C(=O)O)C1CCC(O)CC1 ; Cc1ccc(C(=O)N(c2cc(C#CC(C)(C)C)sc2C(=O)O)C2CCC(O)CC2)c(F)c1F ; CC(C)(C)C#Cc1cc(N(C(=O)c2ccc(Cl)cc2Cl)C2CCC(O)CC2)c(C(=O)O)s1]

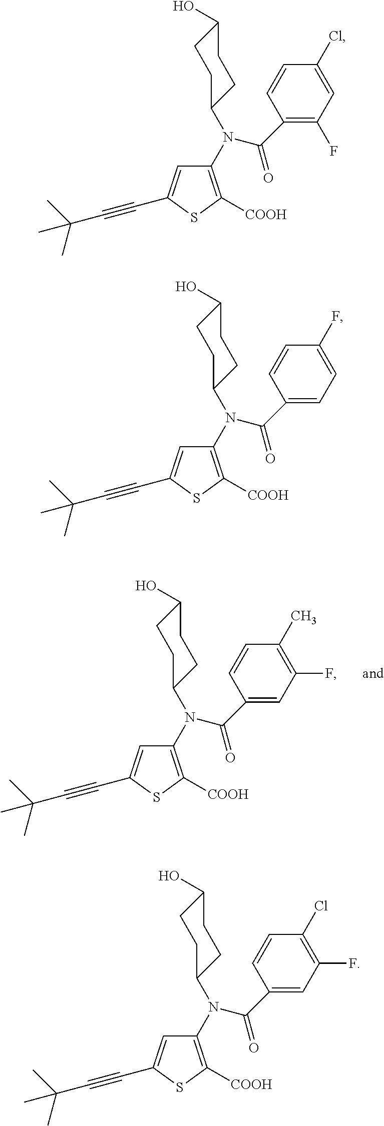 [SMILES: CC(C)(C)C#Cc1cc(N(C(=O)c2ccc(Cl)cc2F)C2CCC(O)CC2)c(C(=O)O)s1 ; CC(C)(C)C#Cc1cc(N(C(=O)c2ccc(F)cc2)C2CCC(O)CC2)c(C(=O)O)s1 ; Cc1ccc(C(=O)N(c2cc(C#CC(C)(C)C)sc2C(=O)O)C2CCC(O)CC2)cc1F ; CC(C)(C)C#Cc1cc(N(C(=O)c2ccc(Cl)c(F)c2)C2CCC(O)CC2)c(C(=O)O)s1]

According to another aspect of the invention, the compounds representative of the invention are selected from any one of the following structural formulae or pharmaceutically acceptable salts thereof:

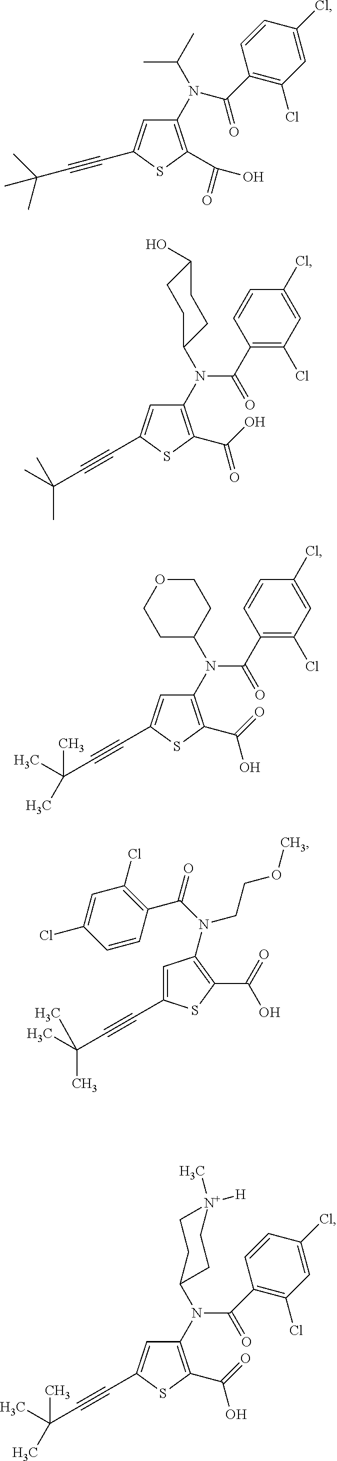 [SMILES: CC(C)N(C(=O)c1ccc(Cl)cc1Cl)c1cc(C#CC(C)(C)C)sc1C(=O)O ; CC(C)(C)C#Cc1cc(N(C(=O)c2ccc(Cl)cc2Cl)C2CCC(O)CC2)c(C(=O)O)s1 ; CC(C)(C)C#Cc1cc(N(C(=O)c2ccc(Cl)cc2Cl)C2CCOCC2)c(C(=O)O)s1 ; COCCN(C(=O)c1ccc(Cl)cc1Cl)c1cc(C#CC(C)(C)C)sc1C(=O)O ; C[NH+]1CCC(N(C(=O)c2ccc(Cl)cc2Cl)c2cc(C#CC(C)(C)C)sc2C(=O)O)CC1]

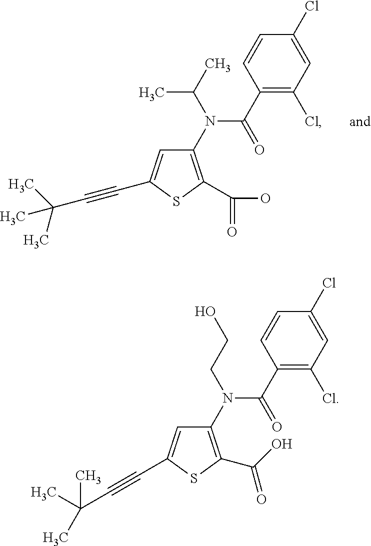 [SMILES: CC(C)N(C(=O)c1ccc(Cl)cc1Cl)c1cc(C#CC(C)(C)C)sc1C(=O)O ; CC(C)(C)C#Cc1cc(N(CCO)C(=O)c2ccc(Cl)cc2Cl)c(C(=O)O)s1]

According to another aspect of the invention, the compounds representative of the invention are selected from any one of the structural formulae depicted in Table 2 or pharmaceutically acceptable salts thereof.

A compound of formula (I) may be prepared by reacting a compound of formula (II):

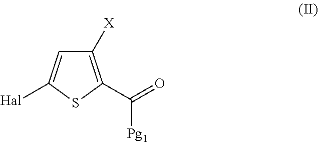

with a compound of the formula:

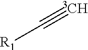

under conventional Sonogashira coupling conditions;

wherein;

X is as defined above, for example, —NR₃—CO—R₂,

R₁, R₂ and R₃ are as defined herein,

Pg₁ is OH or a carboxyl protecting group, and

Hal is Cl, Br, or I (e.g., Br).

In a further embodiment, Pg₁ is methoxy.

The Sonogashira coupling reaction is a well established method for producing acetylene containing compounds. Conditions for such coupling are well known in the art and can be found for example in the examples of the present application in Yamaguchi et al (Synlett 1999, No. 5, 549-550) or in Tykwinski et al, Angew. Chem. Int. Ed. 2003, 42, 1566-1568.

In another aspect, there is provided a pharmaceutical composition comprising at least one compound of the invention and at least one pharmaceutically acceptable carrier or excipient.

In one embodiment, the present invention provides a pharmaceutical combination comprising at least one compound according to the invention described herein, and further comprising administering at least one additional agent.

In another aspect, there is provided a combination comprising a compound of the invention and one or more additional agents chosen from viral serine protease inhibitors, viral polymerase inhibitors, viral helicase inhibitors, immunomudulating agents, antiviral agents, antioxidant agents, antibacterial agents, therapeutic vaccines, hepatoprotectant agents, antisense agent, inhibitors of HCV NS2/3 protease and inhibitors of internal ribosome entry site (IRES).

In another aspect, there is provided a method for treating or preventing a Flaviviridae viral infection in a patient comprising administering to the patient a therapeutically effective amount of a compound, composition or combination of the invention.

In another aspect, there is provided a method for treating or preventing an HCV viral infection in a patient comprising administering to the patient a therapeutically effective amount of a compound, composition or combination of the invention.

In another aspect, there is provided a method for inhibiting or reducing the activity of viral polymerase in a patient comprising administering to the patient a therapeutically effective amount of a compound, composition or combination of the invention.

In a further aspect, there is provided the use of a compound, composition or combination of the invention for treating or preventing a Flaviviridae viral infection in a patient.

In a further aspect, there is provided the use of a compound, composition or combination of the invention for treating or preventing an HCV viral infection in a patient.

In still another aspect, there is provided the use of a compound, composition or combination of the invention for inhibiting or reducing the activity of viral polymerase in a patient.

In still another aspect, there is provided the use of a compound, composition or combination of the invention for the manufacture of a medicament for treating or preventing a viral Flaviridae infection in a patient.

In still another aspect, there is provided the use of a compound, composition or combination of the invention for the manufacture of a medicament for treating or preventing an HCV viral infection in a patient.

In one embodiment, the present invention provides the use of a compound according to the invention described herein as an anti-HCV agent.

In one embodiment, the present invention provides a method for treating or preventing a Flaviviridae viral infection in a host comprising administering to the host a therapeutically effective amount of at least one compound according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

In one embodiment, the viral infection is chosen from Flavivirus infections.

In one embodiment, the Flavivirus infection is Hepatitis C virus (HCV), bovine viral diarrhea virus (BVDV), hog cholera virus, dengue fever virus, Japanese encephalitis virus or yellow fever virus.

In one embodiment, the Flaviviridea viral infection is hepatitis C viral infection (HCV).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in:

WO 03/010140 (Boehringer Ingelheim), WO 03/026587 (Bristol Myers Squibb); WO 02/100846 A1, WO 02/100851 A2, WO 01/85172 A1 (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121 A2 (Idenix), WO 02/069903 A2 (Biocryst Pharmaceuticals Inc.), and WO 02/057287 A2 (Merck/Isis) and WO 02/057425 A2 (Merck/Isis).

Specific examples of inhibitors of an HCV polymerase, include VCH-759 (ViroChem Pharma), VCH-916 (ViroChem Pharma), VCH-222 (ViroChem Pharma), R1626 (Roche), R7128 (Roche/Pharmasset), PF-868554 (Pfizer), MK-0608 (Merck/Isis), MK-3281 (Merck), A-837093 (Abbott), GS 9190 (Gilead), ana598 (Anadys), HCV-796 (Viropharma) and GSK625433 (GlaxoSmithKline).

The term “viral helicase inhibitors” as used herein means an agent that is effective to inhibit the function of a viral helicase including a flaviviridae helicase in a mammal.

“Immunomodulatory agent” as used herein means those agents that are effective to enhance or potentiate the immune system response in a mammal.

Immunomodulatory agents include, for example, class I interferons (such as α-, β-, δ- and Ω-interferons, τ-interferons, consensus interferons and asialo-interferons), class II interferons (such as γ-interferons) and pegylated interferons.

The term “class I interferon” as used herein means an interferon selected from a group of interferons that all bind to receptor type 1. This includes both naturally and synthetically produced class I interferons. Examples of class I interferons include α-, β-, δ- and Ω-interferons, τ-interferons, consensus interferons and asialo-interferons. The term “class II interferon” as used herein means an interferon selected from a group of interferons that all bind to receptor type II. Examples of class II interferons include γ-interferons.

Specific examples of Immunomodulatory agent as used herein include IL-29 (PEG-Interferon Lambda, ZymoGenetics), Belerofon (Nautilus Biotech) injectable or oral, Oral Interferon alpha (Amarillo Biosciences), BLX-883 (Locteron, Biolex Therapeutics/Octoplus), Omega Interferon (Intarcia Therapeutics), multiferon (Viragen), Albuferon (Human Genome Sciences), consensus Interferon (Infergen, Three Rivers Pharmaceuticals), Medusa Interferon (Flamel Technologies), NOV-205 (Novelos Therapeutics), Oglufanide disodium (Implicit Bioscience), SCV-07 (SciClone), Zadaxin® (thymalfasin, SciClone/Sigma-Tau), AB68 (XTL bio) and Civacir (NABI).

The term “viral serine protease inhibitor” as used herein means an agent that is effective to inhibit the function of the viral serine protease including HCV serine protease in a mammal. Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

Specific examples of viral serine protease inhibitors include Telaprevir (VX-950, Vertex), VX-500 (Vertex), TMC435350 (Tibotec/Medivir), MK-7009 (Merck), ITMN-191 (R7227, InterMune/Roche) and Boceprevir (SCH503034, Schering).

Inhibitor internal ribosome entry site (IRES) include those compounds is described in WO 2006019831 (PTC therapeutics).

In one embodiment, the additional agent is interferon α, ribavirin, and silybum marianum.

In one embodiment, the additional agent is interferon α 1A, interferon α 1B, interferon α 2A (Roferon), PEG-interferon α 2A (Pegasys), interferon α 2B (Intron A) or PEG-interferon α 2B (Peg-Intron).

In one embodiment, the additional agents are standard or pegylated interferon α (Roferon, Pegasys, Intron A, Peg-Intron) in combination with ribavirin.

In one embodiment, the additional agent is chosen from A-831 (AZD0530, Arrow Therapeutics acquired by AstraZeneca), TLR9 agonist: IMO-2125 (Idera Pharmaceuticals), PYN17 (Phynova), Vavituximab (Tarvacin, Peregrine), DEBIO-025 (DEBIO), NIM-811 (Novartis), SCY635 (Scynexis), PF-03491390 (IDN-6556, Pfizer), Suvus (formerly BIVN-401, Virostat, Bioenvision), MX-3253 (Celgosivir, Migenix), Viramidine (Taribavirin, Valeant Pharmaceuticals), Hepaconda (Giaconda), TT033 (Benitec/Tacere Bio/Pfizer), SIRNA-034 (Sirna Therapeutics acquired by Merck) and EHC-18 (Enzo Biochem), ACH-1095 (Achillion/Gilead), JKB-022 (Jenkin), CTS-1027 (Conatus), MitoQ (mitoquinone, Antipodean Pharmaceuticals), Alinia (nitazoxanide, Romark Laboratories) and Bavituximab (Peregrine Pharm).

In one embodiment, the additional agent is a therapeutic vaccine chosen from CSL123 (Chiron/CSL), IC41 (Intercell Novartis), GI 5005 (Globeimmune), TG4040 (Transgene), Chronvac C (Tripep/Inovio), GNI-103 (GENimmune), HCV/MF59 (Chiron/Novartis), PeviPRO™ (Pevion biotect).

The combinations referred to above may conveniently be presented for use in the form of a pharmaceutical formulation and thus pharmaceutical formulations comprising a combination as defined above together with a pharmaceutically acceptable carrier therefor comprise a further aspect of the invention.

The individual components for use in the method of the present invention or combinations of the present invention may be administered either sequentially or simultaneously in separate or combined pharmaceutical formulations.

In one combination embodiment, the compound and additional agent are administered sequentially.

In another combination embodiment, the compound and additional agent are administered simultaneously.

It will be appreciated by those skilled in the art that the compounds in accordance with the present invention can exists as stereoisomers (for example, optical (+ and −), geometrical (cis and trans) and conformational isomers (axial and equatorial). All such stereoisomers are included in the scope of the present invention.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds of the present invention are provided in the form of a single enantiomer at least 95%, at least 97% and at least 99% free of the corresponding enantiomer.

In a further embodiment the compound of the present invention are in the form of the (+) enantiomer at least 95% free of the corresponding (−) enantiomer.

In a further embodiment the compound of the present invention are in the form of the (+) enantiomer at least 97% free of the corresponding (−) enantiomer.

In a further embodiment the compound of the present invention are in the form of the (+) enantiomer at least 99% free of the corresponding (−) enantiomer.

In a further embodiment, the compounds of the present invention are in the form of the (−) enantiomer at least 95% free of the corresponding (+) enantiomer.

In a further embodiment the compound of the present invention are in the form of the (−) enantiomer at least 97% free of the corresponding (+) enantiomer.

In a further embodiment the compound of the present invention are in the form of the (−) enantiomer at least 99% free of the corresponding (+) enantiomer.

There is also provided pharmaceutically acceptable salts of the compounds of the present invention. By the term pharmaceutically acceptable salts of compounds are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts.

Salts derived from amino acids are also included (e.g. L-arginine, L-Lysine).

Salts derived from appropriate bases include alkali metals (e.g. calcium, sodium, lithium, potassium), alkaline earth metals (e.g. magnesium), ammonium, NR₄_{⁺} (where R is C₁₋₄ alkyl) salts, choline and tromethamine.

A reference hereinafter to a compound according to the invention includes that compound and its pharmaceutically acceptable salts.

In one embodiment of the invention, the pharmaceutically acceptable salt is a sodium salt.

In one embodiment of the invention, the pharmaceutically acceptable salt is a lithium salt.

In one embodiment of the invention, the pharmaceutically acceptable salt is a potassium salt.

It will be appreciated by those skilled in the art that the compounds in accordance with the present invention can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

It will further be appreciated by those skilled in the art that the compounds in accordance with the present invention can exist in different solvate forms, for example hydrates. Solvates of the compounds of the invention may also form when solvent molecules are incorporated into the crystalline lattice structure of the compound molecule during the crystallization process.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

The term “alkyl” represents a linear or branched hydrocarbon moiety. The terms “alkenyl” and “alkynyl” represent a linear, branched or cyclic hydrocarbon moiety which has one or more double bonds or triple bonds in the chain. Examples of alkyl, alkenyl, and alkynyl groups include but are not limited to methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl, butenyl, isobutenyl, hexenyl, butadienyl, pentenyl, pentadienyl, hexenyl, hexadienyl, hexatrienyl, heptenyl, heptadienyl, heptatrienyl, octenyl, octadienyl, octatrienyl, octatetraenyl, propynyl, butynyl, pentynyl, and hexynyl. Where indicated the “alkyl,” “alkenyl,” and “alkynyl” can be optionally substituted such as in the case of haloalkyls in which one or more hydrogen atom is replaced by a halogen, e.g., an alkylhalide. Examples of haloalkyls include but are not limited to trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, dichlorofluoroethyl. Aside from halogens, where indicated, the alkyl, alkenyl or alkynyl groups can also be optionally substituted by, for example, oxo, —NR_{d}Rₑ, —CONR_{d}Rₑ, ═NO—Rₑ, NR_{d}CORₑ, carboxy, —C(═NR_{d})NRₑR_{f}, azido, cyano, C₁₋₆ alkyloxy, C₂₋₆ alkenyloxy, C₂₋₆ alkynyloxy, —N(R_{d})C(═NRₑ)—NR_{f}R_{g}, hydroxyl, nitro, nitroso, —N(Rₕ)CONRᵢRⱼ, S(O)₀₋₂Rₐ, C(O)Rₐ, C(O)ORₐ, NRₐC(O)R_{b}, SO₂NRₐR_{b}, NRₐSO₂R_{b}, NRₐSO₂NR_{b}R_{c}, CRₐN═OR_{b}, and/or NRₐCOOR_{b}, wherein Rₐ-Rⱼ are each independently H, C₁₋₄ alkyl, C₂₋₄ alkenyl or C₂₋₄ alkynyl.

The terms “cycloalkyl”, and “cycloalkenyl” represent a cyclic hydrocarbon alkyl or alkenyl, respectively, and are meant to include monocyclic (e.g., cyclopropyl, cyclobutyl, cyclohexenyl, cyclohexadienyl and cyclohexyl), spiro (e.g., spiro[2.3]hexanyl), fused (e.g., bicyclo[4.4.0]decanyl), and bridged (e.g., bicyclo[2.2.1]heptanyl)hydrocarbon moieties. Where indicated the “cycloalkyl”, and “cycloalkenyl” can be optionally substituted by, for example, oxo, —NR_{d}Rₑ, —CONR_{d}Rₑ, ═NO—Rₑ, NR_{d}CORₑ, carboxy, —C(═NR_{d})NRₑR_{f}, azido, cyano, C₁₋₆ alkyl, C₁₋₆ alkyloxy, C₂₋₆ alkenyloxy, C₂₋₆ alkynyloxy, —N(R_{d})C(═NRₑ)—NR_{f}R_{g}, hydroxyl, nitro, nitroso, —N(Rₕ)CONRᵢRⱼ, S(O)₀₋₂Rₐ, C(O)Rₐ, C(O)ORₐ, NRₐC(O)R_{b}, SO₂NRₐR_{b}, NRₐSO₂R_{b}, NRₐSO₂NR_{b}R_{c}, CRₐN═OR_{b}, and/or NRₐCOOR_{b}, wherein Rₐ-Rⱼ are each independently H, C₁₋₄ alkyl, C₂₋₄ alkenyl or C₂₋₄ alkynyl.

The terms “alkoxy,” “alkenyloxy,” and “alkynyloxy” represent an alkyl, alkenyl or alkynyl moiety, respectively, which is covalently bonded to the adjacent atom through an oxygen atom. Like the alkyl, alkenyl and alkynyl groups, where indicated the alkoxy, alkenyloxy and alkynyloxy groups can also be optionally substituted. Examples include but are not limited to methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy, trifluoromethoxy and neohexyloxy. The alkoxy, alkenyloxy, and alkynyloxy groups can be optionally substituted by, for example, halogens, oxo, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, C₁₋₆ alkyl, C₂₋₆ alkenyl, C₂₋₆ alkynyl, —N(Rₕ)CONRᵢRⱼ, S(O)₀₋₂Rₐ, C(O)Rₐ, C(O)ORₐ, ═NO—Rₑ, NRₐC(O)R_{b}, SO₂NRₐR_{b}, NRₐSO₂R_{b}, NRₐSO₂NR_{b}R_{c}, CRₐN═OR_{b}, and/or NRₐCOOR_{b}, wherein Rₐ—Rⱼ are each independently H, C₁₋₄ alkyl, C₂₋₄ alkenyl or C₂₋₄ alkynyl.

The term “aryl” represents a carbocyclic moiety containing at least one benzenoid-type ring (i.e., may be monocyclic or polycyclic), and which where indicated may be optionally substituted with one or more substituents. Examples include but are not limited to phenyl, tolyl, dimethylphenyl, aminophenyl, anilinyl, naphthyl, anthryl, phenanthryl, and biphenyl. The aryl groups can be optionally substituted by, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, S(O)₀₋₂Rₐ, optionally substituted 5-12 member heteroaryl, optionally substituted 6-18 member heteroaralkyl, optionally substituted 3-12 member heterocycle, optionally substituted 4-18 member heterocycle-alkyl, C(O)Rₐ, C(O)ORₐ, NRₐC(O)R_{b}, SO₂NRₐR_{b}, NRₐSO₂R_{b}, NRₐSO₂NR_{b}R_{c}, CRₐN═OR_{b}, and/or NRₐCOOR_{b}, wherein Rₐ-Rⱼ are each independently H, C₁₋₄ alkyl, C₂₋₄ alkenyl or C₂₋₄ alkynyl.

The term “aralkyl” represents an aryl group attached to the adjacent atom by an alkyl, alkenyl or alkynyl. Like the aryl groups, where indicated the aralkyl groups can also be optionally substituted. Examples include but are not limited to benzyl, benzhydryl, trityl, phenethyl, 3-phenylpropyl, 2-phenylpropyl, 4-phenylbutyl and naphthylmethyl. Where indicated, the aralkyl groups can be optionally substituted by, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, S(O)₀₋₂Rₐ, optionally substituted 5-12 member heteroaryl, optionally substituted 6-18 member heteroaralkyl, optionally substituted 3-12 member heterocycle, optionally substituted 4-18 member heterocycle-alkyl, C(O)Rₐ, C(O)ORₐ, NRₐC(O)R_{b}, SO₂NRₐR_{b}, NRₐSO₂R_{b}, NRₐSO₂NR_{b}R_{c}, CRₐN═OR_{b}, and/or NRₐCOOR_{b}, wherein Rₐ-Rⱼ are each independently H, C₁₋₄ alkyl, C₂₋₄ alkenyl or C₂₋₄ alkynyl.

The term “heterocycle” represents an optionally substituted, non aromatic, saturated or partially saturated wherein said cyclic moiety is interrupted by at least one heteroatom selected from oxygen (O), sulfur (S) or nitrogen (N). Heterocycles may be monocyclic or polycyclic rings. Examples include but are not limited to azetidinyl, dioxolanyl, morpholinyl, morpholino, oxetanyl, piperazinyl, piperidyl, piperidinyl, piperidino, cyclopentapyrazolyl, cyclopentaoxazinyl, cyclopentafuranyl, tetrahydrofuranyl, tetrahydropyranyl, tetrahydrothiopyranyl, oxazolinyl, oxazinyl, pyranyl, aziridinyl, azepinyl, dioxazepinyl, diazepinyl, oxyranyl, pyrrolidinyl, and thiopyranyl. Where indicated, the heterocyclic groups can be optionally substituted by, for example, halogens, oxo, —NR_{d}Rₑ, —CONR_{d}Rₑ, ═NO—Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₇₋₁₂ aralkyl, C₆₋₁₂ aryl, C₁₋₆ alkyloxy, C₂₋₆ alkenyloxy, C₂₋₆ alkynyloxy, S(O)₀₋₂Rₐ, C₆₋₁₀ aryl, C₆₋₁₀ aryloxy, C₇₋₁₀ arylalkyl, C₆₋₁₀ aryl-C₁₋₁₀ alkyloxy, C(O)Rₐ, C(O)ORₐ, NRₐC(O)R_{b}, SO₂NRₐR_{b}, NRₐSO₂R_{b}, NRₐSO₂NR_{b}R_{c}, CRₐN═OR_{b}, and/or NRₐCOOR_{b}, wherein Rₐ-Rⱼ are each independently H, C₁₋₄ alkyl, C₂₋₄ alkenyl or C₂₋₄ alkynyl.

The term “heterocycle-alkyl” represents an optionally substituted heterocycle group attached to the adjacent atom by an alkyl, alkenyl or alkynyl group. It is understood that in a 5-18 member heterocycle-alkyl moiety, the 5-18 member represent the atoms that are present in both the heterocycle moiety and the alkyl, alkenyl or alkynyl group. For example, the following groups are encompassed by a 7 member heterocycle-alkyl (* represents the attachment point):

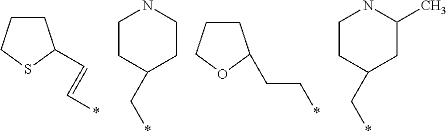

Where indicated the heterocycle-alkyl groups can be optionally substituted by, for example, halogens, oxo, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, S(O)₀₋₂Rₐ, C₆₋₁₀ aryl, C₆₋₁₀ aryloxy, C₇₋₁₀ arylalkyl, C₆₋₁₀ aryl-C₁₋₁₀ alkyloxy, C(O)Rₐ, C(O)ORₐ, NRₐC(O)R_{b}, ═NO—Rₑ, SO₂NRₐR_{b}, NRₐSO₂R_{b}, NRₐSO₂NR_{b}R_{c}, CRₐN═OR_{b}, and/or NRₐCOOR_{b}, wherein Rₐ-Rⱼ are each independently H, C₁₋₄ alkyl, C₂₋₄ alkenyl or C₂₋₄ alkynyl.

The term “heteroaryl” represents an optionally substituted aromatic cyclic moiety wherein said cyclic moiety is interrupted by at least one heteroatom selected from oxygen (O), sulfur (S) or nitrogen (N). Heteroaryls may be monocyclic or polycyclic rings. Examples include but are not limited to azetyl, dithiadiazinyl, dithiazolyl, furanyl, isooxazolyl, isothiazolyl, imidazolyl, oxadiazolyl, oxazolyl, pyrazinyl, pyridazinyl, pyrimidinyl, pyridyl, pyrazolyl, pyrrolyl, thiatriazolyl, tetrazolyl, thiadiazolyl, triazolyl, thiazolyl, thienyl, tetrazinyl, thiadiazinyl, triazinyl, thiazinyl, furoisoxazolyl, imidazothiazolyl, thienoisothiazolyl, thienothiazolyl, imidazopyrazolyl, to pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, isoindolinyl, furopyrimidinyl, furopyridyl, benzofuranyl, isobenzofuranyl, thienopyrimidinyl, thienopyridyl, benzothienyl, benzoxazinyl, benzothiazinyl, quinazolinyl, naphthyridinyl, quinolinyl, isoquinolinyl, benzopyranyl, pyridopyridazinyl and pyridopyrimidinyl. Where indicated the heteroaryl groups can be optionally substituted by, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, S(O)₀₋₂Rₐ, C₆₋₁₀ aryl, C₆₋₁₀ aryloxy, C₇₋₁₀ arylalkyl, C₆₋₁₀ aryl-C₁₋₁₀ alkyloxy, C(O)Rₐ, C(O)ORₐ, NRₐC(O)R_{b}, SO₂NRₐR_{b}, NRₐSO₂R_{b}, NRₐSO₂NR_{b}R_{c}, CRₐN═OR_{b}, and/or NRₐCOOR_{b}, wherein Rₐ-Rⱼ are each independently H, C₁₋₄ alkyl, C₂₋₄ alkenyl or C₂₋₄ alkynyl.

The term “heteroaralkyl” represents an optionally substituted heteroaryl group attached to the adjacent atom by an alkyl, alkenyl or alkynyl group. Where indicated the heteroaralkyl groups can be optionally substituted by, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, S(O)₀₋₂Rₐ, C₆₋₁₀ aryl, C₆₋₁₀ aryloxy, C₇₋₁₀ arylalkyl, C₆₋₁₀ aryl-C₁₋₁₀ alkyloxy, C(O)Rₐ, C(O)ORₐ, NRₐC(O)R_{b}, SO₂NRₐR_{b}, NRₐSO₂R_{b}, NRₐSO₂NR_{b}R_{c}, CRₐN═OR_{b}, and/or NRₐCOOR_{b}, wherein Rₐ-Rⱼ are each independently H, C₁₋₄ alkyl, C₂₋₄ alkenyl or C₂₋₄ alkynyl. It is understood that in a 6-18 member heteroaralkyl moiety, the 6-18 member represents the atoms that are present in both the heterocycle moiety and the alkyl, alkenyl or alkynyl groups. For example, the following groups are encompassed by a 7 member heteroaralkyl (* represents the attachment point):

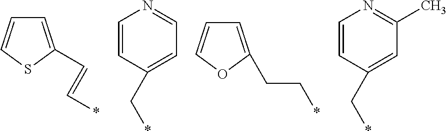

“Halogen atom” is specifically a fluorine atom, chlorine atom, bromine atom or iodine atom.

The term “oxo” represents ═O.

The term “amidino” represents —C(═NR_{w}) NRₓR_{y} wherein R_{w}, Rₓ and R_{y} are each independently selected from H, C₁₋₁₀ alkyl, C₂₋₁₀ alkenyl, C₂₋₁₀ alkynyl, C₆₋₁₂ aryl and C₇₋₁₂ aralkyl, or Rₓ and R_{y} are taken together with the nitrogen to which they are attached to form an optionally substituted 4 to 10 member heterocycle or an optionally substituted 5-12 member heteroaryl.

The term “guanidino” represents —N(R_{w})C(═NRₓ)NR_{y}R_{z} wherein R_{w}, Rₓ, R_{y} and R_{z} are each independently selected from H, C₁₋₁₀ alkyl, C₂₋₁₀ alkenyl, C₂₋₁₀ alkynyl, C₆₋₁₂ aryl and C₇₋₁₂ aralkyl, or R_{y} and R_{z} are taken together with the nitrogen to which they are attached to form an optionally substituted 4 to 10 member heterocycle or an optionally substituted 5-12 member heteroaryl.

The term “amido” represents —CONRₓR_{y} and —NRₓCOR_{y}, wherein Rₓ and R_{y} are each independently selected from H, C₁₋₁₀ alkyl, C₂₋₁₀ alkenyl, C₂₋₁₀ alkynyl, C₆₋₁₂ aryl and C₇₋₁₂ aralkyl, or Rₓ and R_{y} are taken together with the nitrogen to which they are attached (or the nitrogen atom and CO group in the case of —NRₓCOR_{y}) to form an optionally substituted 4 to 10 member heterocycle or an optionally substituted 5-12 member heteroaryl.

The term “amino” represents a derivative of ammonia obtained by substituting one or more hydrogen atom and includes —NRₓR_{y}, wherein Rₓ and R_{y} are each independently selected from H, C₁₋₁₀ alkyl, C₂₋₁₀ alkenyl, C₂₋₁₀ alkynyl, C₆₋₁₂ aryl and C₇₋₁₂ aralkyl, or Rₓ and R_{y} are taken together with the nitrogen to which they are attached to form an optionally substituted 4 to 10 member heterocycle or an optionally substituted 5-12 member heteroaryl.

The term “sulfonamido” represents SO₂NRₓR_{y}, and —NRₓSO₂R_{y}, wherein Rₓ and R_{y} are each independently selected from H, C₁₋₁₀ alkyl, C₂₋₁₀ alkenyl, C₂₋₁₀ alkynyl, C₆₋₁₂ aryl and C₇₋₁₂ aralkyl, or Rₓ and R_{y} are taken together with the nitrogen to which they are attached to form an optionally substituted 4 to 10 member heterocycle or an optionally substituted 5-12 member heteroaryl.

When there is a sulfur atom present, the sulfur atom can be at different oxidation levels, i.e., S, SO, or SO₂. All such oxidation levels are within the scope of the present invention.

The term “independently” means that a substituent can be the same or a different definition for each item.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

The compound is conveniently administered in unit dosage form; for example containing 10 to 1500 mg, conveniently 20 to 1000 mg, most conveniently 50 to 700 mg of active ingredient per unit dosage form.

Ideally the active ingredient should be administered to achieve peak plasma concentrations of the active compound of from about 1 to about 75 μM, about 2 to 50 μM, about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

When the compounds of the present invention or a pharmaceutically acceptable salts thereof is used in combination with a second therapeutic agent active against the same virus the dose of each compound may be either the same as or differ from that when the compound is used alone. Appropriate doses will be readily appreciated by those skilled in the art.

While it is possible that, for use in therapy, a compound of the invention may be administered as the raw chemical it is preferable to present the active ingredient as a pharmaceutical composition. The invention thus further provides a pharmaceutical composition comprising compounds of the present invention or a pharmaceutically acceptable derivative thereof together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient thereof.

Pharmaceutical compositions include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

For topical administration to the epidermis, the compounds according to the invention may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Compositions suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid are for example presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Compositions suitable for vaginal administration may be presented as pessaries, tampons, creams, gels, pastes, foams or sprays containing in addition to the active ingredient such carriers as are known in the art to be appropriate.

For intra-nasal administration the compounds of the invention may be used as a liquid spray or dispersible powder or in the form of drops. Drops may be formulated with an aqueous or non-aqueous base also comprising one more dispersing agents, solubilizing agents or suspending agents. Liquid sprays are conveniently delivered from pressurized packs.

For administration by inhalation the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

When desired the above described formulations adapted to give sustained release of the active ingredient may be employed.

The following general schemes and examples are provided to illustrate various embodiments of the present invention and shall not be considered as limiting in scope. It will be appreciated by those of skill in the art that other compounds of the present invention can be obtained by substituting the generically or specifically described reactants and/or operating conditions used in the following examples. Synthesis methods to obtain thiophene compounds are also described in patent applications WO02/100851, U.S. Pat. No. 6,881,741, WO2004/052885, US 2005-0009804, WO2004/052879, US 2004-0192707, WO2006/119646, US 2006-0276533, WO 2006/072347, WO 2006/072348 and WO2008/058393 the disclosures of which are hereby incorporated by reference.

In the foregoing and in the following examples, all temperatures are set forth uncorrected in degrees Celsius; and, unless otherwise indicated, all parts and percentages are by weight.

The following abbreviations may be used as follows:
AcOH Acetic acid
Ac Acetate
Cpd Compound
DMF N,N-dimethylformamide
Eq equivalent
THF tetrahydrofuran

Purifications by HPLC are all performed using reverse phase C18 column packed with 5 μm particles. Column diameter is 19 mm and length is 100 mm. Eluent is an appropriate gradient of acetonitrile and water with a 3 mM HCl concentration.

### EXEMPLIFICATION

The compounds according to the invention described herein can be prepared by any suitable method known in the art, for example, U.S. Pat. No. 6,881,741, US 2005/0009804, US 2006/0276533, WO 2002/100851, and WO 08/58393. Preparation details of some exemplary compounds are described below. Generally, the compounds of the invention can be prepared as shown in those syntheses optionally with any desired appropriate modification.

### General Analytical Methods and Methodology for Synthesis and Characterization of Compounds

As used herein the term RT (min) refers to the LCMS retention time, in minutes, associated with the compound. NMR and Mass Spectroscopy data of certain specific compounds are summarized in Table 2.

### Example 1

### General Scheme for Synthesis of Thiophene Analogs

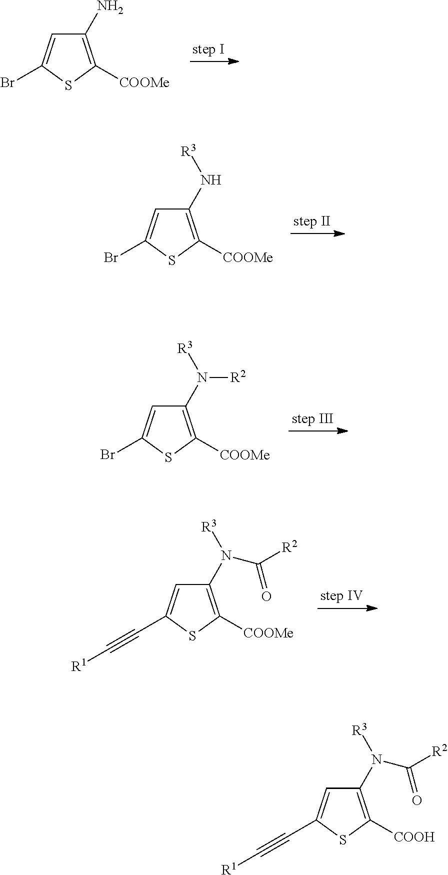

### Step I

To a stirred solution of 3-amino-5-bromo-thiophene-2-carboxylic acid methyl ester in 1,2-dichloroethane (0.8 M), ketone of R³ or its derivatives (4 eq), AcOH (4 eq) and NaBH(OAc)₃ (4 eq) are added sequentially. The reaction mixture is stirred at rt for overnight. It is then diluted with chloroform and water. The organic layer is separated, dried with sodium sulfate (Na₂SO₄) and filtered. The solvents are removed under rotary evaporator and the residue is purified by column chromatography to obtain the alkylated amine.

### Step II

To a solution of alkylated amine in dichloromethane (0.15 M), unsubstituted or substituted benzoyl chloride (2 eq), N-chlorosuccinamide (1 eq) and triphenylphosphine (2 eq) are added at room temperature. The reaction mixture is refluxed for 16 h. It is then diluted with chloroform and aqueous NaHCO₃ solution. The organic layer is separated, dried (Na₂SO₄), filtered and concentrated. The residue is purified by column chromatography to obtain the acylated compound.

### Step III

A mixture of acylated compound, tris(dibenzylideneacetone)dipalladium(0) (0.04 eq) and copper(I) iodide (0.04 eq) in anhydrous DMF is treated with triethylamine (2.5 eq). R¹-butyne is added and the resulting mixture is stirred at 60° C. for 24 h. The reaction mixture is allowed to cool down to room temperature. It is diluted with ethyl acetate, washed with three portions of water, dried over sodium sulfate and concentrated. The residue is purified by silica gel column chromatography to obtain a mixture of the desired alkyne compound and starting material. This mixture is used for next step without any further purification.

### Step IV

To a solution of alkyne compound in THF, methanol, water (ratio 3:2:1) is added lithium hydroxide monohydrate (5-10 eq). The resulting mixture is stirred at room temperature for 16 h. Solvent is removed. A portion of water is added and the mixture is acidified to about pH 3 using a 1N aqueous solution of HCl. Then, it is extracted with ethyl acetate. The organic portion is washed water, dried over sodium sulfate and concentrated. The crude is purified by HPLC preparative to afford the final product.

### Example 2

### Exemplary Syntheses of Certain Compounds of the Invention

### Preparation of 3-[(2,4-dichlorobenzoyl)isopropylamino]-5-(3,3-dimethylbut-1-ynyl)thiophene-2-carboxylic acid (Compound 1)

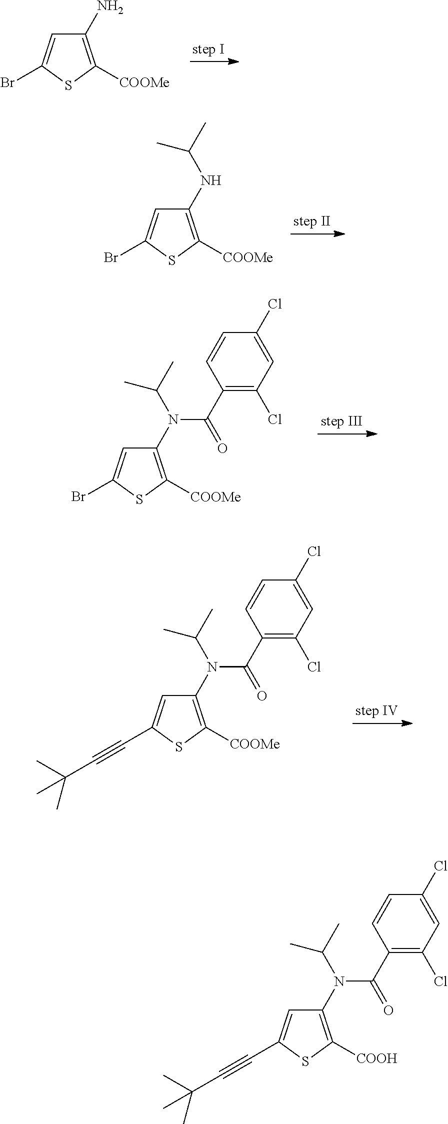 [SMILES: COC(=O)c1sc(Br)cc1N ; COC(=O)c1sc(Br)cc1NC(C)C ; COC(=O)c1sc(Br)cc1N(C(=O)c1ccc(Cl)cc1Cl)C(C)C ; COC(=O)c1sc(C#CC(C)(C)C)cc1N(C(=O)c1ccc(Cl)cc1Cl)C(C)C ; CC(C)N(C(=O)c1ccc(Cl)cc1Cl)c1cc(C#CC(C)(C)C)sc1C(=O)O]

### Step I

To a stirred solution of 3-amino-5-bromo-thiophene-2-carboxylic acid methyl ester (4.0 g, 16.95 mmol) in 1,2-dichloroethane (20 mL), 2-methoxypropene (6.5 mL, 67.79 mmol), AcOH (3.8 mL, 67.79 mmol) and NaBH(OAc)₃ (7.2 g, 67.79 mmol) were added sequentially. The reaction mixture was stirred at room temperature for overnight. It was then diluted with chloroform and water. The organic layer was separated, dried (Na₂SO₄) and filtered. The solvents were removed under rotary evaporator and the residue was purified by column chromatography using ethyl acetate and hexane (0:1 to 1:1) to obtain 5-bromo-3-isopropylamino-thiophene-2-carboxylic acid methyl ester (4.0 g).

### Step II

To a solution of 5-bromo-3-isopropylamino-thiophene-2-carboxylic acid methyl ester (220 mg, 0.79 mmol) in dichloromethane (5 mL), 2,4-dichloro-benzoyl chloride (331 mg, 1.58 mmol), N-chlorosuccinamide (105 mg, 0.79 mmol) and triphenylphosphine (414 mg, 1.58 mmol) were added at room temperature. The reaction mixture was refluxed for 16 h. It was then diluted with chloroform and aqueous NaHCO₃ solution. The organic layer was separated, dried (Na₂SO₄), filtered and concentrated. The residue was purified by column chromatography using ethyl acetate and hexane (0:1 to 1:1) to obtain 5-bromo-3-[(2,4-dichloro-benzoyl)-isopropyl-amino]-thiophene-2-carboxylic acid methyl ester (259 mg).

### Step III

A mixture of 5-bromo-3-[(2,4-dichlorobenzoyl)-isopropylamino]-thiophene-2-carboxylic acid methyl ester (503 mg, 1.115 mmol), tris(dibenzylideneacetone)dipalladium(0) (41 mg, 0.045 mmol) and copper(I) iodide (8.5 mg, 0.045 mmol) in 4.9 mL of anhydrous DMF was treated with triethylamine (390 μL, 2.79 mmol). 3,3-Dimethyl-1-butyne was added and the resulting mixture was stirred at 60° C. for 24 h. The reaction mixture was allowed to cool down to room temperature. It was diluted with 100 mL of ethyl acetate, washed with three portions of 75 ml of water, dried over sodium sulfate and concentrated. The residue was purified by silica gel column chromatography using a gradient from 0% to 50% ethyl acetate:hexanes as eluent. The white solid (401 mg) contained a 70:30 ratio of the 3-[(2,4-dichlorobenzoyl)isopropylamino]-5-(3,3-dimethylbut-1-ynyl)thiophene-2-carboxylic acid methyl ester and the starting 5-bromo-3-[(2,4-dichlorobenzoyl)isopropylamino]-thiophene-2-carboxylic. This mixture was used for next step without any further purification.

### Step IV

To a solution of 3-[(2,4-dichlorobenzoyl)-isopropylamino]-5-(3,3-dimethylbut-1-ynyl)thiophene-2-carboxylic acid methyl ester (391 mg, 70% of purity) in 14 mL of a 3:2:1 ratio of THF, methanol, water was added lithium hydroxide monohydrate (145 mg, 3.46 mmol). The resulting mixture was stirred at room temperature for 16 h. Solvent was removed. A portion of 30 mL of water was added. It was acidified to about pH 3 using a 1N aqueous solution of HCl. Then, it was extracted with 40 mL of ethyl acetate. The organic portion was washed with 20 mL of water, dried over sodium sulfate and concentrated. The crude was purified by HPLC preparative using a Phenomenex AXIA Gemini 5u C18 110A 100 mm×30 mm column with a gradient of 39% to 69% acetonitrile:3 mM aqueous HCl in 90 min and a flow rate of 12 mL/min. It afforded 3-[(2,4-dichlorobenzoyl)isopropylamino]-5-(3,3-dimethylbut-1-ynyl)thiophene-2-carbo-xylic acid (151 mg over two steps) as a white solid. M+H⁺=440.0

### Preparation of Compound 18: 3-[(2,4-Dimethyl-benzoyl)-(trans-4-hydroxy-cyclohexyl)-amino]-5-(3,3-dimethyl-but-1-ynyl)-thiophene-2-carboxylic acid

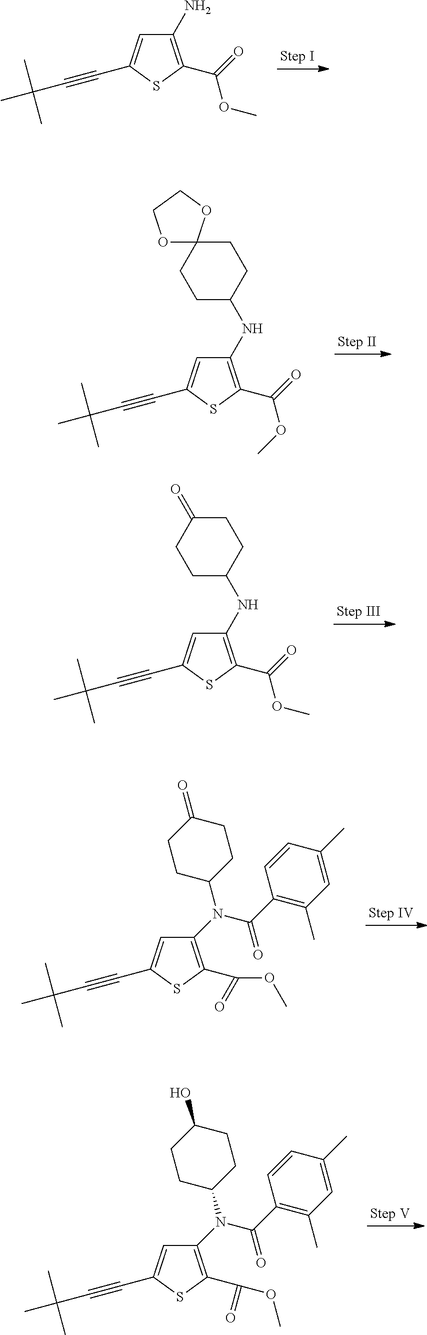

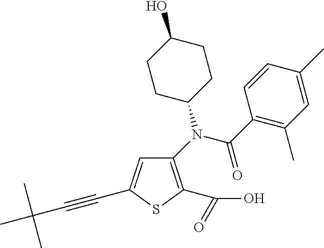 [SMILES: Cc1ccc(C(=O)N(c2cc(C#CC(C)(C)C)sc2C(=O)O)[C@H]2CC[C@H](O)CC2)c(C)c1]

### Step I

To a solution of 3-amino-5-(3,3-dimethyl-but-1-ynyl)-thiophene-2-carboxylic acid methyl ester (3.18 g, 13.1 mmol) in toluene (16 mL) was sequentially added 1,4-cyclohexanedione monoethylene ketal (4.09 g, 26.2 mmol), acetic acid (750 μL, 0.0131 mmol) and sodium triacetoxyborohydride (5.55 g, 26.2 mmol) under nitrogen atmosphere. The reaction mixture was stirred at RT overnight, filtered and washed with toluene (10 mL). The organic layer was washed with saturated sodium bicarbonate solution (1×10 mL), and EtOAc (1×20 mL) and concentrated to dryness. The residue was purified by flash column chromatography on silica gel (100% DCM) to give 5-(3,3-dimethyl-but-1-ynyl)-3-(1,4-dioxa-spiro[4.5]dec-8-ylamino)-thiophene-2-carboxylic acid methyl ester (4.1 g, 83%).

### Step II

To a solution of 5-(3,3-dimethyl-but-1-ynyl)-3-(1,4-dioxa-spiro[4.5]dec-8-ylamino)-thiophene-2-carboxylic acid methyl (4.0 g, 10.16 mmol) in THF (20 mL) was added aqueous HCl (20 mL, 3.6 N) under nitrogen atmosphere. The reaction mixture was stirred overnight at 40° C. Additional THF (30 mL) and HCl (5 mL, 12 N) were added and the mixture is stirred overnight at 40° C., cooled to RT and diluted with THF (10 mL). The organic layer was diluted with water (1×20 mL) and THF was evaporated to form a precipitate in H₂O. The precipitate was filtered, washed with H₂O and co-evaporated with toluene to give 5-(3,3-dimethyl-but-1-ynyl)-3-(4-oxo-cyclohexylamino)-thiophene-2-carboxylic acid methyl ester (2.75 g, 73%).

### Step III

To a solution of 5-(3,3-dimethyl-but-1-ynyl)-3-(4-oxo-cyclohexylamino)-thiophene-2-carboxylic acid methyl ester (200 mg, 0.539 mmol) in toluene (5 mL) was sequentially added pyridine (85 mL, 1.08 mmol) and 2,4-dimethylbenzoyl chloride (182 mL, 1.08 mmol) under nitrogen atmosphere. The reaction mixture was heated at 110° C. overnight in a sealed tube, cooled to RT, and diluted with EtOAc (10 mL). The reaction mixture was washed with saturated sodium bicarbonate solution (1×5 mL), dried over Na₂SO₄, filtered, and concentrated to dryness. The residue was purified by flash column chromatography on silica gel (0 to 50% EtOAc in Hexanes) to give 3-[(2,4-dimethyl-benzoyl)-(4-oxo-cyclohexyl)-amino]-5-(3,3-dimethyl-but-1-ynyl)-thiophene-2-carboxylic acid methyl ester (200 mg, 80%).

### Step IV

Sodium borohydride (16 mg, 0.43 mmol) was added to THF (2 mL) and H₂O (40 μL) at −20° C. To this mixture was added a solution of 3-[(2,4-dimethyl-benzoyl)-(4-oxo-cyclohexyl)-amino]-5-(3,3-dimethyl-but-1-ynyl)-thiophene-2-carboxylic acid methyl ester (200 mg, 0.43 mmol) in THF (4 mL) under nitrogen atmosphere. The reaction mixture was stirred at −20° C. for 30 minutes and aqueous HCl 1N (2 mL) was added. The reaction mixture was extracted with EtOAc (2×5 mL), and the organic layer was dried over Na₂SO₄, filtered, and concentrated to dryness. The residue was purified by flash column chromatography on silica gel (0 to 50% EtOAc in Hexanes) to give 3-[(2,4-dimethyl-benzoyl)-(trans-4-hydroxy-cyclohexyl)-amino]-5-(3,3-dimethyl-but-1-ynyl)-thiophene-2-carboxylic acid methyl ester (123 mg, 61%).

### Step V

To a solution of 3-[(2,4-dimethyl-benzoyl)-(trans-4-hydroxy-cyclohexyl)-amino]-5-(3,3-dimethyl-but-1-ynyl)-thiophene-2-carboxylic acid methyl ester (80 mg, 0.171 mmol) in a 3:2:1 mixture of THF: methanol: H₂O (2 mL) was added lithium hydroxide monohydride (20 mg, 0.856 mmol) under nitrogen atmosphere. The reaction mixture was stirred overnight and acidified to pH 3-4 with aqueous HCl 1N. The reaction mixture was extracted with EtOAc (2×5 mL), and the combined organic layers are dried over Na₂SO₄, filtered, and concentrated to dryness. The residue was purified by flash column chromatography on silica gel (0 to 10% methanol in DCM) to give 3-[(2,4-dimethyl-benzoyl)-(trans-4-hydroxy-cyclohexyl)-amino]-5-(3,3-dimethyl-but-1-ynyl)-thiophene-2-carboxylic acid (35 g, 45%).

¹H NMR (400 MHz, DMSO-d₆): δ 7.15-7.01 (m, 2H), 6.86 (s, 1H), 6.72 (d, J=7.7 Hz, 1H), 4.61-4.33 (m, 2H), 3.44-3.21 (m, 2H), 2.18 (d, J=17.2 Hz, 6H), 2.03-1.96 (d, J=11.9 Hz, 1H), 1.90-1.75 (m, 3H), 1.45-1.28 (m, 3H), 1.25 (s, 9H), 0.99-0.85 (m, 1H).

LC/MS: m/z=454.13 (M+H⁺).

### Preparation of Compounds 2, 17, 51 and 22

The following compounds were prepared using essentially the same procedure described above Compound 18:

### Compound 2: 3-[(2,4-Dichloro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 13.71 (s, 1H), 7.54 (d, 1H), 7.35 (dd, 1H), 7.25 (d, 1H), 7.21 (s, 1H), 4.58 (s, 1H), 4.44-4.34 (m, 1H), 3.30-3.23 (m, 1H), 2.05-1.99 (m, 1H), 1.93-1.69 (m, 4H), 1.53-1.41 (m, 2H), 1.27 (s, 9H), 1.02-0.90 (m, 2H).

LC/MS: m/z=494.03 (M+H⁺).

### Compound 17: 3-[(4-Chloro-2-fluoro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 7.51 (m, 1H), 7.26 (m, 1H), 7.08 (m, 1H), 6.84 (s, 1H), 4.53 (bs, 1H), 4.34 (m, 1H), 3.25 (m, 1H), 1.95-1.84 (m, 3H), 1.82-1.73 (m, 1H), 1.44-1.12 (m, 12H), 0.99-0.84 (m, 1H).

LC/MS: m/z=478.04 (M+H⁺)

### Compound 51: 5-(3,3-Dimethyl-but-1-ynyl)-3-[(4-fluoro-benzoyl)-(trans-4-hydroxy-cyclohexyl)-amino]-thiophene-2-carboxylic acid

LC/MS: m/z=444.09 (M+H⁺)

### Compound 22: 5-(3,3-Dimethyl-but-1-ynyl)-3-[(trans-4-hydroxy-cyclohexyl)-(4-trifluoromethyl-benzoyl)-amino]-thiophene-2-carboxylic acid)

LC/MS: m/z=494.08 (M+H⁺)

### Preparation of Compound 53: 3-[(2-Chloro-4-methyl-benzoyl)-(trans-4-hydroxy-cyclohexyl)-amino]-5-(3,3-dimethyl-but-1-ynyl)-thiophene-2-carboxylic acid

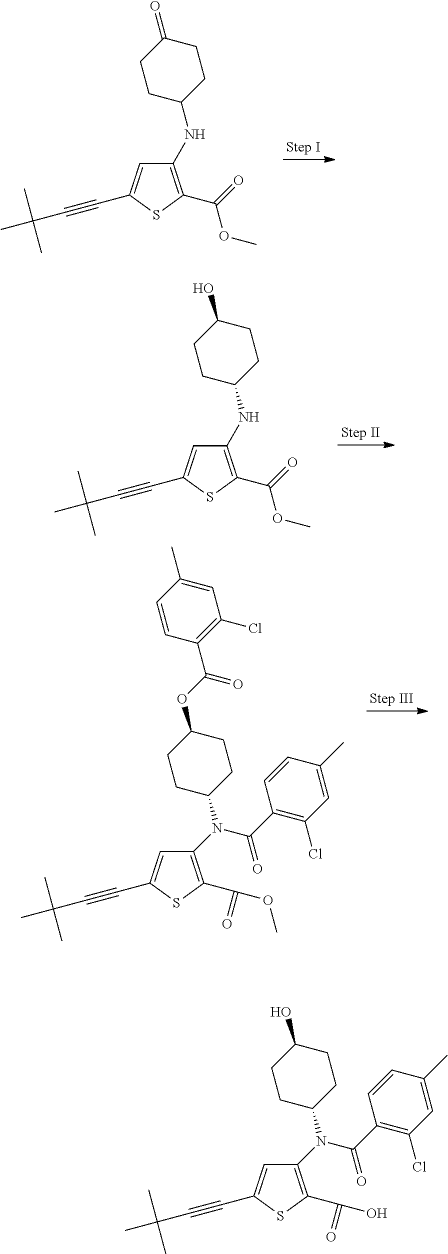

### Step I

Sodium borohydride (170 mg, 4.49 mmol) was added to a mixture of THF (15 mL) and H₂O (300 μL) at −15° C. To this mixture was added a solution of 5-(3,3-dimethyl-but-1-ynyl)-3-(4-oxo-cyclohexylamino)-thiophene-2-carboxylic acid methyl ester (1.58 g, 4.49 mmol) in THF (15 mL) under nitrogen atmosphere. The reaction mixture was stirred at −15° C. for 45 minutes, warmed to RT and aqueous HCl (2 mL, 1 N) was added. The reaction mixture was extracted by EtOAc (2×20 mL), and the organic layer was dried over Na₂SO₄, filtered, and concentrated to dryness. The residue was purified by flash column chromatography on silica gel (0 to 50% EtOAc in Hexanes) to give 5-(3,3-dimethyl-but-1-ynyl)-3-(trans-4-hydroxy-cyclohexylamino)-thiophene-2-carboxylic acid methyl ester (1.32 g, 88%).

### Step II

To a solution of 2-chloro-4-methylbenzoyl chloride (280 mg, 1.48 mmol) in toluene (2 mL) was sequentially added 5-(3,3-dimethyl-but-1-ynyl)-3-(trans-4-hydroxy-cyclohexylamino)-thiophene-2-carboxylic acid methyl ester (125 g, 0.37 mmol) and pyridine (140 μL, 1.74 mmol) under nitrogen atmosphere. The reaction mixture was stirred overnight at 100° C., cooled to RT, and concentrated to dryness. The residue was purified by flash column chromatography on silica gel (0 to 50% EtOAc in Hexanes) to give 3-{(2-chloro-4-methyl-benzoyl)-[trans-4-(2-chloro-4-methyl-benzoyloxy)-cyclohexyl]-amino}-5-(3,3-dimethyl-but-1-ynyl)-thiophene-2-carboxylic acid methyl ester (175 mg, 74%).

### Step III

To a solution of 3-{(2-chloro-4-methyl-benzoyl)-[trans-4-(2-chloro-4-methyl-benzoyloxy)-cyclohexyl]-amino}-5-(3,3-dimethyl-but-1-ynyl)-thiophene-2-carboxylic acid methyl ester (175 mg, 0.21 mmol) in a 3:2:1 mixture of THF:methanol:H₂O (2 mL) was added lithium hydroxide (170 mg, 2.7 mmol) under nitrogen atmosphere. The reaction mixture was stirred overnight and acidified to pH 3-4 with aqueous HCl 1 N. The reaction mixture was extracted by EtOAc (2×3 mL), and the combined organic layers are dried over Na₂SO₄, filtered, and concentrated to dryness. The residue was purified by flash column chromatography on silica gel (0 to 10% methanol in DCM) to give 3-[(2-chloro-4-methyl-benzoyl)-(trans-4-hydroxy-cyclohexyl)-amino]-5-(3,3-dimethyl-but-1-ynyl)-thiophene-2-carboxylic acid (35 g, 45%).

¹H NMR (400 MHz, DMSO-d₆): δ 7.14 (d, 3H), 7.00 (d, 1H), 4.56 (bs, 1H), 4.45-4.33 (m, 1H), 3.30 (m, 1H), 2.21 (s, 3H), 2.06-1.98 (m, 1H), 1.93-1.70 (m, 4H), 1.52-1.39 (m, 2H), 1.34-1.28 (m, 2H), 1.26 (s, 9H), 1.24-1.12 (m, 2H), 1.01-0.88 (m, 1H).

LC/MS: m/z=474.07 (M+H⁺).

### Preparation of Compounds 54, 19, 87, 88, 89, 16, 24 and 52

The following compounds were prepared using essentially the same procedure described above for Compound 53:

### Compound 54: 5-(3,3-Dimethyl-but-1-ynyl)-3-[(2-fluoro-4-methyl-benzoyl)-(trans-4-hydroxy-cyclohexyl)-amino]-thiophene-2-carboxylic acid

¹H NMR (400 MHz, DMSO-d₆): δ 13.48 (s, 1H), 7.17 (s, 1H), 7.10 (t, 1H), 6.86 (d, 2H), 4.55 (d, 1H), 4.45-4.32 (m, 1H), 3.31-3.20 (m, 1H), 2.22 (s, 3H), 2.00-1.69 (m, 5H), 1.55-1.39 (m, 2H), 1.28 (s, 9H), 1.07-0.89 (m, 2H).

LC/MS: m/z=458.11 (M+H⁺).

### Compound 19: 5-(3,3-Dimethyl-but-1-ynyl)-3-[(trans-4-hydroxy-cyclohexyl)-(4-methyl-benzoyl)-amino]-thiophene-2-carboxylic acid

LC/MS: m/z=440.13 (M+H⁺).

### Compound 87: 3-[(2-Chloro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 13.66 (s, 1H), 7.36-7.12 (m, 5H), 4.57 (s, 1H), 4.41 (s, 1H), 3.30-3.22 (m, 1H), 2.09-1.70 (m, 5H), 1.54-1.39 (m, 2H), 1.35-1.18 (m, 9H), 1.03-0.89 (m, 1H).

LC/MS: m/z=461.94 (M+H⁺).

### Compound 88: 5-(3,3-Dimethyl-but-1-ynyl)-3-[(trans-4-hydroxy-cyclohexyl)-(2-methyl-benzoyl)-amino]-thiophene-2-carboxylic acid

¹H NMR (400 MHz, DMSO-d₆): δ 13.49 (s, 1H), 7.24 (s, 1H), 7.13-6.92 (m, 4H), 4.56 (d, 1H), 4.48-4.36 (m, 1H), 3.31-3.22 (m, 1H), 2.23 (s, 3H), 2.06-1.72 (m, 5H), 1.54-1.39 (m, 2H), 1.33-1.31 (m, 1H), 1.26 (s, 9H), 1.04-0.90 (m, 2H).

LC/MS: m/z=439.98 (M+H⁺).

### Compound 89: 3-[(2,3-Difluoro-4-methyl-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 13.56 (s, 1H), 7.25 (s, 1H), 7.03-6.83 (m, 2H), 4.56 (d, 1H), 4.45-4.30 (m, 1H), 3.30-3.17 (m, 1H), 2.19 (s, 3H), 2.05-1.65 (m, 5H), 1.58-1.38 (m, 1H), 1.37-1.15 (m, 10H), 1.07-0.90 (m, 1H).

LC/MS: m/z=475.97 (M+H⁺).

Compound 16: 3-[(4-Chloro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 7.29 (d, 2H), 7.20 (d, 2H), 6.99 (s, 1H), 4.60 (d, 1H), 4.39-4.28 (m, 1H), 4.28-4.19 (m, 1H), 3.29-3.17 (m, 1H), 3.15 (d, 2H), 1.97-1.82 (m, 3H), 1.82-1.71 (m, 1H), 1.29-1.20 (m, 9H), 1.01-0.86 (m, 2H).

LC/MS: m/z=460.01 (M+H⁺).

Compound 24: 5-(3,3-Dimethyl-but-1-ynyl)-3-[(3-fluoro-4-methyl-benzoyl)-(trans-4-hydroxy-cyclohexyl)-amino]-thiophene-2-carboxylic acid

LC/MS: m/z=458.11 (M+H⁺).

### Compound 52: 3-[(4-Chloro-3-fluoro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 13.59-13.40 (m, 1H), 7.48 (t, 1H), 7.38 (s, 1H), 7.20 (d, 1H), 7.03 (d, 1H), 4.55 (d, 1H), 4.43-4.32 (m, 1H), 3.31-3.21 (m, 1H), 2.02-1.71 (m, 5H), 1.51-1.39 (m, 2H), 1.29 (s, 9H), 1.27-1.17 (m, 1H), 1.08-0.94 (m, 1H).

LC/MS: m/z=478.06 (M+H⁺).

### Preparation of 3-[(2,4-dichloro-benzoyl)-(tetrahydro-pyran-4-yl)-amino]-5-(3,3-dimethyl-but-1-ynyl)-thiophene-2-carboxylic acid (26)

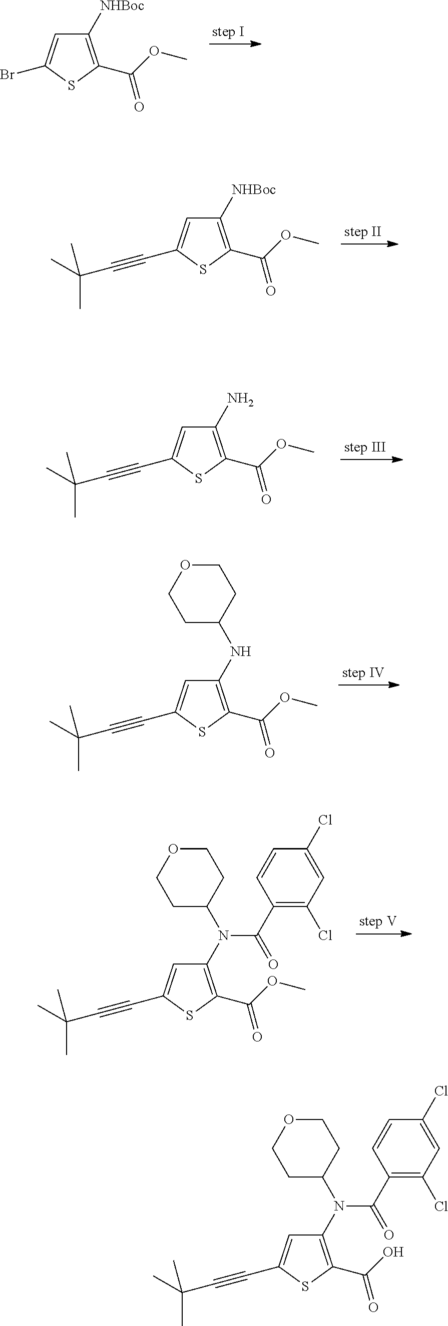

### Step I:

To a solution of 3-(tert-butoxycarbonyl)amino-5-bromo-thiophene-2-carboxylic acid methyl ester (4.566 g, 13.58 mmol) in dry DMF (40 mL) were added copper (I) iodide (52 mg, 0.27 mmol), Pd₂ dba₃ (622 mg, 0.68 mmol) and triethylamine (9.46 mL, 67.9 mmol). The mixture was deoxygenated by bubbling nitrogen through the solution for 10 minutes. tert-Butylacetylene (6.62 mL, 54.32 mmol) and BINAP (676 mg, 1.09 mmol) were then added to the mixture and heated at 60° C. overnight under nitrogen. The mixture was diluted with dichloromethane and filtered through celite washing with dichloromethane. The filtrate was washed with brine and the organic fraction was collected, dried over Na₂SO₄, concentrated and the residue was purified by silica gel chromatography eluting with a gradient of EtOAc in hexanes (0 to 50%) to give 5-(3,3-dimethyl-but-1-ynyl)-3-(tert-butoxycarbonyl)amino-thiophene-2-carboxylic acid methyl ester (4.364 g, 95%).

### Step II:

To a solution of 5-(3,3-dimethyl-but-1-ynyl)-3-(tert-butoxycarbonyl)amino-thiophene-2-carboxylic acid methyl ester (4.344 g, 9.58 mmol) in dichloromethane (30 mL) was added trifluoroacetic acid (30 mL), and the mixture was stirred at room temperature overnight. The mixture was evaporated to dryness and the residue was dissolved in dichloromethane and washed with aqueous NaHCO₃ and brine. The organic fraction was separated, dried over Na₂SO₄, and concentrated to give 3.135 g of crude 5-(3,3-dimethyl-but-1-ynyl)-3-amino-thiophene-2-carboxylic acid methyl ester.

### Step III:

To a solution of 5-(3,3-dimethyl-but-1-ynyl)-3-amino-thiophene-2-carboxylic acid methyl ester (0.500 g, 2.11 mmol) and tetrahydro-pyran-4-one (421 mg, 4.21 mmol) in toluene (3 mL) were added acetic acid (120 μL, 2.10 mmol) and sodium triacetoxyborohydride (886.0 mg, 4.18 mmol) under nitrogen atmosphere. The reaction mixture was stirred at RT overnight, filtered and washed with toluene (10 mL). The organic layer was washed with saturated sodium bicarbonate solution (1×10 mL), and EtOAc (1×20 mL) and concentrated to dryness. The residue was purified by silica gel chromatography (0-50% ethyl acetate with hexanes) to obtain 5-(3,3-dimethyl-but-1-ynyl)-3-(tetrahydro-pyran-4-ylamino)-thiophene-2-carboxylic acid methyl ester (388 mg, 57%).

### Step IV:

2,4-Dichloro-benzoyl chloride (48 mg, 0.23 mmol) was added to a solution of 5-(3,3-dimethyl-but-1-ynyl)-3-(tetrahydro-pyran-4-ylamino)-thiophene-2-carboxylic acid methyl ester (50 mg, 0.155) in dichloroethane (1 mL). The mixture was refluxed for 16 hr, and then it was brought to room temperature. Then the mixture was diluted with dichloromethane, washed with brine and the organic fraction was separated, dried over Na₂SO₄, and concentrated. The residue was purified by silica gel column chromatography using a gradient of ethyl acetate in hexanes (0% to 50%) to obtain 3-[(2,4-dichloro-benzoyl)-(tetrahydro-pyran-4-yl)-amino]-5-(3,3-dimethyl-but-1-ynyl)-thiophene-2-carboxylic acid methyl ester (70 mg, 91%).

### Step V:

To a solution of 3-[(2,4-dichloro-benzoyl)-(tetrahydro-pyran-4-yl)-amino]-5-(3,3-dimethyl-but-1-ynyl)-thiophene-2-carboxylic acid methyl ester from step 1V (70 mg, 0.14 mmol) in THF:H₂O (2 mL, 4:1) was added lithium hydroxide monohydrate (32 mg, 0.76 mmol) under nitrogen atmosphere. The reaction mixture was heated at 50° C. for 3 hr, cooled to RT, and concentrated to ⅓ of its volume. The reaction mixture was diluted with dichloromethane and acidified to pH 3 with HCl 1N. The reaction mixture was extracted with dichloromethane, and the organic layer was washed with brine, dried over Na₂SO₄, filtered and concentrated to dryness. The residue was purified by silica gel column chromatography (0 to 10% methanol in dichloromethane) to give 3-[(2,4-dichloro-benzoyl)-(tetrahydro-pyran-4-yl)amino]-5-(3,3-dimethyl-but-1-ynyl)-thiophene-2-carboxylic acid (32 mg, 48%).

LC/MS: m/z 482.00 (M+H⁺). (RT: 15.10, conditions: 5-85% CH₃CN in water with 0.01% TFA in 20 minutes run time. Column: Symmetry Shield RP18 2.1×50 mm and particle size 3.5 micro).

### 4-[[2-Carboxy-5-(3,3-dimethyl-but-1-ynyl)-thiophen-3-yl]-(2,4-dichloro-benzoyl)-amino]-1-methyl-piperidinium chloride (95)

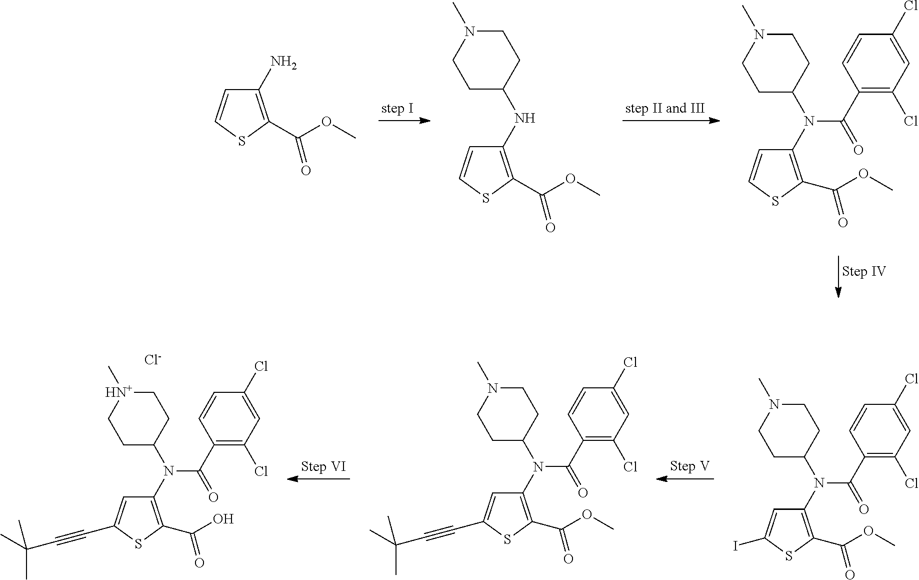

### Step I:

To a solution of 3-amino-thiophene-2-carboxylic acid methyl ester (10 g, 63.6 mmol) and 1-methyl-piperidin-4-one (12.9 g, 110 mmol) in dichloroethane (100 mL) was added sodium triacetoxyborohydride (23 g, 110 mmol) and acetic acid (6.6 g, 110 mmol). The resulting suspension was stirred at 20-25° C. under N₂ (g) for 24 hr. The reaction was quenched by water (25 mL) and the mixture is stirred for 1 h. The dichloroethane layer was separated and treated with water (75 mL) again. The mixture was stirred for another 1 hr and the organic layer was separated. To the dichloroethane layer was added water and NaHCO₃ solid was charged portion wise while stirring for 20 minutes. The final pH was between 8 and 9. The organic layer was separated, dried over Na₂SO₄ and evaporated to dryness. The residue was purified by silica gel chromatography to give 3-(1-methyl-piperidin-4-ylamino)-thiophene-2-carboxylic acid methyl ester (2.8 g, 18% yield).

### Step II

To a solution of 3-(1-methyl-piperidin-4-ylamino)-thiophene-2-carboxylic acid methyl ester (2.8 g, 11 mmol) in dichloroethane (10 mL) was added 2,4-dichloro-benzoyl chloride (2.69 g, 13.2 mmol) and the solution was heated at 80° C. overnight. The heating bath was removed and the reaction mixture was cooled to room temperature. Dichloromethane (75 mL) was added and stirred the suspension for 30 minutes in an ice bath. The suspension was filtered to obtain the hydrochloride salt of 3-[(2,4-dichloro-benzoyl)-(1-methyl-piperidin-4-yl)-amino]-thiophene-2-carboxylic acid methyl ester (4.0 g, 79% yield).

### Step III

To a stirring suspension of hydrochloride salt of 3-[(2,4-dichloro-benzoyl)-(1-methyl-piperidin-4-yl)-amino]-thiophene-2-carboxylic acid methyl ester (3.0 g, 6.49 mmol) in dichloromethane (30 mL) was added a saturated solution of sodium bicarbonate (10 mL). The mixture was stirred for 30 minutes, then the organic fraction was separated, dried over Na₂SO₄ and concentrated to dryness to afford 3-[(2,4-dichloro-benzoyl)-(1-methyl-piperidin-4-yl)-amino]-thiophene-2-carboxylic acid methyl ester (2.7 g, 98% yield).

### Step IV

To a solution of diisopropylamine (0.6 ml, 4.21 mmol) in anhydrous THF (3 mL) was added n-butyllithium (1.6M in Hexanes, 2.34 mL, 3.74 mmol) at −40° C. The reaction mixture was stirred at −40° C. for 15 minutes and then cooled to −78° C. 3-[(2,4-dichloro-benzoyl)-(1-methyl-piperidin-4-yl)-amino]-thiophene-2-carboxylic acid methyl ester (1 g, 2.34 mmol) was dissolved in anhydrous THF (13 mL) and then this solution was slowly added to the LDA solution over 30 minutes while keeping the temperature about −78° C. After complete addition, the solution was stirred for 30 minutes.

A solution of iodine (944 mg, 3.74 mmol) in anhydrous THF (2 mL) was added to the anion and stirred at −78° C. for 1 hr. The reaction mixture was allowed to rise to room temperature and then quenched with a saturated solution of ammonium chloride. The phases were separated and the aqueous phase was extracted with ethyl acetate. The organic layer was washed with a 5% solution of sodium thiosulfate and then with brine. The organic layer was dried over sodium sulfate, filtered and evaporated to dryness. The product was purified by silica gel chromatography to yield 3-[(2,4-dichloro-benzoyl)-(1-methyl-piperidin-4-yl)-amino]-5-iodo-thiophene-2-carboxylic acid methyl ester (200 mg, 15% yield).

### Step V

To a solution 3-[(2,4-dichloro-benzoyl)-(1-methyl-piperidin-4-yl)-amino]-5-iodo-thiophene-2-carboxylic acid methyl ester (200 mg, 0.36 mmol) in dry DMF (2 mL) were added copper (I) iodide (2 mg, 0.009 mmol), Pd₂ dba₃ (3 mg, 0.004 mmol) and triethylamine (125 uL, 0.9 mmol). The mixture was deoxygenated by bubbling nitrogen through solution for 10 minutes. tert-Butylacetylene (89 uL, 0.72 mmol) was added to the mixture and the tube was sealed. The mixture was stirred overnight at room temperature. The mixture was diluted with ethyl acetate and water, stirred for 5 minutes. The organic fraction was separated, dried over Na₂SO₄, concentrated, and the residue was purified by silica gel chromatography to give 140 mg (77%) of 3-[(2,4-dichloro-benzoyl)-(1-methyl-piperidin-4-yl)-amino]-5-(3,3-dimethyl-but-1-ynyl)-thiophene-2-carboxylic acid methyl ester.

### Step VI

The product from Step V (140 mg, 0.276 mmol) was hydrolysed with lithium hydroxide monohydrate (63 mg, 1.5 mmol) as previously described to give after HPLC purification 86 mg (63%) of 4-[[2-carboxy-5-(3,3-dimethyl-but-1-ynyl)-thiophen-3-yl]-(2,4-dichloro-benzoyl)-amino]-1-methyl-piperidinium chloride.

¹H NMR (400 MHz, DMSO-d6): δ [ppm] 7.55 (s, 1H), 7.39-7.22 (m, 2H), 7.15 (s, 1H), 4.75-4.61 (m, 1H), 3.50-2.90 (m, 4H), 2.69 (s, 3H), 2.30-1.50 (m, 4H), 1.29 (s, 9H).

LC/MS: m/z 494.73 (M+H⁺).

### Preparation 3-[(2,4-Dichloro-benzoyl)-(2-methoxy-ethyl)-amino]-5-(3,3-dimethyl-but-1-ynyl-1)-thiophene-2-carboxylic acid (44)

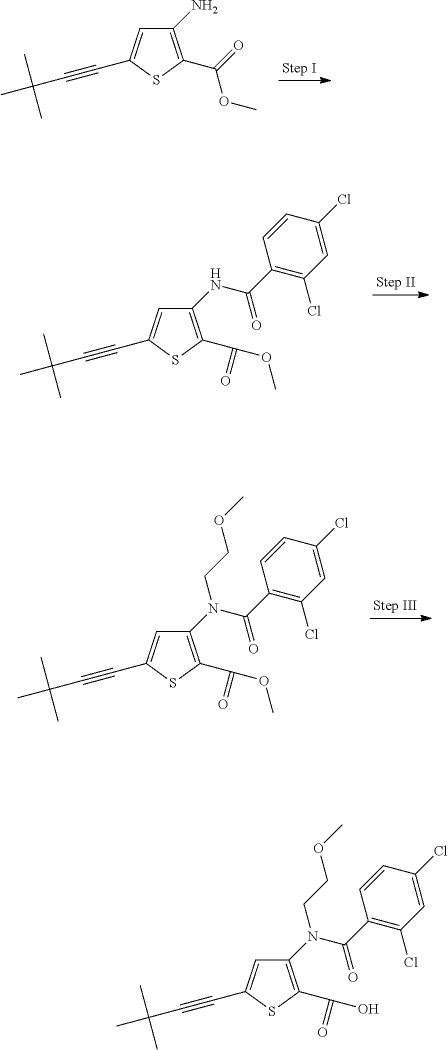 [SMILES: COC(=O)c1sc(C#CC(C)(C)C)cc1N ; COC(=O)c1sc(C#CC(C)(C)C)cc1NC(=O)c1ccc(Cl)cc1Cl ; COCCN(C(=O)c1ccc(Cl)cc1Cl)c1cc(C#CC(C)(C)C)sc1C(=O)OC ; COCCN(C(=O)c1ccc(Cl)cc1Cl)c1cc(C#CC(C)(C)C)sc1C(=O)O]

### Step I

To a solution of 3-amino-5-(3,3-dimethyl-but-1-ynyl)-thiophene-2-carboxylic acid methyl ester (250 mg, 1.05 mmol) in toluene (2 mL) was added pyridine (100 mg, 1.23 mmol) and 2,4-dichloro-benzoyl chloride (329 mg, 1.58 mmol) under nitrogen atmosphere. The reaction mixture was heated at 100° C. overnight and then cooled to room temperature. A saturated solution of sodium bicarbonate was added to the reaction mixture. The organic fraction was separated, dried over Na₂SO₄ and concentrated to dryness. The residue was purified by silica gel chromatography using 0-30% ethyl acetate/hexanes as eluant to give 3-(2,4-dichloro-benzoylamino)-5-(3,3-dimethyl-but-1-ynyl)-thiophene-2-carboxylic acid methyl ester (300 mg, 70%).

### Step II

To a solution of 3-(2,4-dichloro-benzoylamino)-5-(3,3-dimethyl-but-1-ynyl)-thiophene-2-carboxylic acid methyl ester (150 mg, 0.36 mmol) in DMF (2 mL) at 0° C. was added sodium hydride 60% in mineral oil (29 mg, 0.72 mmol) under nitrogen atmosphere. The mixture was stirred in an ice bath for 10 minutes then 1-bromo-2-methoxy-ethane (75 mg, 0.54 mmol) was added. The reaction mixture was stirred at room temperature overnight and quenched with H₂O (2 mL). The reaction mixture was extracted by EtOAc (2×5 mL), and the organic phase was dried over Na₂SO₄, filtered, and concentrated to dryness. The residue was purified by silica gel chromatography using 0-50% ethyl acetate/hexanes as eluant to give 3-[(2,4-dichloro-benzoyl)-(2-methoxy-ethyl)-amino]-5-(3,3-dimethyl-but-1-ynyl)-thiophene-2-carboxylic acid methyl ester (60 mg, 35% yield).

### Step III

To a solution of 3-[(2,4-dichloro-benzoyl)-(2-methoxy-ethyl)-amino]-5-(3,3-dimethyl-but-1-ynyl)-thiophene-2-carboxylic acid methyl ester (60 mg, 0.128 mmol) in a 3:2:1 mixture of THF:methanol:H₂O (2 mL) was added lithium hydroxide monohydrate (9 mg, 0.214 mmol) under nitrogen atmosphere. The reaction mixture was stirred at RT overnight, acidified to pH 3-4 with 1N aqueous HCl. The reaction mixture was extracted by EtOAc (2×5 mL), and the organic phase was dried over Na₂SO₄, filtered, and concentrated to dryness. The residue was purified by silica gel chromatography using 0-10% dichloromethane/methanol as eluant to give 3-[(2,4-dichloro-benzoyl)-(2-methoxy-ethyl)-amino]-5-(3,3-dimethyl-but-1-ynyl)-thiophene-2-carboxylic acid (44 mg, 76% yield) as a mixture of two rotamers (9:1).

¹H NMR for major rotamer: (400 MHz, DMSO-d6): 6 [ppm] 7.50 (d, 1H), 7.42 (d, 1H), 7.28 (dd, 1H), 6.92 (s, 1H), 4.23-4.10 (m, 1H), 3.52-3.15 (m, 3H), 3.18 (s, 3H), 1.18 (s, 9H).

LC/MS: m/z 455.98 (M+H⁺). (RT: 15.17, conditions: 5-85% CH₃CN in water with 0.01% TFA in 20 minutes run time. Column: Symmetry Shield RP18 2.1×50 mm and particle size 3.5 micro).

### Preparation of 3-[(2,4-Dichloro-benzoyl)-(2-hydroxy-ethyl)-amino]-5-(3,3-dimethyl-but-1-ynyl)-thiophene-2-carboxylic acid (97)

The compound is synthesized by following the similar procedures reported for compound 44.

LC/MS: m/z 441.82 (M+H⁺). (RT: 13.65, conditions: 5-85% CH₃CN in water with 0.01% TFA in 20 minutes run time. Column: Symmetry Shield RP18 2.1×50 mm and particle size 3.5 micro).

### 3-[(2,4-Dichloro-benzoyl)-isopropyl-amino]-5-(3,3-dimethyl-but-1-ynyl)-thiophene-2-carboxylic acid (1)

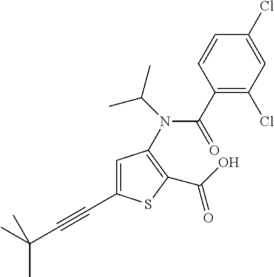 [SMILES: CC(C)N(C(=O)c1ccc(Cl)cc1Cl)c1cc(C#CC(C)(C)C)sc1C(=O)O]

¹H NMR (400 MHz, DMSO-d6): δ [ppm] 7.54 (d, 1H), 7.35 (dd, 1H), 7.27 (d, 1H), 7.24 (s, 1H), 4.84-4.68 (m, 1H), 1.28 (d, 3H), 1.27 (s, 9H), 0.96 (d, 3H).

### Sodium 3-[(2,4-dichloro-benzoyl)-isopropyl-amino]-5-(3,3-dimethyl-but-1-ynyl)-thiophene-2-carboxylate (96)

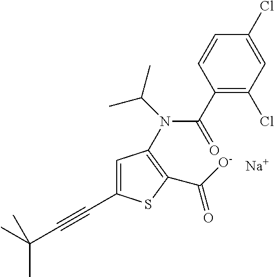 [SMILES: CC(C)N(C(=O)c1ccc(Cl)cc1Cl)c1cc(C#CC(C)(C)C)sc1C(=O)[O-] ; [Na+]]

3-[(2,4-Dichloro-benzoyl)-isopropyl-amino]-5-(3,3-dimethyl-but-1-ynyl)-thiophene-2-carboxylic acid (11.82 mg, 0.027 mmol) was taken in a mixture of water (3 mL) and CH₃CN (0.5 mL) and a solution of sodium hydroxide (53.3 uL, 0.5065 N, 0.027 mmol) was added to it. The solution was stirred for 30 minutes and then lyophilized to obtain sodium 3-[(2,4-dichloro-benzoyl)-isopropyl-amino]-5-(3,3-dimethyl-but-1-ynyl)-thiophene-2-carboxylate (12.4 mg, 100%).

### Example 3

### Evaluation of Compounds in the HCV RNA-Dependent RNA Polymerase Assay

The following references are all incorporated by reference:
1. Behrens, S., Tomei, L., De Francesco, R. (1996) EMBO 15, 12-22
2. Harlow, E, and Lane, D. (1988) Antibodies: A Laboratory Manual. Cold Spring Harbord Laboratory. Cold Spring Harbord. NY.
3. Lohmann, V., Körner, F., Herian, U., and Bartenschlager, R. (1997) J. Virol. 71, 8416-8428
4. Tomei, L., Failla, C., Santolini, E., De Francesco, R., and La Monica, N. (1993) J Virol 67, 4017-4026
5. Ferrari, E., Wright-Minogue, J., Fang, J. W., Baroudy, B. M., Lau, J. Y., and Hong, Z. Characterization of soluble hepatitis C virus RNA-dependent RNA polymerase expressed in Escherichia coli. J. Virol. 1999, 73, 1649-1654.
6. Lesburg, C. A; Cable, M. B; Ferrari, E; Hong, Z; Mannarino, A. F; Weber, P.C. Crystal structure of the RNA-dependent RNA polymerase from hepatitis C virus reveals a fully encircled active site. Nature Struct. Biol. 1999, 6, 937-943

Compounds can be evaluated using an in vitro polymerase assay containing purified recombinant HCV RNA-dependent RNA polymerase (NS5B protein) expressed in E. coli cells. The experimental procedures used for the cloning, expression and purification of the HCV NS5B protein are described below. Follows, are details of the RNA-dependent RNA polymerase assays for testing the compounds.

### Example 4

### Cloning and Expression of the HCV NS5B Protein in E. Coli Cells

The cDNA encoding the entire NS5B protein of HCV-Bk strain, genotype 1b, was amplified by PCR using the primers NS5Nhe5′ (5′-GCTAGCGCTAGCTCAATGTCCTACACATGG-3′ (SEQ ID NO: 1)) and XhoNS53′ (5′-CTCGAGCTCGAGCGTCCATCGGTTGGGGAG-3′ (SEQ ID NO: 2)) and the plasmid pCD 3.8-9.4 as template (Tomei et al, 1993). NS5Nhe5′ and XhoNS53′ contained two Nhe I and Xho I sites (underlined sequences), respectively, at their 5′ end. The amplified DNA fragment was cloned in the bacterial expression plasmid pET-21b (Novagen) between the restriction sites Nhe I and Xho I, to generate the plasmid pET/NS5B. This plasmid was later used as template to PCR-amplify the NS5B coding region minus the 21 amino acid carboxy terminal region and with the addition of a N-terminal histidine tag using the primers NHISNSSB (5′-GCT AGG GCT AGC CAC CAC CAC CAC CAC CAC TCA ATG TCC TAC ACA TGG ACA-3′ (SEQ ID NO: 3)) and. HCV NS5BTR1 (5′-CTC GAG CTC GAG TCA ACG GGG TCG GGC ACG AGA CAG-3′ (SEQ ID NO: 4)). The PCR fragment was cloned once again into the pET-21b expression plasmid which was used express the truncated form of the histidine-tagged hCV polymerase in Escherichia coli BL21 (DE3) (Invitrogen Life Technologies, Carlsbad, USA) according to the manufacturers' protocol.

### Example 5

### Purification of Recombinant NS5B Using Fast Protein Liquid Chromatography (FPLC)

The truncated enzyme was purified as described in Lesburg et al. and Ferrari et al. with minor modifications. Briefly, soluble bacterial lysates were loaded onto a HiTrap nickel chelating affinity column (GE Healthcare, Baie d'Urfe, Canada). The bound enzyme was eluted using an imidazole gradient from 10 to 500 mM. Imidazole was then removed from the buffer of the pooled active fractions using PD-10 desalting columns (GE Healthcare). Further purification was achieved by running the protein preparation through a cation exchange HiTrap SP sepharose column (GE Healthcare) using a 300 to 1000 mM NaCl gradient to elute the protein. Thereafter, buffer was changed to 10 mM Tris pH 7.5, 10% glycerol, 5 mM DTT, 600 mM NaCl using a PD-10 column. Positive fractions were tested for RNA-dependent polymerase activity and the most active fractions are pooled and stored at −80° C.

### Example 6

### In Vitro HCV RdRp Scintillation Proximity Assay (SPA) Used to Evaluate Analogues

A 15-mer 5′ biotinylated DNA oligonucleotide (oligo dT) primer (dT15″ disclosed as SEQ ID NO: 5) annealed to a homopolymeric poly rA RNA template was captured on the surface of streptavidin-coated bead (GE Healthcare) and the polymerization activity of the HCV NS5B enzyme was quantified by measuring the incorporation of radiolabeled [3H] UTP substrate onto the growing primer 3′ end. In brief, a 400 ng/μl poly rA solution (Amersham Pharmacia Biotech) was mixed volume-to-volume with 5′ biotin-oligo dT15 at 20 pmol/μl (dT15″ disclosed as SEQ ID NO:5). The template and primers were denatured at 95° C. for 5 minutes then incubated at 37° C. for 10 minutes. Compounds were added in a 10 μl solution followed by a mix containing the following for a final concentration of: 10 mM MgCl₂, 20 mM Tris-HCl pH 7.5, 50 mM NaCl, 1 mM DTT and 250 ng of the poly rA/oligo dT template. The enzymatic reaction was initiated upon addition of enzyme and substrate to obtain the following concentrations: 1 μM UTP, 1 μCi [3H] UTP and 50 nM recombinant HCV NS5B. Detection of incorporated radioactivity was achieved by counting the signal using a liquid scintillation counter (Wallac Microbeta Trilux, Perkin Elmer, Mass., USA).

IC₅₀ values of certain tested compounds of the invention obtained by this assay are summarized in Table 2 (see column entitled “HCV_Polymerase_{—}1b”):
A: IC₅₀ value (mean)≦0.1 μM;
B: 0.1 μM<IC₅₀ value (mean)≦1 μM;
C: 1 μM<IC₅₀ value (mean)≦10 μM;
D: IC₅₀ value (mean)>10 μM.

### Example 7

### HCV Replicon Assay

### A. Principle

This procedure below describes the HCV replicon assay using a Huh7 hepatoma cell line harboring a highly cell culture-adapted replicon (genotype 1b) (hereafter named cell line ET). The ET cells contained the highly cell culture-adapted replicon I₃₈₉luc-ubi-neo/NS3′/5.1 construct that carried, in addition to the neomycin gene, an integrated copy to the firefly luciferase gene (Krieger, N; Lohmann, V; Bartenschlager, R. Enhancement of hepatitis C virus RNA replication by cell culture-adaptive mutations. J. Virol. 2001, 75, 4614-4624). A replicon cell line W11.8, containing the 1a genotype of HCV was also used. These two cell lines (genotype 1b and 1a) allowed measurement of RNA replication and translation by measuring luciferase activity (against genotype 1b) or by measuring the NS5A level using the ELISA assay (against genotype 1a). It was shown that the luciferase activity tightly followed the replicon RNA level in the ET cells. ET cell lines were maintained in cultures at a sub-confluent level (<85%). The culture media used for cell passaging consisted of DMEM (Gibco BRL Laboratories, Mississauga, ON, Canada) supplemented with 10% fetal bovine serum with 1% penicillin/streptomycin, 1% glutamine, 1% sodium pyruvate, 1% non-essential amino acids, and 180 μg/ml of G418 final concentration.

### B. Measurement of Luciferase Activity (Luci-ET-1b)

For the treatment of the cells with the testing drug, the culture medium was removed from the 175 cm² T-flask by aspiration. Cell monolayer was rinsed with 10 mL of PBS 1× at room temperature. PBS was removed by aspiration. Cells were trypsinized using 1 mL of Trypsin/EDTA. Flask were incubated at 37° C. (incubator) for 7 minutes. Complete medium (9 mL) with no G418 and no phenol red was then added. Cell clumps were disrupted by pipetting up and down several times. The cell suspension was then transferred to a 50 mL Falcon polypropylene tube. Cells were then counted several times using the hemacytometer. Cells were diluted at 30 000 cells/mL with complete DMEM with no G418 and no phenol red, then transferred into a sterile reservoir. Using a multichannel pipet, approximately 3000 viable cells (100 μL) were plated per well in a white opaque 96-well microtiter plate. After an incubation period of 2-4 hours at 37° C. in a 5% CO₂ incubator, compounds were added at various concentrations.

Compounds under testing were resuspended in DMSO at a stock concentration of 100 mM. Then, they were diluted at twice the final concentration in the same medium (without G418) described earlier, in sterile 96-deep well plate and according to a particular template. One volume (100 μL) of each compound dilution was then added to each well that contains cells or in control wells with no cells. Final drug concentrations were usually between 200 μM and 0.0001 μM. Ten wells were used as positive control without drug. Cells were further incubated for 4 days at 37° C. in a 5% CO₂ incubator. A control compound was used as an internal standard at the same concentrations described above.

Following the incubation time of four days, the culture media was removed and quickly dried upside down on a stack of sterile absorbing papers. Cells were then lysed by the addition of 95 μL of the luciferase buffer A using a mutichannel pipet, sealed using TopSeal™ adhesive sealing film and the reaction mixture was incubated at room temperature and protected from direct light for at least 10 minutes. Plates were read for luciferase counts using a luminometer (Wallac MicroBeta Trilux, Perkin Elmer™, MA, USA).

The percentage of inhibition at each drug concentration tested (in duplicate) was calculated. The concentration required to reduce viral replication by 50% (IC₅₀) was then determined from dose response curves using nonlinear regression analysis (e.g., GraphPad Prism software, version 2.0 (GraphPad Software Inc., San Diego, Calif., USA)). The IC50 values are summarized in Table 2:
A: IC₅₀ value (mean)≦0.1 μM;
B: 0.1 μM<IC₅₀ value (mean)≦1 μM;
C: 1 μM<IC₅₀ value (mean)≦10 μM;
D: IC₅₀ value (mean)>10 μM.

### C. Elisa Assay (ELISA W 11.8-1a)

Replicon cell lines W11.8 containing a sub-genomic replicon of genotype 1a was used for the HCV Replicon Cell-Based detection using the ELISA. The RNA replication in presence of different drug concentrations was indirectly measured in these cell lines by the level of NS5A protein content upon drug treatment for four days. The NS5A is a non-structural protein of HCV and is used as marker of HCV replication in this assay.

For the treatment of the cells with the testing drug, Culture medium was removed from the 175 cm² T-flask by aspiration. Cell monolayer was rinsed with 10-20 mL of PBS 1× at room temperature. PBS was removed by aspiration. Cells were trypsinized using 3 mL of Trypsin (0.25%)/EDTA (0.1%) solution. Flasks were incubated at 37° C. (incubator) for 7 minutes. Complete medium (9 mL) without G418 is then added. Cell clumps were disrupted by pipetting up and down several times.

The cell suspension was then transferred to a 50 mL Falcon polypropylene tube. Cells were then counted several times using the haemocytometer. Cells were diluted at 50,000 cells/mL with complete DMEM without G418, then transferred into a sterile reservoir. Using a multichannel pipet, approximately 5,000 viable cells (100 μL) were plated per well in a white opaque 96-well microtiter plate. After an incubation period of 2-4 hours at 37° C. in a 5% CO₂ incubator, compounds were added at various concentrations.

Drugs were resuspended in DMSO at a stock concentration of 100 mM or 10 mM. In some cases (drugs with a potency below nmolar values), it was necessary to dilute compounds in DMSO at 1 mM or 100 μM as a starting solution. Then, drugs were diluted at twice the final concentration in the same medium (without G418) described earlier, in sterile 96-deep well plate and according to a particular template (see Appendix). One volume (100 μL) of each drug dilution was then added to each well that contains cells.

Sixteen wells were used as control (0% inhibition) without drug. Eight wells were used as background control (100% inhibition) containing 2 μM (final concentration) of the reference compound. The reference compound at 2 μM was shown to inhibit the NS5A expression at ≈100% and is nontoxic to the cells. Values from 100% inhibited wells were averaged and used as the background value. Cells are further incubated for 4 days at 37° C. in a 5% CO₂ incubator.

For the measurement of NS5A protein content, following the incubation time of four days, the media was throwed into an appropriate waste container by inverting the plate. Any residual liquid was removed by tapping gently on absorbent paper several times. The plates were then washed once with 150 μL of PBS per well, and then incubated for 5 minutes at room temperature on a shaker (500 rpm). 150 μL per well of cold (−20° C.) fixative solution (50% methanol/50% acetone mix) was added into the plates, and the plates was incubated for 5 minutes at room temperature. The pleates were then inverted, and any residual liquid was removed by tapping gently on absorbent paper several times. The plates were then washed twice with 150 μL of PBS per well, and incubated for 5 minutes at room temperature on a shaker (500 rpm) for each wash. 150 μL of blocking solution per well was added into the plates. The plates were then sealed is using TopSeal™ adhesive sealing films and incubated for one hour at 37° C. or at 4° C. overnight to block non-specific sites.

The plates were invered and the blocking solution was dumped into an appropriate waste container. Any residual liquid was removed by tapping gently on absorbent paper several times. The plates were then washed twice with 150 μL of PBS per well and once with 150 μL of PBSTS solution per well, and then incubated for 5 minutes at room temperature on a shaker (500 rpm) for each wash. Then, was add into the plates 50 μL per well of anti-human NS5A antibody (Ab1) diluted 1/1,000 in the blocking solution. The plates were then sealed using TopSeal™ adhesive sealing films and incubate at 4° C. overnight.

Next day, the plates were invered to dump solution into an appropriate waste container. The plates then were gently tapped on absorbent paper several times to remove residual liquid. The plates were washed five times with 150 μL of PBS per well, and incubated for 5 minutes at room temperature on a shaker (500 rpm) for each wash. Then was add into the plates 50 μL per well of peroxidase-conjugated donkey anti-mouse antibody (Ab2) diluted 1/10,000 in the blocking solution. The plates were then sealed using TopSeal™ adhesive sealing films and incubate at room temperature for 3 hours on a shaker (500 rpm). Towards the end of the 3 hours incubation, the commercially available chemiluminescent substrate solution was prepared. A mixture of equal volumes of the luminol/enhancer and stable peroxide reagents was prepared and protected from light. The plates were then inverted to dump solution into an appropriate waste container. Any residual liquid was removed by tapping gently on absorbent paper several times. The plates were washed four times with 150 μL of PBSTS solution per well and once with 150 μL of PBS, and then incubated for 5 minutes at room temperature on a shaker (500 rpm) for each wash. 100 μL of substrate solution per well was then added into the plates. The plates were then sealed using TopSeal™ adhesive sealing films and incubate for 1 minute at room temperature on a shaker (500 rpm), and then incubated between 30 and 60 minutes at room temperature (protect from light) prior to reading the luminescence (relative light units) on the Analyst HT plate reader (LJL Default Luminescence Method).

The percentage of inhibition at each drug concentration tested (in duplicate) was calculated. The concentration required to reduce viral replication by 50% (IC₅₀) was then determined from dose response curves using nonlinear regression analysis (e.g., GraphPad Prism software, version 2.0 (GraphPad Software Inc., San Diego, Calif., USA)). The IC50 values are summarized in Table 2:
A: IC₅₀ value (mean)≦0.1 μM;
B: 0.1 μM<IC₅₀ value (mean)≦1 μM;
C: 1 μM<IC₅₀ value (mean)≦10 μM;
D: IC₅₀ value (mean)>10 μM.

### Example 8

### [³H]Thymidine Incorporation Assay (CC50)

A total of 2,000 cells/well were seeded in 96-well cluster dishes in a volume of 100 μL of DMEM (Wisent., St Bruno, QC) supplemented with 10% FBS (Wisent., St Bruno, QC) and 2 mM glutamine (Life Technologies, Inc.). Penicillin and streptomycin (Life Technologies, Inc.) were added to 500 U/mL and 50 μg/mL final concentrations, respectively. After an incubation of at least 3 h at 37° C. in an atmosphere of 5% CO₂, compounds, prepared at twice the final concentration, were added to the cells. Eleven serial two to four-fold dilutions of drugs were tested in duplicate plates. After 72-h incubation, a volume of 20 μL of a 10 μCi/mL solution of [3H] methyl thymidine (Amersham Life Science, Inc., Arlington Heights, III.; 2 Ci/mmol) in culture medium was added and the plates were incubated for a further a 24 h at 37° C. Cells were then washed with phosphate-buffered saline (PBS), trypsinized for 2 min, and collected onto a fiberglass filter using a Tomtec cell harvester (Tomtec, Orange, Conn.). Filters were dried at 37° C. for 1 h and placed into a bag with 4.5 mL of liquid scintillation cocktail (Wallac Oy, Turku, Finland). The accumulation of [3H] methyl thymidine, representing viable replicating cells, was measured using a liquid scintillation counter (1450-Microbeta; Wallac Oy). Ref. SOP: 265-162-03. For this experiment, the cell lines used were; Huh-7 ET (cells derived from the Huh-7 cell line (hepatocellular carcinoma, human) and containing an HCV sub-genomic replicon), Molt-4 (peripheral blood, acute lymphoblastic leukemia, human), DU-145 (prostate carcinoma, metastasis to brain, human), Hep-G2 (hepatocellular carcinoma, human), and SH-SYSY (neuroblastoma, human) cells.

The cytotoxic potential of the tested compounds was determined using [3H]-thymidine incorporation assay described above. The 50% cytotoxic concentrations (CC₅₀) for cell toxicity were determined from dose response curves using six to eight concentrations per compound in triplicate. Curves were fitted to data points using non-linear regression analysis. CC₅₀ values can be interpolated from the resulting curve (e.g., GraphPad Prism software, version 2.0 (GraphPad Software Inc., San Diego, Calif., USA)). CC₅₀ values of compounds of the invention are summaries in Table 2:
A: CC₅₀ value (mean)≧100 μM;
B: 10 μM≦CC₅₀ value (mean)<100 μM;
C: CC₅₀ value (mean)≦10 μM.

TABLE 2
IC₅₀, CC₅₀, LCMS and NMR Data of Certain Compounds of the Invention

| | | HCV- | HCV- | | | | |
|---|---|---|---|---|---|---|---|
| Com- | | Replicon- | Replicon- | HCV_{—} | | | |
| pound | | Luci-Et- | ELISA-1a | Polymerase_1b | | LCMS | |
| Nos. | Structure | 1b_IC50 | IC50 | IC50 | CC50 | [M + H]⁺ | NMR |
| 1 | 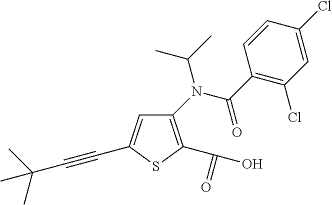 [SMILES: CC(C)N(C(=O)c1ccc(Cl)cc1Cl)c1cc(C#CC(C)(C)C)sc1C(=O)O] | B | | B | B | 440.0 | ¹H NMR (400 MHz, DMSO-d6): δ [ppm] 7.54 (d, 1H), 7.35 (dd, 1H), 7.27 (d, 1H), 7.24 (s, 1H), 4.84-4.68 (m, 1H), 1.28 (d, 3H), 1.27 (s, 9H), 0.96 (d, 3H) |
| 2 | 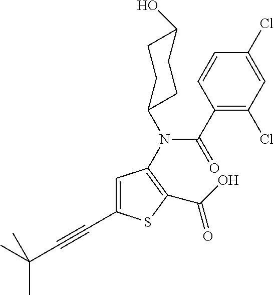 [SMILES: CC(C)(C)C#Cc1cc(N(C(=O)c2ccc(Cl)cc2Cl)C2CCC(O)CC2)c(C(=O)O)s1] | A | | B | B | 494.03 | ¹H NMR (400 MHz, DMSO-d₆): δ 13.71 (s, 1H), 7.54 (d, 1H), 7.35 (dd, 1H), 7.25 (d, 1H), 7.21 (s, 1H), 4.58 (s, 1H), 4.44-4.34 (m, 1H), 3.30-3.23 (m, 1H), 2.05-1.99 (m, 1H), 1.93-1.69 (m, 4H), 1.53-1.41 (m, 2H), 1.27 (s, 9H), 1.02-0.90 (m, 2H). |
| 16 | 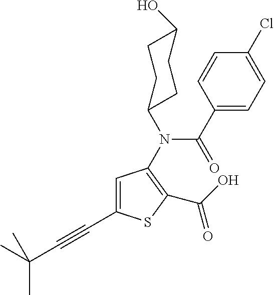 [SMILES: CC(C)(C)C#Cc1cc(N(C(=O)c2ccc(Cl)cc2)C2CCC(O)CC2)c(C(=O)O)s1] | B | | B | B | 460.01 | ¹H NMR (400 MHz, DMSO-d₆): δ 7.29 (d, 2H), 7.20 (d, 2H), 6.99 (s, 1H), 4.60 (d, 1H), 4.39-4.28 (m, 1H), 4.28-4.19 (m, 1H), 3.29-3.17 (m, 1H), 3.15 (d, 2H), 1.97-1.82 (m, 3H), 1.82-1.71 (m, 1H), 1.29-1.20 (m, 9H), 1.01-0.86 (m, 2H) |
| 17 | 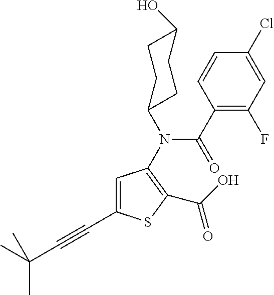 [SMILES: CC(C)(C)C#Cc1cc(N(C(=O)c2ccc(Cl)cc2F)C2CCC(O)CC2)c(C(=O)O)s1] | A | B | C | B | 478.04 | ¹H NMR (400 MHz, DMSO-d₆): δ 7.51 (m, 1H), 7.26 (m, 1H), 7.08 (m, 1H), 6.84 (s, 1H), 4.53 (bs, 1H), 4.34 (m, 1H), 3.25 (m, 1H), 1.95-1.84 (m, 3H), 1.82-1.73 (m, 1H), 1.44-1.12 (m, 12H), 0.99-0.84 (m, 1H) |
| 18 | 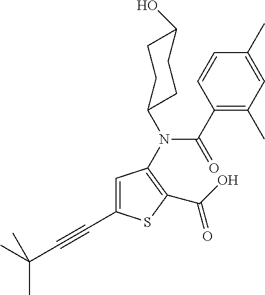 [SMILES: Cc1ccc(C(=O)N(c2cc(C#CC(C)(C)C)sc2C(=O)O)C2CCC(O)CC2)c(C)c1] | A | B | B | B | 454.13 | 1H NMR (400 MHz, DMSO-d6): δ 7.15- 7.01 (m, 2H), 6.86 (s, 1H), 6.72 (d, J = 7.7 Hz, 1H), 4.61-4.33 (m, 2H), 3.44-3.21 (m, 2H), 2.18 (d, J = 17.2 Hz, 6H), 2.03- 1.96 (d, J = 11.9 Hz, 1H), 1.90-1.75 (m, 3H), 1.45-1.28 (m, 3H), 1.25 (s, 9H), 0.99-0.85 (m, 1H) |
| 19 | 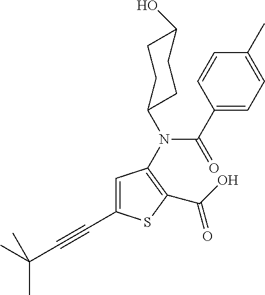 [SMILES: Cc1ccc(C(=O)N(c2cc(C#CC(C)(C)C)sc2C(=O)O)C2CCC(O)CC2)cc1] | A | | C | B | 440.13 | |
| 22 | 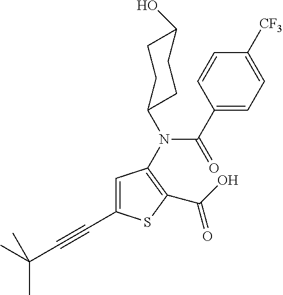 [SMILES: CC(C)(C)C#Cc1cc(N(C(=O)c2ccc(C(F)(F)F)cc2)C2CCC(O)CC2)c(C(=O)O)s1] | B | | C | B | 494.08 | |
| 24 | 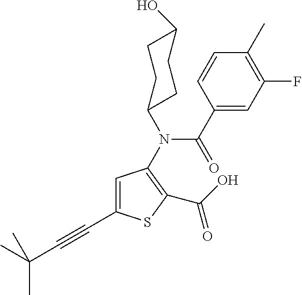 [SMILES: Cc1ccc(C(=O)N(c2cc(C#CC(C)(C)C)sc2C(=O)O)C2CCC(O)CC2)cc1F] | A | B | C | B | 458.11 | |
| 26 | 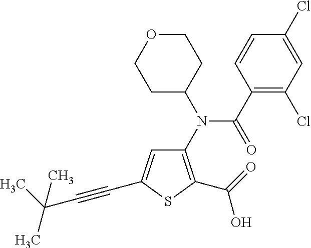 [SMILES: CC(C)(C)C#Cc1cc(N(C(=O)c2ccc(Cl)cc2Cl)C2CCOCC2)c(C(=O)O)s1] | A | | C | B | 482.00 | |
| 44 | 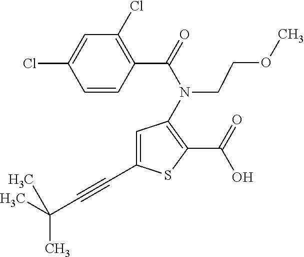 [SMILES: COCCN(C(=O)c1ccc(Cl)cc1Cl)c1cc(C#CC(C)(C)C)sc1C(=O)O] | A | B | B | B | 455.98 | ¹H NMR for major rotamer: (400 MHz, DMSO-d6): δ [ppm] 7.50 (d, 1H), 7.42 (d, 1H), 7.28 (dd, 1H), 6.92 (s, 1H), 4.23- 4.10 (m, 1H), 3.52- 3.15 (m, 3H), 3.18 (s, 3H), 1.18 (s, 9H) |
| 51 | 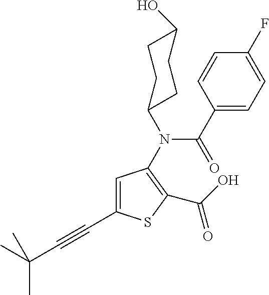 [SMILES: CC(C)(C)C#Cc1cc(N(C(=O)c2ccc(F)cc2)C2CCC(O)CC2)c(C(=O)O)s1] | C | | C | A | 444.09 | |
| 52 | 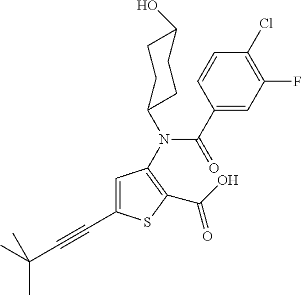 [SMILES: CC(C)(C)C#Cc1cc(N(C(=O)c2ccc(Cl)c(F)c2)C2CCC(O)CC2)c(C(=O)O)s1] | B | | C | B | 478.06 | ¹H NMR (400 MHz, DMSO-d₆): δ 13.59- 13.40 (m, 1H), 7.48 (t, 1H), 7.38 (s, 1H), 7.20 (d, 1H), 7.03 (d, 1H), 4.55 (d, 1H), 4.43-4.32 (m, 1H), 3.31-3.21 (m, 1H), 2.02-1.71 (m, 5H), 1.51-1.39 (m, 2H), 1.29 (s, 9H), 1.27- 1.17 (m, 1H), 1.08- 0.94 (m, 1H) |
| 53 | 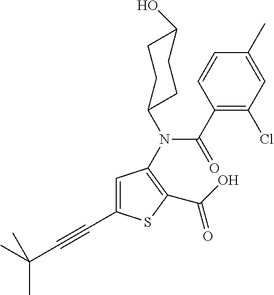 [SMILES: Cc1ccc(C(=O)N(c2cc(C#CC(C)(C)C)sc2C(=O)O)C2CCC(O)CC2)c(Cl)c1] | A | | C | B | 474.07 | |
| 54 | 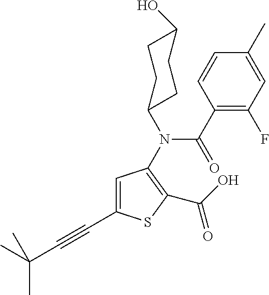 [SMILES: Cc1ccc(C(=O)N(c2cc(C#CC(C)(C)C)sc2C(=O)O)C2CCC(O)CC2)c(F)c1] | A | | C | B | 458.11 | ¹H NMR (400 MHz, DMSO-d₆): δ 13.48 (s, 1H), 7.17 (s, 1H), 7.10 (t, 1H), 6.86 (d, 2H), 4.55 (d, 1H), 4.45-4.32 (m, 1H), 3.31-3.20 (m, 1H), 2.22 (s, 3H), 2.00- 1.69 (m, 5H), 1.55- 1.39 (m, 2H), 1.28 (s, 9H), 1.07-0.89 (m, 2H) |
| 87 | 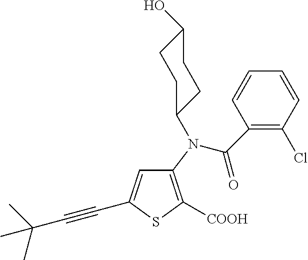 [SMILES: CC(C)(C)C#Cc1cc(N(C(=O)c2ccccc2Cl)C2CCC(O)CC2)c(C(=O)O)s1] | C | | C | A | 461.94 | ¹H NMR (400 MHz, DMSO-d₆): δ 13.66 (s, 1H), 7.36-7.12 (m, 5H), 4.57 (s, 1H), 4.41(s, 1H), 3.30-3.22 (m, 1H), 2.09-1.70 (m, 5H), 1.54-1.39 (m, 2H), 1.35-1.18 (m, 9H), 1.03-0.89 (m, 1H) |
| 88 | 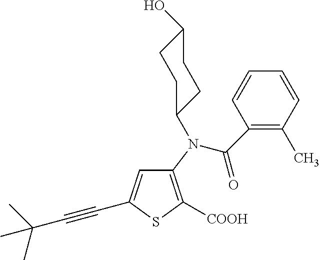 [SMILES: Cc1ccccc1C(=O)N(c1cc(C#CC(C)(C)C)sc1C(=O)O)C1CCC(O)CC1] | B | | C | A | 439.98 | ¹H NMR (400 MHz, DMSO-d₆): δ 13.49 (s, 1H), 7.24 (s, 1H), 7.13-6.92 (m, 4H), 4.56 (d, 1H), 4.48- 4.36 (m, 1H), 3.31- 3.22 (m, 1H), 2.23 (s, 3H), 2.06-1.72 (m, 5H), 1.54-1.39 (m, 2H), 1.33-1.31 (m, 1H), 1.26 (s, 9H), 1.04-0.90 (m, 2H) |
| 89 | 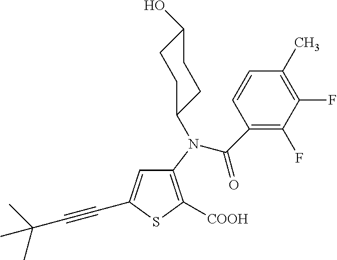 [SMILES: Cc1ccc(C(=O)N(c2cc(C#CC(C)(C)C)sc2C(=O)O)C2CCC(O)CC2)c(F)c1F] | A | | C | B | 475.97 | ¹H NMR (400 MHz, DMSO-d₆): δ 13.56 (s, 1H), 7.25 (s, 1H), 7.03-6.83 (m, 2H), 4.56 (d, 1H), 4.45- 4.30 (m, 1H), 3.30- 3.17 (m, 1H), 2.19 (s, 3H), 2.05-1.65 (m, 5H), 1.58-1.38 (m, 1H), 1.37-1.15 (m, 10H), 1.07- 0.90 (m, 1H) |
| 95 | 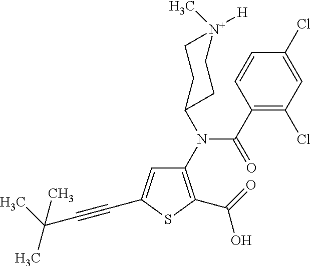 [SMILES: C[NH+]1CCC(N(C(=O)c2ccc(Cl)cc2Cl)c2cc(C#CC(C)(C)C)sc2C(=O)O)CC1] | A | | C | A | 494.73 | ¹H NMR (400 MHz, DMSO-d6): δ [ppm] 7.55 (s, 1H), 7.39- 7.22 (m, 2H), 7.15 (s, 1H), 4.75-4.61 (m, 1H), 3.50- 2.90 (m, 4H), 2.69 (s, 3H), 2.30-1.50 (m, 4H), 1.29 (s, 9H) |
| 96 | 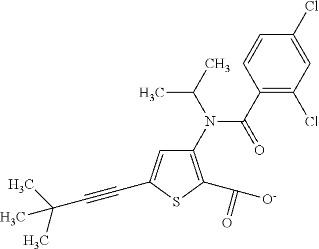 [SMILES: CC(C)N(C(=O)c1ccc(Cl)cc1Cl)c1cc(C#CC(C)(C)C)sc1C(=O)[O-]] | A | | B | B | | |
| 97 | 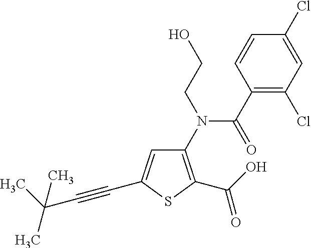 [SMILES: CC(C)(C)C#Cc1cc(N(CCO)C(=O)c2ccc(Cl)cc2Cl)c(C(=O)O)s1] | B | | B | B | 441.82 | |

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

From the foregoing description, one skilled in the art can easily ascertain the essential characteristics of this invention and, without departing from the spirit and scope thereof, can make various changes and modifications of the invention to adapt it to various usages and conditions.

1. A compound of formula (I) or a pharmaceutically acceptable salt thereof: 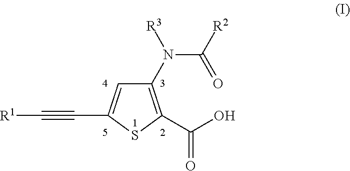
wherein,
R¹ is C₁₋₆ alkyl or C₃₋₆ cycloalkyl;
R² is a phenyl which is unsubstituted or substituted one or more times by R¹⁰;
R³ is C₁₋₆ alkyl which is unsubstituted or substituted one or more times by R¹¹; 6 membered heterocycle which is unsubstituted or substituted one or more times by R¹², C₃₋₆ cycloalkyl which is unsubstituted or substituted one or more times by R¹²; or 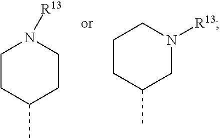
R¹⁰ is halogen, C₁₋₃ alkyl, halogenated C₁₋₃ alkyl, C₁₋₃-alkoxy, —NH₂, hydroxyl, nitro, cyano or CH₃COO—;
R¹¹ is halogen, oxo, C₁₋₆ alkoxy, C₃₋₆ cycloalkyl, 5-6 membered heterocycle, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄, alkyl, —NHCOC₁₋₄ alkyl, carboxy, hydroxyl, nitro, azido, cyano, —S(O)₀₋₂H, —S(O)₀₋₂C₁₋₄ alkyl, —NHSO₂C₁₋₄ alkyl;
R¹² is OH, oxo, halogen, C₁₋₆-alkoxy, C₁₋₆-alkyl, C₁₋₆-alkyl-CO—NH—, C₁₋₆-alkyl-CO—N(C₁₋₆-alkyl)-, 3-6 membered heterocycle, or a 5-10 membered heteroaryl; and
R¹³ is C₁₋₆-alkyl, C₃₋₇-cycloalkyl, halogenated C₁₋₆-alkyl, C₁₋₆-alkyl-CO—, —S(O)₀₋₂C₁₋₄ alkyl, 5-10 membered heteroaryl or C₆₋₁₄-aryl.

2. A compound according to claim 1, wherein R¹ is C₁₋₆ alkyl.

3. (canceled)

4. A compound according to claim 2, wherein R¹ is methyl, ethyl, propyl, isopropyl, butyl, sec-butyl, tert-butyl cyclopropyl, cyclobutyl, cyclopentyl, or cyclohexyl.

5. (canceled)

6. A compound according to claim 4, wherein R¹ is tert-butyl.

7. A compound according to claim 6, wherein R² is phenyl which is substituted in 2 and 4 position.

8. A compound according to claim 6, wherein R² is phenyl which is substituted in 4 position.

9. A compound according to claim 8, wherein R¹⁰ is halogen, C₁₋₃ alkyl or —NH₂.

10. (canceled)

11. (canceled)

12. (canceled)

13. A compound according to claim 1, wherein R² is 2,4-dichlorophenyl, 2-fluoro-4-chlorophenyl, 2,4-dimethylphenyl, 2-hydroxy-4-methylphenyl, 2-methyl-4-chlorophenyl, 2-bromo-4-methylphenyl, 3-fluoro-4-methylphenyl, 2-amino-4-chlorophenyl, 4-chlorophenyl, 4-methylphenyl or 4-trifluoromethylphenyl.

14. (canceled)

15. (canceled)

16. (canceled)

17. (canceled)

18. (canceled)

19. A compound according to claim 1, wherein R³ is cyclohexyl which is unsubstituted or substituted one or more times by R¹².

20. (canceled)

21. (canceled)

22. A compound according to claim 1, wherein R³ is 1,3-dimethoxy isopropyl, 1-methoxy isopropyl, methoxy ethyl, 2,2,2-trifluoroethyl, 2,2-difluoroethyl, 2-fluoroethyl, or tetrahydrofuran, tetrahydropyran or 1,3-dioxane.

23. (canceled)

24. A compound according to claim 19, wherein R³ is cyclohexyl which is substituted one or more times by OH, oxo, halogen, C₁₋₆-alkoxy, C₁₋₆-alkyl, or triazolyl.

25. (canceled)

26. (canceled)

27. (canceled)

28. (canceled)

29. (canceled)

30. (canceled)

31. (canceled)

32. A compound according to claim 19, wherein R³ is cyclohexyl which is substituted in the 4-position by OH, C₁₋₆-alkyl, or C₁₋₆-alkoxy wherein the 4-position substituent is in the trans position relative to the amino group.

33. (canceled)

34. A compound according to claim 1, wherein
R³ is 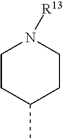
and R¹³ is C₁₋₆-alkyl, C₃₋₇-cycloalkyl, halogenated C₁₋₆-alkyl, C₁₋₆-alkyl-CO—, —S(O)₀₋₂C₁₋₄ alkyl, 5-10 membered heteroaryl or C₆₋₁₄-aryl.

35. A compound according to claim 1, wherein R¹¹ is halogen, oxo, C₁₋₆alkoxy, C₃₋₆ cycloalkyl, 5-6 membered heterocycle, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄, alkyl, —NHCOC₁₋₄ alkyl or hydroxyl.

36. A compound according to claim 35, wherein R¹¹ is halogen, methoxy or hydroxyl.

37. (canceled)

38. (canceled)

39. (canceled)

40. A compound according to claim 1, wherein R¹² is methyl, ethyl, propyl, isopropyl, cyclopropyl, cyclobutyl, butyl, sec-butyl, tert-butyl.

41. A compound according to claim 40, wherein R¹² is methyl, ethyl, isopropyl, OH, methoxy or ethoxy.

42. (canceled)

43. (canceled)

44. (canceled)

45. (canceled)

46. (canceled)

47. A compound according to claim 1 wherein
R¹ is tert-butyl;
R² is 2,4-dichlorophenyl; and,
R³ is cyclohexyl which is substituted one or more times by OH, F, C₁₋₄-alkoxy, or C₁₋₄-alkyl.

48. A compound selected from:
3-[(2,4-Dichloro-benzoyl)-isopropyl-amino]-5-(3,3-dimethyl-but-1-ynyl)-thiophene-2-carboxylic acid
3-[(2,4-Dichloro-benzoyl)-(trans-4-hydroxy-cyclohexyl)-amino]-5-(3,3-dimethyl-but-1-ynyl)-thiophene-2-carboxylic acid
3-[(2,4-Dichloro-benzoyl)-(cis-4-hydroxy-cyclohexyl)-amino]-5-(3,3-dimethyl-but-1-ynyl)-thiophene-2-carboxylic acid
3-[(2,4-Dichloro-benzoyl)-(trans-4-methoxy-cyclohexyl)-amino]-5-(3,3-dimethyl-but-1-ynyl)-thiophene-2-carboxylic acid
3-[(2,4-Dichloro-benzoyl)-(cis-4-methoxy-cyclohexyl)-amino]-5-(3,3-dimethyl-but-1-ynyl)-thiophene-2-carboxylic acid
3-[(2,4-Dichloro-benzoyl)-(trans-4-[1,2,3]triazol-1-yl-cyclohexyl)-amino]-5-(3,3-dimethyl-but-1-ynyl)-thiophene-2-carboxylic acid
3-[(2,4-Dichloro-benzoyl)-(cis-4-[1,2,3]triazol-1-yl-cyclohexyl)-amino]-5-(3,3-dimethyl-but-1-ynyl)-thiophene-2-carboxylic acid
3-[(2,4-Dichloro-benzoyl)-(trans-4-[1,2,4]triazol-1-yl-cyclohexyl)-amino]-5-(3,3-dimethyl-but-1-ynyl)-thiophene-2-carboxylic acid
3-[(2,4-Dichloro-benzoyl)-(cis-4-[1,2,4]triazol-1-yl-cyclohexyl)-amino]-5-(3,3-dimethyl-but-1-ynyl)-thiophene-2-carboxylic acid
3-[(2,4-Dichloro-benzoyl)-(trans-4-fluoro-cyclohexyl)-amino]-5-(3,3-dimethyl-but-1-ynyl)-thiophene-2-carboxylic acid
3-[(2,4-Dichloro-benzoyl)-(4,4-difluoro-cyclohexyl)-amino]-5-(3,3-dimethyl-but-1-ynyl)-thiophene-2-carboxylic acid
3-[(2,4-Dichloro-benzoyl)-(1-methyl-piperidin-4-yl)-amino]-5-(3,3-dimethyl-but-1-ynyl)-thiophene-2-carboxylic acid hydrochloride
3-[(2,4-Dichloro-benzoyl)-(4-oxo-cyclohexyl)-amino]-5-(3,3-dimethyl-but-1-ynyl)-thiophene-2-carboxylic acid
3-[(2,4-Dichloro-benzoyl)-(1,4-dioxa-spiro[4.5]dec-8-yl)-amino]-5-(3,3-dimethyl-but-1-ynyl)-thiophene-2-carboxylic acid
3-[(2,4-Dichloro-benzoyl)-(trans-4-methyl-cyclohexyl)-amino]-5-(3,3-dimethyl-but-1-ynyl)-thiophene-2-carboxylic acid
3-[(4-Chloro-benzoyl)-(trans-4-hydroxy-cyclohexyl)-amino]-5-(3,3-dimethyl-but-1-ynyl)-thiophene-2-carboxylic acid
3-[(4-Chloro-2-fluoro-benzoyl)-(trans-4-hydroxy-cyclohexyl)-amino]-5-(3,3-dimethyl-but-1-ynyl)-thiophene-2-carboxylic acid
3-[(2,4-Dimethyl-benzoyl)-(trans-4-hydroxy-cyclohexyl)-amino]-5-(3,3-dimethyl-but-1-ynyl)-thiophene-2-carboxylic acid
5-(3,3-Dimethyl-but-1-ynyl)-3-[(trans-4-hydroxy-cyclohexyl)-(4-methyl-benzoyl)-amino]-thiophene-2-carboxylic acid
5-(3,3-Dimethyl-but-1-ynyl)-3-[(trans-4-hydroxy-cyclohexyl)-(2-hydroxy-4-methyl-benzoyl)-amino]-thiophene-2-carboxylic acid
3-[(4-Chloro-2-methyl-benzoyl)-(trans-4-hydroxy-cyclohexyl)-amino]-5-(3,3-dimethyl-but-1-ynyl)-thiophene-2-carboxylic acid
3-[(4-Chloro-benzoyl)-(trans-4-hydroxy-cyclohexyl)-amino]-5-(3,3-dimethyl-but-1-ynyl)-thiophene-2-carboxylic acid
3-[(2-Bromo-4-methyl-benzoyl)-(trans-4-hydroxy-cyclohexyl)-amino]-5-(3,3-dimethyl-but-1-ynyl)-thiophene-2-carboxylic acid
5-(3,3-Dimethyl-but-1-ynyl)-3-[(3-fluoro-4-methyl-benzoyl)-(trans-4-hydroxy-cyclohexyl)-amino]-thiophene-2-carboxylic acid
3-[(2,4-Dichloro-benzoyl)-[1,3]dioxan-5-yl-amino]-5-(3,3-dimethyl-but-1-ynyl)-thiophene-2-carboxylic acid
3-[(2,4-Dichloro-benzoyl)-(tetrahydro-pyran-4-yl)-amino]-5-(3,3-dimethyl-but-1-ynyl)-thiophene-2-carboxylic acid
3-[(2,4-Dichloro-benzoyl)-(2-methoxy-1-methoxymethyl-ethyl)-amino]-5-(3,3-dimethyl-but-1-ynyl)-thiophene-2-carboxylic acid
3-[Cyclopropyl-(2,4-dichloro-benzoyl)-amino]-5-(3,3-dimethyl-but-1-ynyl)-thiophene-2-carboxylic acid
3-[(2-Amino-4-chloro-benzoyl)-(trans-4-hydroxy-cyclohexyl)-amino]-5-(3,3-dimethyl-but-1-ynyl)-thiophene-2-carboxylic acid
3-[(2,4-Dichloro-benzoyl)-methyl-amino]-5-(3,3-dimethyl-but-1-ynyl)-thiophene-2-carboxylic acid
3-[Cyclohexyl-(2,4-dichloro-benzoyl)-amino]-5-(3,3-dimethyl-but-1-ynyl)-thiophene-2-carboxylic acid
3-[(2,4-Dichloro-benzoyl)-(1-pyrimidin-2-yl-piperidin-4-yl)-amino]-5-(3,3-dimethyl-but-1-ynyl)-thiophene-2-carboxylic acid
3-[(2-Amino-4-chloro-benzoyl)-(trans-4-methoxymethyl-cyclohexyl)-amino]-5-(3,3-dimethyl-but-1-ynyl)-thiophene-2-carboxylic acid
3-[(2,4-Dichloro-benzoyl)-(1-methanesulfonyl-piperidin-4-yl)-amino]-5-(3,3-dimethyl-but-1-ynyl)-thiophene-2-carboxylic acid
3-[(2-Amino-4-chloro-benzoyl)-(trans-4-isobutyrylamino-cyclohexyl)-amino]-5-(3,3-dimethyl-but-1-ynyl)-thiophene-2-carboxylic acid
3-[(2,4-Dichloro-benzoyl)-(1-isobutyryl-piperidin-4-yl)-amino]-5-(3,3-dimethyl-but-1-ynyl)-thiophene-2-carboxylic acid
3-[(2,4-Dichloro-benzoyl)-(2-methyl-3-oxo-2-aza-spiro[4.5]dec-8-yl)-amino]-5-(3,3-dimethyl-but-1-ynyl)-thiophene-2-carboxylic acid
3-[(2,4-Dichloro-benzoyl)-(3-oxo-2-aza-spiro[4.5]dec-8-yl)-amino]-5-(3,3-dimethyl-but-1-ynyl)-thiophene-2-carboxylic acid
3-[(2,4-Dichloro-benzoyl)-ethyl-amino]-5-(3,3-dimethyl-but-1-ynyl)-thiophene-2-carboxylic acid
3-[Cyclopropylmethyl-(2,4-dichloro-benzoyl)-amino]-5-(3,3-dimethyl-but-1-ynyl)-thiophene-2-carboxylic acid
3-[Cyclobutyl-(2,4-dichloro-benzoyl)-amino]-5-(3,3-dimethyl-but-1-ynyl)-thiophene-2-carboxylic acid
3-[(2,4-Dichloro-benzoyl)-(tetrahydro-furan-3-yl)-amino]-5-(3,3-dimethyl-but-1-ynyl)-thiophene-2-carboxylic acid
3-[(2,4-Dichloro-benzoyl)-(2-methoxy-1-methyl-ethyl)-amino]-5-(3,3-dimethyl-but-1-ynyl)-thiophene-2-carboxylic acid
3-[(2,4-Dichloro-benzoyl)-(2-methoxy-ethyl)-amino]-5-(3,3-dimethyl-but-1-ynyl)-thiophene-2-carboxylic acid
3-[Cyclopentyl-(2,4-dichloro-benzoyl)-amino]-5-(3,3-dimethyl-but-1-ynyl)-thiophene-2-carboxylic acid
3-[(2,4-Dichloro-benzoyl)-(2,2,2-trifluoro-ethyl)-amino]-5-(3,3-dimethyl-but-1-ynyl)-thiophene-2-carboxylic acid
3-{(2,4-Dichloro-benzoyl)-[1-(2,2-difluoro-ethyl)-piperidin-4-yl]-amino}-5-(3,3-dimethyl-but-1-ynyl)-thiophene-2-carboxylic acid
3-[tert-Butyl-(2,4-dichloro-benzoyl)-amino]-5-(3,3-dimethyl-but-1-ynyl)-thiophene-2-carboxylic acid
3-{(2,4-Dichloro-benzoyl)-[1-(2-fluoro-ethyl)-piperidin-4-yl]-amino}-5-(3,3-dimethyl-but-1-ynyl)-thiophene-2-carboxylic acid
3-{(2-Amino-4-chloro-benzoyl)-[4-(isobutyryl-methyl-amino)-cyclohexyl]-amino}-5-(3,3-dimethyl-but-1-ynyl)-thiophene-2-carboxylic acid
5-(3,3-Dimethyl-but-1-ynyl)-3-[(4-fluoro-benzoyl)-(4-hydroxy-cyclohexyl)-amino]-thiophene-2-carboxylic acid
3-[(4-Chloro-3-fluoro-benzoyl)-(4-hydroxy-cyclohexyl)-amino]-5-(3,3-dimethyl-but-1-ynyl)-thiophene-2-carboxylic acid
3-[(2-Chloro-4-methyl-benzoyl)-(4-hydroxy-cyclohexyl)-amino]-5-(3,3-dimethyl-but-1-ynyl)-thiophene-2-carboxylic acid
5-(3,3-Dimethyl-but-1-ynyl)-3-[(2-fluoro-4-methyl-benzoyl)-(4-hydroxy-cyclohexyl)-amino]-thiophene-2-carboxylic acid
and
pharmaceutically acceptable salts thereof.

49. A compound according to claim 1, represented by any one of the following structural formulae or a pharmaceutically acceptable salt thereof:

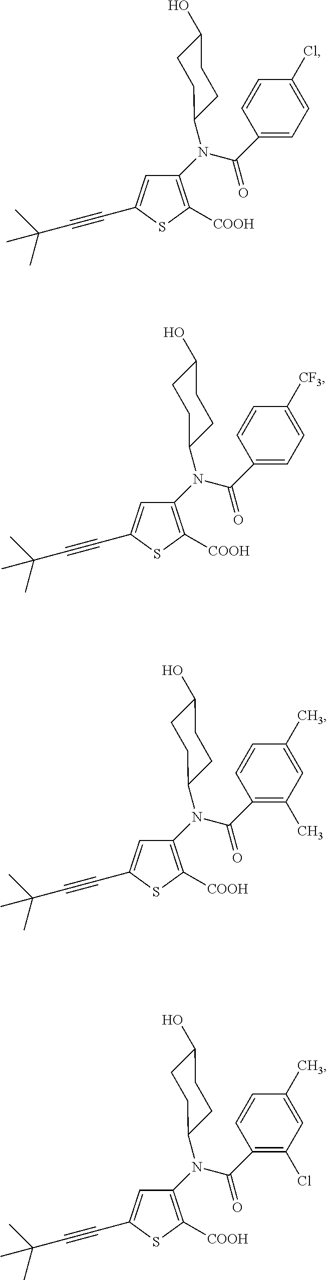

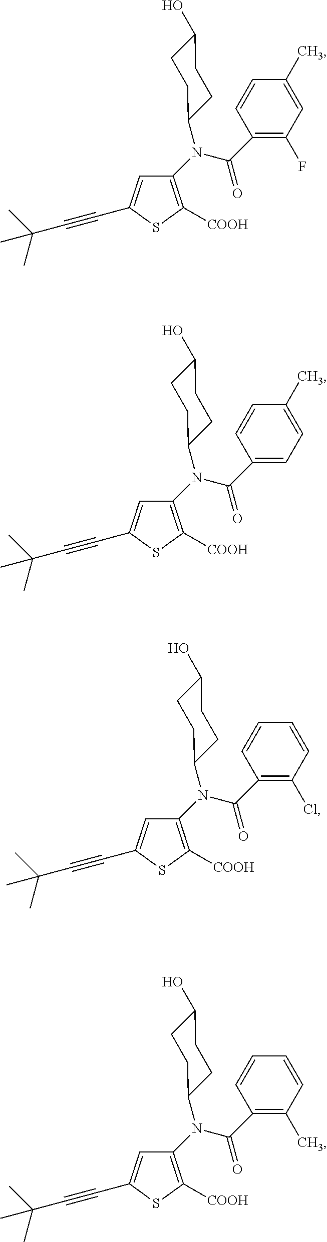 [SMILES: Cc1ccc(C(=O)N(c2cc(C#CC(C)(C)C)sc2C(=O)O)C2CCC(O)CC2)c(F)c1 ; Cc1ccc(C(=O)N(c2cc(C#CC(C)(C)C)sc2C(=O)O)C2CCC(O)CC2)cc1 ; CC(C)(C)C#Cc1cc(N(C(=O)c2ccccc2Cl)C2CCC(O)CC2)c(C(=O)O)s1 ; Cc1ccccc1C(=O)N(c1cc(C#CC(C)(C)C)sc1C(=O)O)C1CCC(O)CC1]

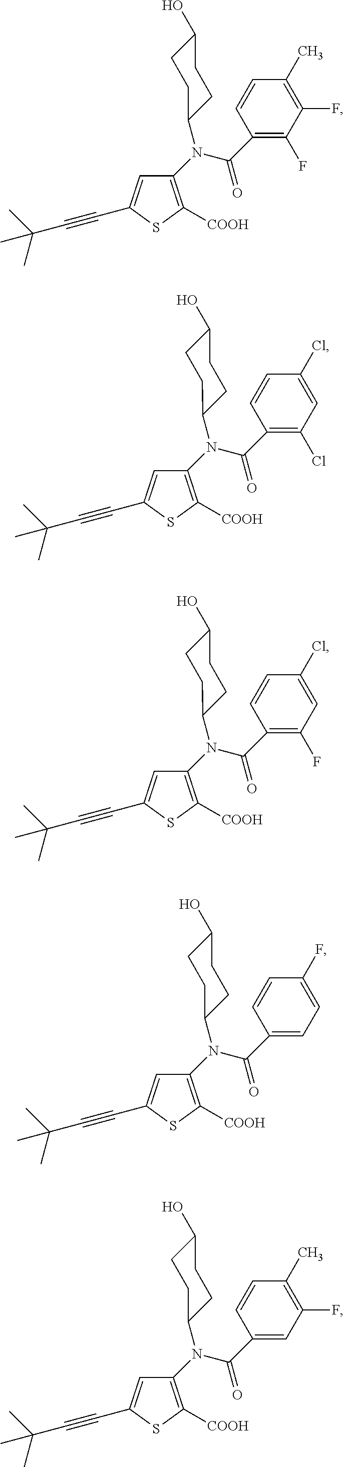 [SMILES: Cc1ccc(C(=O)N(c2cc(C#CC(C)(C)C)sc2C(=O)O)C2CCC(O)CC2)c(F)c1F ; CC(C)(C)C#Cc1cc(N(C(=O)c2ccc(Cl)cc2Cl)C2CCC(O)CC2)c(C(=O)O)s1 ; CC(C)(C)C#Cc1cc(N(C(=O)c2ccc(Cl)cc2F)C2CCC(O)CC2)c(C(=O)O)s1 ; CC(C)(C)C#Cc1cc(N(C(=O)c2ccc(F)cc2)C2CCC(O)CC2)c(C(=O)O)s1 ; Cc1ccc(C(=O)N(c2cc(C#CC(C)(C)C)sc2C(=O)O)C2CCC(O)CC2)cc1F]

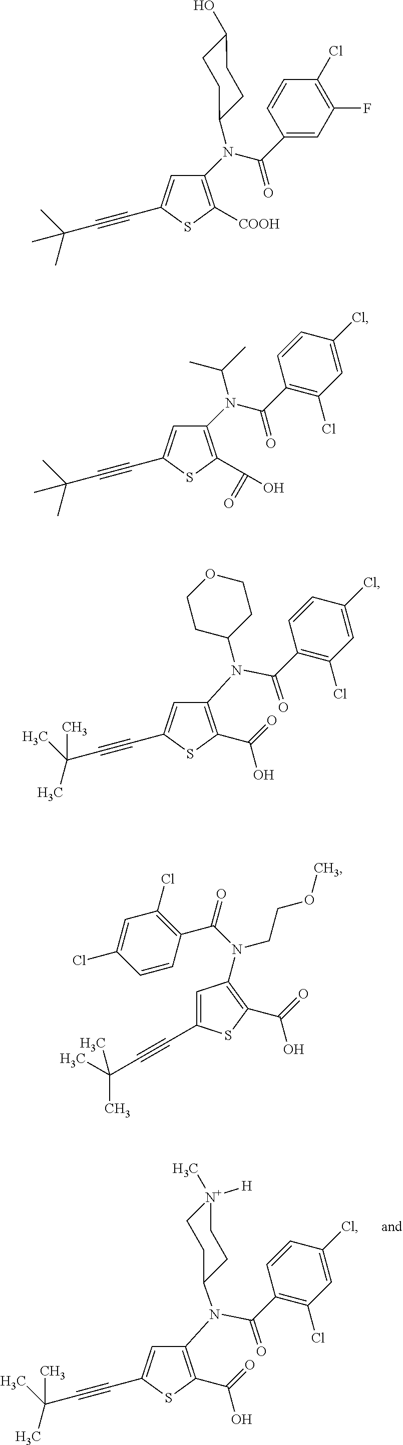 [SMILES: CC(C)(C)C#Cc1cc(N(C(=O)c2ccc(Cl)c(F)c2)C2CCC(O)CC2)c(C(=O)O)s1 ; CC(C)N(C(=O)c1ccc(Cl)cc1Cl)c1cc(C#CC(C)(C)C)sc1C(=O)O ; CC(C)(C)C#Cc1cc(N(C(=O)c2ccc(Cl)cc2Cl)C2CCOCC2)c(C(=O)O)s1 ; COCCN(C(=O)c1ccc(Cl)cc1Cl)c1cc(C#CC(C)(C)C)sc1C(=O)O ; C[NH+]1CCC(N(C(=O)c2ccc(Cl)cc2Cl)c2cc(C#CC(C)(C)C)sc2C(=O)O)CC1]

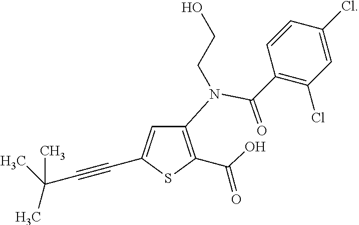 [SMILES: CC(C)(C)C#Cc1cc(N(CCO)C(=O)c2ccc(Cl)cc2Cl)c(C(=O)O)s1]

50. (canceled)

51. (canceled)

52. A pharmaceutical composition comprising at least one compound according to any one of claim 1 and at least one pharmaceutically acceptable carrier or excipient.

53. A pharmaceutical combination comprising at least one compound according to any one of claim 1 and at least one additional agent.

54. A pharmaceutical combination according to claim 53 wherein said at least one additional agent is chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents and inhibitors of internal ribosome entry site (IRES).

55. A pharmaceutical combination according to claim 53 wherein said at least one additional agent is chosen from interferon α 1A, interferon α 1B, interferon α 2A (Roferon), PEG-interferon α 2A (Pegasys), interferon α 2B (Intron A) or PEG-interferon α 2B (Peg-Intron).

56. A pharmaceutical combination according to claim 53 wherein said at least one additional agent is chosen from standard or pegylated interferon α (Roferon, Pegasys, Intron A, Peg-Intron) in combination with ribavirin.

57. (canceled)

58. (canceled)

59. A method for treating or preventing a Flaviviridae viral infection in a host comprising administering a therapeutically effective amount of a compound according to claim 1.

60. A method for inhibiting or reducing the activity of viral polymerase in a host comprising administering a therapeutically effective amount of a compound according to claim 1.

61. A method for treating or preventing a HCV infection in a host comprising administering a therapeutically effective amount of a compound according to claim 1.

62. (canceled)

63. (canceled)

64. (canceled)

65. (canceled)

66. (canceled)

67. (canceled)

68. (canceled)

69. (canceled)
